(19) United States
(12) Patent Application Publication
(10) Pub. No.: US 2024/0287526 A1
(43) Pub. Date: Aug. 29, 2024

(54) COMPOSITIONS AND METHODS FOR DELIVERING THERAPEUTIC OLIGONUCLEOTIDES TO THE CENTRAL NERVOUS SYSTEM

(71) Applicant: Atalanta Therapeutics, Inc., Boston, MA (US)

(72) Inventors: Matthew HASSLER, Boston, MA (US); Garth A. KINBERGER, Hyde Park, MA (US); Bruno Miguel Da Cruz GODINHO, Reading, MA (US)

(21) Appl. No.: 18/566,983

(22) PCT Filed: Jun. 2, 2022
(86) PCT No.: PCT/US2022/032014

§ 371 Date: Dec. 4, 2023

Related U.S. Application Data
(60) Provisional application No. 63/195,993, filed on Jun. 2, 2021.

(51) Int. Cl. C12N 15/113

(52) U.S. Cl. CPC C12N 15/1138; C12N 15/113; C12N 2310/11; C12N 2310/14; C12N 2310/141; C12N 2310/315; C12N 2310/321; C12N 2310/322; C12N 2310/351; C12N 2310/52; C12N 2310/531

(57) ABSTRACT

The present disclosure provides single- or double-stranded therapeutic oligonucleotides (e.g., siRNAs, shRNAs, miRNAs, gRNAs, and ASOs) having a plurality of cationic binding sites that are partially or fully saturated by a plurality of divalent cations (e.g., Ba²⁺, Be²⁺, Ca²⁺, Cu²⁺, Mg²⁺, Mn²⁺, Ni²⁺, or Zn²⁺, or a combination thereof). The therapeutic oligonucleotides may contain specific patterns of nucleoside modifications and internucleoside linkage modifications, as pharmaceutical compositions including the same. The siRNA molecules may be branched siRNA molecules, such as di-branched, tri-branched, or tetra-branched siRNA molecules. The disclosed siRNA molecules may further feature a 5′ phosphorus stabilizing moiety and/or a hydrophobic moiety. Additionally, the disclosure provides methods for delivering the siRNA molecule of the disclosure to the central nervous system of a subject, such as a subject identified as having a disease.

### TECHNICAL FIELD

This disclosure relates to interfering RNA molecules (e.g., short interfering RNA molecules) that are ionically bound to one or more divalent cations, as well as methods of delivering the same to the central nervous system of a subject in need of modulation of one or more genes.

### BACKGROUND

In many species, introduction of double-stranded RNA induces potent and specific gene silencing by way of RNA interference (RNAi). This phenomenon occurs in both plants and animals and has roles in viral defense and transposon silencing mechanisms. For example, short interfering RNAs (siRNAs), which are generally much shorter than the target gene, have been shown to be effective at gene silencing and are, therefore, useful as therapeutic agents for silencing genes to restore genetic and biochemical pathway activity from a disease state towards a normal, healthy state. However, delivery of interfering RNA molecules, such as siRNA, to a subject, particularly to the subject's central nervous system, carries the risk of toxic side effects, including seizures, tremors, and hyperactive motor behaviors, among others. There remains a need for interfering RNA molecules that effectuate reduced toxicity upon administration to a subject in need thereof.

### SUMMARY OF THE DISCLOSURE

The present disclosure provides interfering RNA molecules containing a plurality of cationic binding sites that are partially or fully saturated with divalent cations (e.g., divalent metal cations). For example, interfering RNA molecules of the disclosure may contain one or more phosphodiester internucleoside linkages and/or an analog thereof, such as a phosphorothioate internucleoside linkage, in which oxyanion moieties are electrostatically neutralized by ionic bonding to a divalent metal cation, such as Ba²⁺, Be²⁺, Ca²⁺, Cu²⁺, Mg²⁺, Mn²⁺, Ni²⁺, or Zn²⁺. It has presently been discovered that incorporating one or more divalent cations into an interfering RNA molecule (e.g., a short interfering RNA (siRNA)) prior to administration of the interfering RNA molecule to a subject (e.g., a mammalian subject, such as a human) strongly suppresses toxic side effects that may otherwise be caused by administration of an interfering RNA molecule, particularly in the subject's central nervous system.

Using the compositions and methods of the disclosure, therapeutic oligonucleotides, such as interfering RNA molecules (e.g., siRNAs), may be formulated by incorporating one or more divalent cations by way of ionic bonding. This may be achieved by titrating an aqueous solution or suspension containing a therapeutic oligonucleotide (e.g., an interfering RNA molecule such as an siRNA) with an aqueous solution or suspension containing a divalent cation of interest. The ensuing therapeutic oligonucleotide, having cationic binding sites that are partially or fully saturated with the divalent cation, may then be administered to a subject, e.g., in the form of an aqueous solution or suspension, so as to modulate expression of a desired gene. Advantageously, the subject may experience side effects of a reduced frequency and/or magnitude than would be observed in a subject that is administered the same therapeutic oligonucleotide but lacking the one or more divalent cations.

In a first aspect, the present disclosure provides a method for delivering therapeutic oligonucleotides (e.g., siRNAs or antisense oligonucleotides, among others described herein) to a subject (e.g., a mammalian subject, such as a human) by administering the therapeutic oligonucleotide to the subject in the form of a salt containing one or more divalent cations. The therapeutic oligonucleotide may include a plurality of cationic binding sites that are partially or fully saturated by the one or more divalent cations.

In some embodiments, the therapeutic oligonucleotide is administered to the subject in the form of an aqueous solution or in the form of a suspension. The therapeutic oligonucleotide may be administered to the subject systemically or directly to the subject's central nervous system (CNS). For example, the therapeutic oligonucleotide may be administered to the subject's cerebral spinal fluid (CSF), spinal cord, brain parenchyma, cortex, cerebellum, basal ganglia, caudate, putamen, thalamus, globus pallidus, substantia nigra, or another brain structure. In some embodiments, the therapeutic oligonucleotide is administered intrathecally, intracerebroventricularly, intrastriatally, or by intra-cisterna magna injection via catheterization. In some embodiments, the therapeutic oligonucleotide is administered intrathecally. In some embodiments, the therapeutic oligonucleotide is administered intracerebroventricularly.

In a second aspect, the disclosure provides a therapeutic oligonucleotide (e.g., an siRNA) formulated as a salt containing one or more divalent cations. The therapeutic oligonucleotide may contain a plurality of cationic binding sites that are partially or fully saturated by the one or more divalent cations.

In some embodiments of either of the foregoing aspects of the disclosure, the degree of saturation of the cationic binding sites by the one or more divalent cations is from about 10% to about 100% (e.g., from about 20% to about 100%, from about 30% to about 100%, from about 40% to about 100%, from about 50% to about 100%, from about 60% to about 100%, from about 70% to about 100%, from about 80% to about 100%, or from about 90% to about 100%).

In some embodiments, the cationic binding site is located within an internucleoside linkage, such as a phosphodiester linkage and/or a phosphorothioate linkage. For example, the cationic binding site may be an oxyanion moiety within a phosphodiester linage or phosphorothioate linkage.

In some embodiments, the one or more divalent cations are characterized as having an ionic radius ranging from about 30 picometers to about 150 picometers (e.g., from about 30 picometers to about 140 picometers, from about 40 picometers to about 130 picometers, from about 50 picometers to about 120 picometers, from about 60 picometers to about 110 picometers, from about 60 picometers to about 100 picometers, or from about 60 picometers to about 90 picometers).

In some embodiments, the one or more divalent cations include a hard Lewis acid. In some embodiments, the one or more divalent cations includes Ba²⁺, Be²⁺, Ca²⁺, Cu²⁺, Mg²⁺, Mn²⁺, Ni²⁺, or Zn²⁺, or a combination thereof.

In some embodiments, the one or more divalent cations includes Ba²⁺. In some embodiments, the one or more divalent cations includes Be²⁺. In some embodiments, the one or more divalent cations includes Ca²⁺. In some embodiments, the one or more divalent cations includes Cu²⁺. In some embodiments, the one or more divalent cations includes Mg²⁺. In some embodiments, the one or more divalent cations includes Mn²⁺. In some embodiments, the one or more divalent cations includes Ni²⁺. In some embodiments, the one or more divalent cations includes Zn²⁺.

In some embodiments, the one or more divalent cations includes Ca²⁺ and Mg²⁺, optionally wherein the ratio of Ca²⁺ to Mg²⁺ is from 1:100 to 100:1 (e.g., 1:75, 1:50, 1:25, 1:10, 1:5, 1:1, 5:1, 10:1, 25:1. 50:1, 75:1, or 100:1). In some embodiments, the Ca²⁺ and Mg²⁺ are present in a 1:1 ratio.

In some embodiments, the one or more divalent cations displace water from a cationic binding site of the therapeutic oligonucleotide.

In some embodiments, the therapeutic oligonucleotide is an siRNA, a short hairpin RNA (shRNA), a microRNA (miRNA), a CRISPR guide RNA, or an RNA antisense oligonucleotide (ASO). In some embodiments, the therapeutic oligonucleotide is an interfering RNA molecule. In some embodiments, the interfering RNA molecule is an siRNA. The siRNA may be a branched siRNA, such as a di-branched siRNA molecule, a tri-branched siRNA molecule, or a tetra-branched siRNA molecule.

In some embodiments, the di-branched siRNA molecule is represented by any one of Formulas I-III:

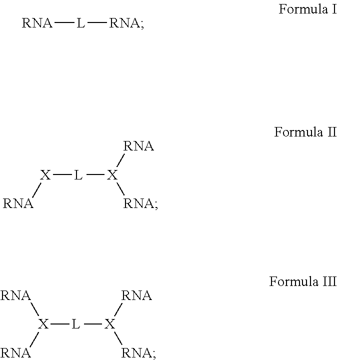

wherein each RNA is, independently, an siRNA molecule, L is a linker, and each X, independently, represents a branch point moiety.

In some embodiments, the di-branched siRNA molecule is represented by Formula I. In some embodiments, the di-branched siRNA molecule is represented by Formula II. In some embodiments, the di-branched siRNA molecule is represented by Formula III.

In some embodiments, the tri-branched siRNA molecule is represented by any one of Formulas IV-VII:

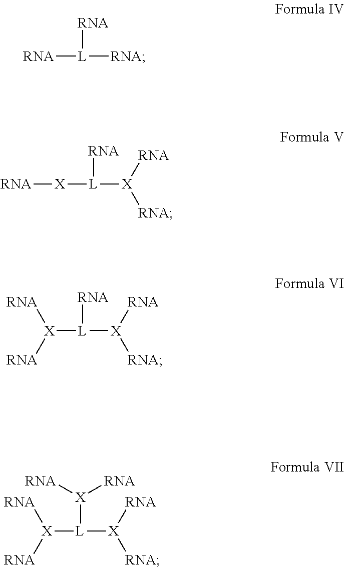

wherein each RNA is, independently, an siRNA molecule, L is a linker, and each X, independently, represents a branch point moiety.

In some embodiments, the tri-branched siRNA molecule is represented by Formula IV. In some embodiments, the tri-branched siRNA molecule is represented by Formula V. In some embodiments, the tri-branched siRNA molecule is represented by Formula VI. In some embodiments, the tri-branched siRNA molecule is represented by Formula VII.

In some embodiments, the tetra-branched siRNA molecule is represented by any one of Formulas VIII-XII:

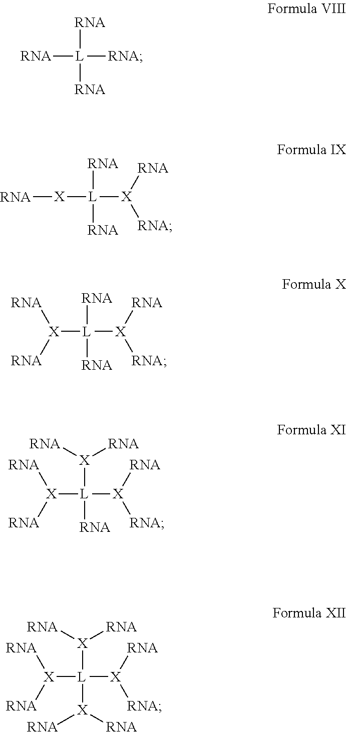

wherein each RNA is, independently, an siRNA molecule, L is a linker, and each X, independently, represents a branch point moiety.

In some embodiments, the tetra-branched siRNA molecule is represented by Formula VIII. In some embodiments, the tetra-branched siRNA molecule is represented by Formula IX. In some embodiments, the tetra-branched siRNA molecule is represented by Formula X. In some embodiments, the tetra-branched siRNA molecule is represented by Formula XI. In some embodiments, the tetra-branched siRNA molecule is represented by Formula XII.

In some embodiments, the linker is selected from a group consisting of one or more contiguous subunits of an ethylene glycol (e.g., polyethylene glycol (PEG), such as, e.g., triethylene glycol (TrEG) or tetraethylene glycol (TEG)), alkyl, carbohydrate, block copolymer, peptide, RNA, and DNA.

In some embodiments, the linker is an ethylene glycol oligomer. In some embodiments, the linker is an alkyl oligomer. In some embodiments, the linker is a carbohydrate oligomer. In some embodiments, the linker is a block copolymer. In some embodiments, the linker is a peptide oligomer. In some embodiments, the linker is an RNA oligomer. In some embodiments, the linker is a DNA oligomer.

In some embodiments, the ethylene glycol oligomer is a PEG. In some embodiments, the PEG is a TrEG. In some embodiments, the PEG is a TEG.

In some embodiments, the oligomer or copolymer contains 2 to 20 contiguous subunits (e.g., 2, 3, 4, 5, 6, 7, 8, 9, 10, 11, 12, 13, 14, 15, 16, 17, 18, 19, or 20 contiguous subunits).

In some embodiments, the linker attaches one or more (e.g., 1, 2, 3, 4, or more) siRNA molecules by way of a covalent bond-forming moiety.

In some embodiments, the covalent bond-forming moiety is selected from the group consisting of an alkyl, ester, amide, carbamate, phosphonate, phosphate, phosphorothioate, phosphoroamidate, triazole, urea, and formacetal.

In some embodiments, the linker includes a structure of Formula L1:

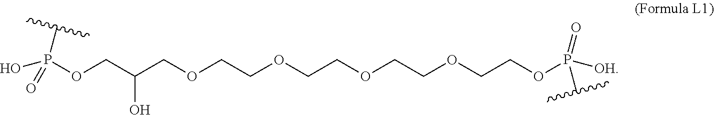 [SMILES: *P(=O)(O)OCCOCCOCCOCCOCC(O)COP(*)(=O)O]

In some embodiments, the linker includes a structure of Formula L2:

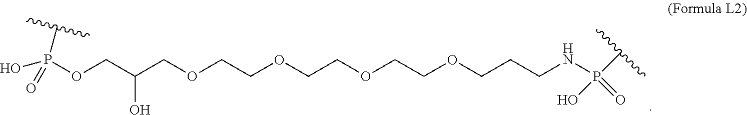 [SMILES: *P(=O)(O)NCCCOCCOCCOCCOCC(O)COP(*)(=O)O]

In some embodiments, the linker includes a structure of Formula L3:

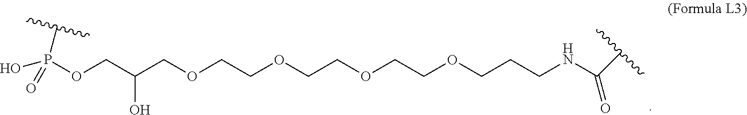 [SMILES: *C(=O)NCCCOCCOCCOCCOCC(O)COP(*)(=O)O]

In some embodiments, the linker includes a structure of Formula L4:

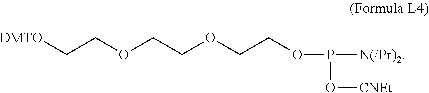

In some embodiments, the linker includes a structure of Formula L5:

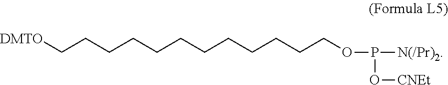

In some embodiments, the linker includes a structure of Formula L6:

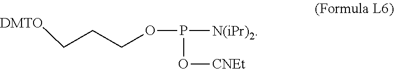

In some embodiments, the linker includes a structure of Formula L7:

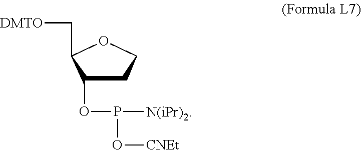

In some embodiments, the linker includes a structure of Formula L8:

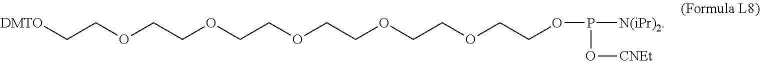

In some embodiments, the linker includes a structure of Formula L9:

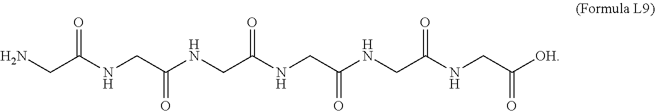 [SMILES: NCC(=O)NCC(=O)NCC(=O)NCC(=O)NCC(=O)NCC(=O)O]

In some embodiments, the therapeutic oligonucleotide includes an antisense strand and a sense strand having complementarity to the antisense strand, or the therapeutic oligonucleotide is an ASO including an antisense strand alone. In some embodiments, the antisense strand and sense strand further include alternating 2′-O-methyl and 2′-fluoro ribonucleosides.

In some embodiments, the interfering RNA antisense strand includes a region represented by the following chemical Formula, in the 5′-to-3′ direction:

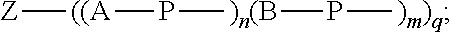

wherein Z is a 5′ phosphorus stabilizing moiety; each A is, independently, a 2′-O-methyl (2′-O-Me) ribonucleoside; each B is, independently, a 2′-fluoro-ribonucleoside; each P is, independently, an internucleoside linkage selected from a phosphodiester linkage and a phosphorothioate linkage; n is an integer from 1 to 5 (e.g., 1, 2, 3, 4, or 5); m is an integer from 1 to 5 (e.g., 1, 2, 3, 4, or 5); and q is an integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).

In some embodiments, the interfering RNA antisense strand includes a structure represented by Formula I, wherein Formula I is, in the 5′-to-3′ direction:

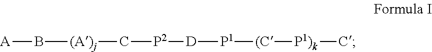

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

In some embodiments of Formula I, the antisense strand includes a structure represented by Formula AI, wherein Formula A1 is, in the 5′-to-3′ direction:

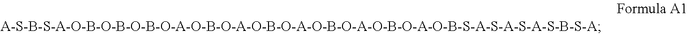

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the interfering RNA antisense strand includes, includes a structure represented by Formula II, wherein Formula II is, in the 5′-to-3′ direction:

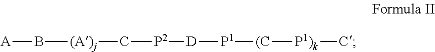

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

In some embodiments of Formula II, the antisense strand includes a structure represented by Formula A2, wherein Formula A2 is, in the 5′-to-3′ direction:

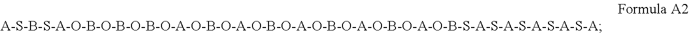

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the therapeutic oligonucleotide sense strand includes a structure represented by Formula III, wherein Formula III is, in the 5′-to-3′ direction:

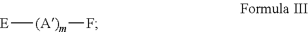

wherein E is represented by the formula (C—P¹)₂;
F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D;
A′, C, D, P¹, and P² are as defined in Formula II; and
m is an integer from 1 to 7.

In some embodiments of Formula III, the sense strand includes a structure represented by Formula S1, wherein Formula S1 is, in the 5′-to-3′ direction:

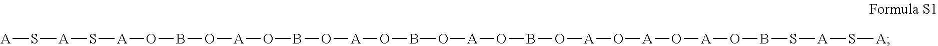

wherein A represents a 2′-O-Me ribonucleoside, B represents a 2′-F ribonucleoside, O represents a phosphodiester internucleoside linkage, and S represents a phosphorothioate internucleoside linkage.

In some embodiments of Formula III, the sense strand includes a structure represented by Formula S2, wherein Formula S2 is, in the 5′-to-3′ direction:

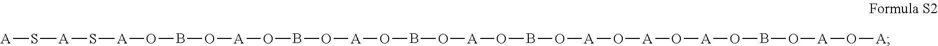

wherein A represents a 2′-O-Me ribonucleoside, B represents a 2′-F ribonucleoside, O represents a phosphodiester internucleoside linkage, and S represents a phosphorothioate internucleoside linkage.

In some embodiments of Formula III, the sense strand includes a structure represented by Formula S3, wherein Formula S3 is, in the 5′-to-3′ direction:

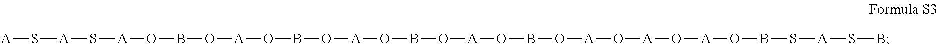

wherein A represents a 2′-O-Me ribonucleoside, B represents a 2′-F ribonucleoside, O represents a phosphodiester internucleoside linkage, and S represents a phosphorothioate internucleoside linkage.

In some embodiments of Formula III, the sense strand includes a structure represented by Formula S4, wherein Formula S4 is, in the 5′-to-3′ direction:

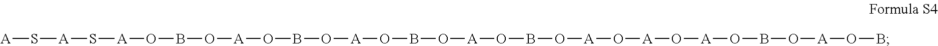

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the therapeutic oligonucleotide antisense strand includes a structure represented by Formula IV, wherein Formula IV is, in the 5′-to-3′ direction:

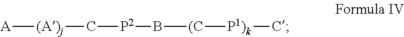

wherein A is represented by the formula C—P¹-D-P¹;
each A′ is represented by the formula C—P²-D-P²;
B is represented by the formula D-P¹—C—P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

In some embodiments of Formula IV, the antisense strand includes a structure represented by Formula A3, wherein Formula A3 is, in the 5′-to-3′ direction:

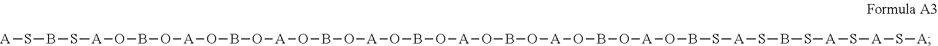

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the therapeutic oligonucleotide sense strand includes a structure represented by Formula V, wherein Formula V is, in the 5′-to-3′ direction:

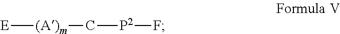

wherein E is represented by the formula (C—P¹)₂;
F is represented by the formula D-P¹—C—P¹—C, D-P²—C—P²—C, D-P¹—C—P¹-D, or D-P²—C—P²-D;
A′, C, D, P¹ and P² are as defined in Formula IV; and
m is an integer from 1 to 7.

In some embodiments of Formula V, the sense strand includes a structure represented by Formula S5, wherein Formula S5 is, in the 5′-to-3′ direction:

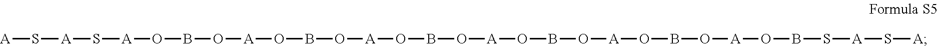

wherein A represents a 2′-O-Me ribonucleoside, B represents a 2′-F ribonucleoside, O represents a phosphodiester internucleoside linkage, and S represents a phosphorothioate internucleoside linkage.

In some embodiments of Formula V, the sense strand includes a structure represented by Formula S6, wherein Formula S6 is, in the 5′-to-3′ direction:

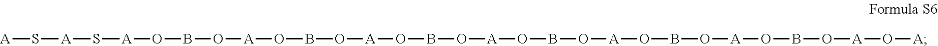

wherein A represents a 2′-O-Me ribonucleoside, B represents a 2′-F ribonucleoside, O represents a phosphodiester internucleoside linkage, and S represents a phosphorothioate internucleoside linkage.

In some embodiments of Formula V, the sense strand includes a structure represented by Formula S7, wherein Formula S7 is, in the 5′-to-3′ direction:

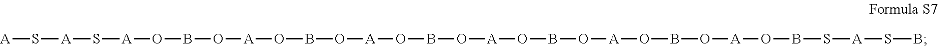

wherein A represents a 2′-O-Me ribonucleoside, B represents a 2′-F ribonucleoside, O represents a phosphodiester internucleoside linkage, and S represents a phosphorothioate internucleoside linkage.

In some embodiments of Formula V, the sense strand includes a structure represented by Formula S8, wherein Formula S8 is, in the 5′-to-3′ direction:

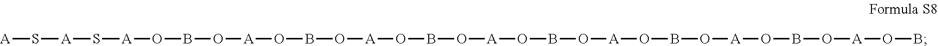

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the therapeutic oligonucleotide antisense strand includes a structure represented by Formula VI, wherein Formula VI is, in the 5′-to-3′ direction:

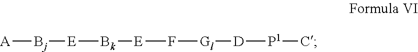

wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7;
k is an integer from 1 to 7; and
l is an integer from 1 to 7.

In some embodiments of Formula VI, the antisense strand includes a structure represented by Formula A4, wherein Formula A4 is, in the 5′-to-3′ direction:

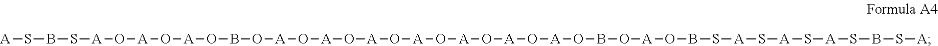

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the therapeutic oligonucleotide sense strand includes a structure represented by Formula VII, wherein Formula VII is, in the 5′-to-3′ direction:

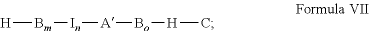

wherein A′ is represented by the formula C—P²-D-P²;
each H is represented by the formula (C—P¹)₂;
each I is represented by the formula (D-P²);
B, C, D, P¹ and P² are as defined in Formula VI;
m is an integer from 1 to 7;
n is an integer from 1 to 7; and
is an integer from 1 to 7.

In some embodiments of Formula VII, the sense strand includes a structure represented by Formula s9, wherein Formula S9 is, in the 5′-to-3′ direction:

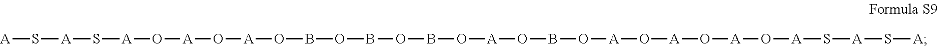

wherein A represents a 2′-O-Me ribonucleoside, B represents a 2′-F ribonucleoside, 0 represents a phosphodiester internucleoside linkage, and S represents a phosphorothioate internucleoside linkage.

In some embodiments, the antisense strand further comprises a 5′-phosphorus stabilizing moiety at the 5′ end of the antisense strand. In some embodiments, the sense strand further comprises a 5′-phosphorus stabilizing moiety at the 5′ end of the sense strand.

In some embodiments, the 5′phosphorus stabilizing moiety is represented in any one of Formula IX-XVI:

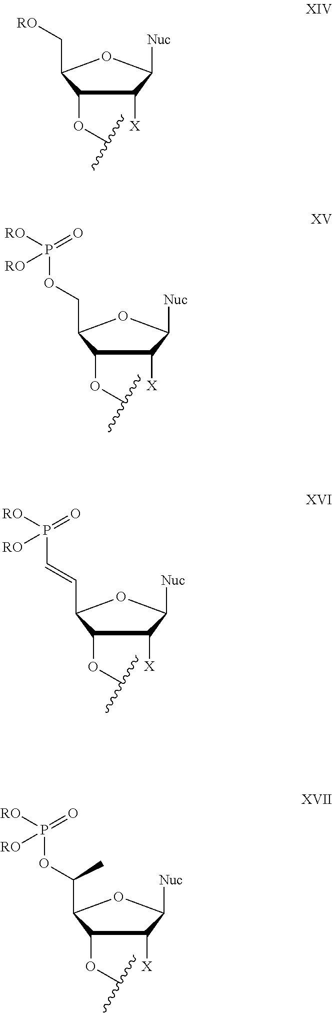

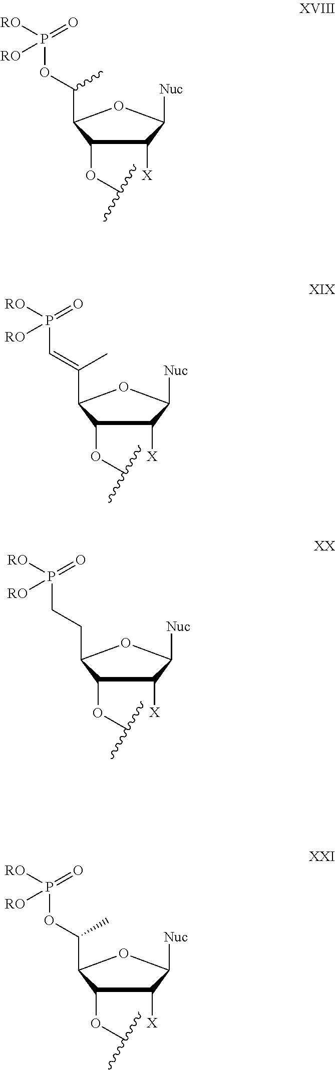

wherein Nuc represents a nucleobase, such as adenine, uracil, guanine, thymine, or cytosine, and R represents optionally substituted alkyl, optionally substituted alkenyl, or optionally substituted alkynyl (e.g., optionally substituted C1-C6 alkyl, optionally substituted C2-C6 alkenyl, or optionally substituted C2-C6 alkynyl), phenyl, benzyl, hydroxy, or hydrogen.

In some embodiments, the 5′phosphorus stabilizing moiety is (E)-vinylphosphonate as represented in Formula XVI.

In some embodiments, n is from 1 to 4. In some embodiments, n is from 1 to 3. In some embodiments, n is from 1 to 2. In some embodiments, n is 1.

In some embodiments, m is from 1 to 4. In some embodiments, m is from 1 to 3. In some embodiments, m is from 1 to 2. In some embodiments, m is 1.

In some embodiments, n and m are each 1.

In some embodiments, 50% or more of the ribonucleotides in the antisense strand are 2′-O-Me ribonucleotides (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 60% or more of the ribonucleotides in the antisense strand are 2′-O-Me ribonucleotides (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 70% or more of the ribonucleotides in the antisense strand are 2′-O-Me ribonucleotides (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 80% or more of the ribonucleotides in the antisense strand are 2′-O-Me ribonucleotides (e.g., 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 90% or more of the ribonucleotides in the antisense strand are 2′-O-Me ribonucleotides (e.g., 90%, 91%, 92%, 93%, 94%, 95%, 96%, 97%, 98%, 99%, or 100% of the ribonucleotides in the antisense strand may be 2′-O-Me ribonucleotides).

In some embodiments, 10% or less of the internucleoside linkages are phosphodiester linkages or phosphorothioate linkages. In some embodiments, at least 10%, 20%, 30%, 40%, 50%, 60%, 70%, 80%, or 90% of the internucleoside linkages are phosphodiester linkages or phosphorothioate linkages.

In some embodiments, 100% of the internucleoside linkages are phosphodiester linkages or phosphorothioate linkages.

In some embodiments, 9 internucleoside linkages are phosphodiester linkages or phosphorothioate linkages.

In some embodiments, the length of the antisense strand is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the length of the antisense strand is 20 nucleotides. In some embodiments, the length of the antisense strand is 21 nucleotides. In some embodiments, the length of the antisense strand is 22 nucleotides. In some embodiments, the length of the antisense strand is 23 nucleotides. In some embodiments, the length of the antisense strand is 24 nucleotides. In some embodiments, the length of the antisense strand is 25 nucleotides. In some embodiments, the length of the antisense strand is 26 nucleotides. In some embodiments, the length of the antisense strand is 27 nucleotides. In some embodiments, the length of the antisense strand is 28 nucleotides. In some embodiments, the length of the antisense strand is 29 nucleotides. In some embodiments, the length of the antisense strand is 30 nucleotides.

In some embodiments, the siRNA molecules of the branched compound are joined to one another by way of a linker (e.g., an ethylene glycol oligomer, such as tetraethylene glycol). In some embodiments, the siRNA molecules of the branched compound are joined to one another by way of a linker between the sense strand of one siRNA molecule and the sense strand of the other siRNA molecule. In some embodiments, the siRNA molecules are joined by way of linkers between the antisense strand of one siRNA molecule and the antisense strand of the other siRNA molecule. In some embodiments, the siRNA molecules of the branched compound are joined to one another by way of a linker between the sense strand of one siRNA molecule and the antisense strand of the other siRNA molecule.

In some embodiments, the length of the sense strand is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 18 nucleotides (e.g., 14 nucleotides, 15 nucleotides, 16 nucleotides, 17 nucleotides, or 18 nucleotides). In some embodiments, the length of the sense strand is 15 nucleotides. In some embodiments, the length of the sense strand is 16 nucleotides. In some embodiments, the length of the sense strand is 17 nucleotides. In some embodiments, the length of the sense strand is 18 nucleotides. In some embodiments, the length of the sense strand is 19 nucleotides. In some embodiments, the length of the sense strand is 20 nucleotides. In some embodiments, the length of the sense strand is 21 nucleotides. In some embodiments, the length of the sense strand is 22 nucleotides. In some embodiments, the length of the sense strand is 23 nucleotides. In some embodiments, the length of the sense strand is 24 nucleotides. In some embodiments, the length of the sense strand is 25 nucleotides. In some embodiments, the length of the sense strand is 26 nucleotides. In some embodiments, the length of the sense strand is 27 nucleotides. In some embodiments, the length of the sense strand is 28 nucleotides. In some embodiments, the length of the sense strand is 29 nucleotides. In some embodiments, the length of the sense strand is 30 nucleotides.

In some embodiments, four internucleoside linkages are phosphorothioate linkages.

In some embodiments, the antisense strand is 18 nucleotides in length and the sense strand is 14 nucleotides in length. In some embodiments, the antisense strand is 18 nucleotides in length and the sense strand is 15 nucleotides in length. In some embodiments, the antisense strand is 18 nucleotides in length and the sense strand is 16 nucleotides in length. In some embodiments, the antisense strand is 18 nucleotides in length and the sense strand is 17 nucleotides in length. In some embodiments, the antisense strand is 18 nucleotides in length and the sense strand is 18 nucleotides in length. In some embodiments, the antisense strand is 19 nucleotides in length and the sense strand is 14 nucleotides in length. In some embodiments, the antisense strand is 19 nucleotides in length and the sense strand is 15 nucleotides in length. In some embodiments, the antisense strand is 19 nucleotides in length and the sense strand is 16 nucleotides in length. In some embodiments, the antisense strand is 19 nucleotides in length and the sense strand is 17 nucleotides in length. In some embodiments, the antisense strand is 19 nucleotides in length and the sense strand is 18 nucleotides in length. In some embodiments, the antisense strand is 19 nucleotides in length and the sense strand is 19 nucleotides in length. In some embodiments, the antisense strand is 20 nucleotides in length and the sense strand is 14 nucleotides in length. In some embodiments, the antisense strand is 20 nucleotides in length and the sense strand is 15 nucleotides in length. In some embodiments, the antisense strand is 20 nucleotides in length and the sense strand is 16 nucleotides in length. In some embodiments, the antisense strand is 20 nucleotides in length and the sense strand is 17 nucleotides in length. In some embodiments, the antisense strand is 20 nucleotides in length and the sense strand is 18 nucleotides in length. In some embodiments, the antisense strand is 20 nucleotides in length and the sense strand is 19 nucleotides in length. In some embodiments, the antisense strand is 20 nucleotides in length and the sense strand is 20 nucleotides in length. In some embodiments, the antisense strand is 21 nucleotides in length and the sense strand is 14 nucleotides in length. In some embodiments, the antisense strand is 21 nucleotides in length and the sense strand is 15 nucleotides in length. In some embodiments, the antisense strand is 21 nucleotides in length and the sense strand is 16 nucleotides in length. In some embodiments, the antisense strand is 21 nucleotides in length and the sense strand is 17 nucleotides in length. In some embodiments, the antisense strand is 21 nucleotides in length and the sense strand is 18 nucleotides in length. In some embodiments, the antisense strand is 21 nucleotides in length and the sense strand is 19 nucleotides in length. In some embodiments, the antisense strand is 21 nucleotides in length and the sense strand is 20 nucleotides in length. In some embodiments, the antisense strand is 21 nucleotides in length and the sense strand is 21 nucleotides in length. In some embodiments, the antisense strand is 22 nucleotides in length and the sense strand is 14 nucleotides in length. In some embodiments, the antisense strand is 22 nucleotides in length and the sense strand is 15 nucleotides in length. In some embodiments, the antisense strand is 22 nucleotides in length and the sense strand is 16 nucleotides in length. In some embodiments, the antisense strand is 22 nucleotides in length and the sense strand is 17 nucleotides in length. In some embodiments, the antisense strand is 22 nucleotides in length and the sense strand is 18 nucleotides in length. In some embodiments, the antisense strand is 22 nucleotides in length and the sense strand is 19 nucleotides in length. In some embodiments, the antisense strand is 22 nucleotides in length and the sense strand is 20 nucleotides in length. In some embodiments, the antisense strand is 22 nucleotides in length and the sense strand is 21 nucleotides in length. In some embodiments, the antisense strand is 22 nucleotides in length and the sense strand is 22 nucleotides in length. In some embodiments, the antisense strand is 23 nucleotides in length and the sense strand is 14 nucleotides in length. In some embodiments, the antisense strand is 23 nucleotides in length and the sense strand is 15 nucleotides in length. In some embodiments, the antisense strand is 23 nucleotides in length and the sense strand is 16 nucleotides in length. In some embodiments, the antisense strand is 23 nucleotides in length and the sense strand is 17 nucleotides in length. In some embodiments, the antisense strand is 23 nucleotides in length and the sense strand is 18 nucleotides in length. In some embodiments, the antisense strand is 23 nucleotides in length and the sense strand is 19 nucleotides in length. In some embodiments, the antisense strand is 23 nucleotides in length and the sense strand is 20 nucleotides in length. In some embodiments, the antisense strand is 23 nucleotides in length and the sense strand is 21 nucleotides in length. In some embodiments, the antisense strand is 23 nucleotides in length and the sense strand is 22 nucleotides in length. In some embodiments, the antisense strand is 23 nucleotides in length and the sense strand is 23 nucleotides in length. In some embodiments, the antisense strand is 24 nucleotides in length and the sense strand is 14 nucleotides in length. In some embodiments, the antisense strand is 24 nucleotides in length and the sense strand is 15 nucleotides in length. In some embodiments, the antisense strand is 24 nucleotides in length and the sense strand is 16 nucleotides in length. In some embodiments, the antisense strand is 24 nucleotides in length and the sense strand is 17 nucleotides in length. In some embodiments, the antisense strand is 24 nucleotides in length and the sense strand is 18 nucleotides in length. In some embodiments, the antisense strand is 24 nucleotides in length and the sense strand is 19 nucleotides in length. In some embodiments, the antisense strand is 24 nucleotides in length and the sense strand is 20 nucleotides in length. In some embodiments, the antisense strand is 24 nucleotides in length and the sense strand is 21 nucleotides in length. In some embodiments, the antisense strand is 24 nucleotides in length and the sense strand is 22 nucleotides in length. In some embodiments, the antisense strand is 24 nucleotides in length and the sense strand is 23 nucleotides in length. In some embodiments, the antisense strand is 24 nucleotides in length and the sense strand is 24 nucleotides in length. In some embodiments, the antisense strand is 25 nucleotides in length and the sense strand is 14 nucleotides in length. In some embodiments, the antisense strand is 25 nucleotides in length and the sense strand is 15 nucleotides in length. In some embodiments, the antisense strand is 25 nucleotides in length and the sense strand is 16 nucleotides in length. In some embodiments, the antisense strand is 25 nucleotides in length and the sense strand is 17 nucleotides in length. In some embodiments, the antisense strand is 25 nucleotides in length and the sense strand is 18 nucleotides in length. In some embodiments, the antisense strand is 25 nucleotides in length and the sense strand is 19 nucleotides in length. In some embodiments, the antisense strand is 25 nucleotides in length and the sense strand is 20 nucleotides in length. In some embodiments, the antisense strand is 25 nucleotides in length and the sense strand is 21 nucleotides in length. In some embodiments, the antisense strand is 25 nucleotides in length and the sense strand is 22 nucleotides in length. In some embodiments, the antisense strand is 25 nucleotides in length and the sense strand is 23 nucleotides in length. In some embodiments, the antisense strand is 25 nucleotides in length and the sense strand is 24 nucleotides in length. In some embodiments, the antisense strand is 25 nucleotides in length and the sense strand is 25 nucleotides in length. In some embodiments, the antisense strand is 26 nucleotides in length and the sense strand is 14 nucleotides in length. In some embodiments, the antisense strand is 26 nucleotides in length and the sense strand is 15 nucleotides in length. In some embodiments, the antisense strand is 26 nucleotides in length and the sense strand is 16 nucleotides in length. In some embodiments, the antisense strand is 26 nucleotides in length and the sense strand is 17 nucleotides in length. In some embodiments, the antisense strand is 26 nucleotides in length and the sense strand is 18 nucleotides in length. In some embodiments, the antisense strand is 26 nucleotides in length and the sense strand is 19 nucleotides in length. In some embodiments, the antisense strand is 26 nucleotides in length and the sense strand is 20 nucleotides in length. In some embodiments, the antisense strand is 26 nucleotides in length and the sense strand is 21 nucleotides in length. In some embodiments, the antisense strand is 26 nucleotides in length and the sense strand is 22 nucleotides in length. In some embodiments, the antisense strand is 26 nucleotides in length and the sense strand is 23 nucleotides in length. In some embodiments, the antisense strand is 26 nucleotides in length and the sense strand is 24 nucleotides in length. In some embodiments, the antisense strand is 26 nucleotides in length and the sense strand is 25 nucleotides in length. In some embodiments, the antisense strand is 26 nucleotides in length and the sense strand is 26 nucleotides in length. In some embodiments, the antisense strand is 27 nucleotides in length and the sense strand is 14 nucleotides in length. In some embodiments, the antisense strand is 27 nucleotides in length and the sense strand is 15 nucleotides in length. In some embodiments, the antisense strand is 27 nucleotides in length and the sense strand is 16 nucleotides in length. In some embodiments, the antisense strand is 27 nucleotides in length and the sense strand is 17 nucleotides in length. In some embodiments, the antisense strand is 27 nucleotides in length and the sense strand is 18 nucleotides in length. In some embodiments, the antisense strand is 27 nucleotides in length and the sense strand is 19 nucleotides in length. In some embodiments, the antisense strand is 27 nucleotides in length and the sense strand is 20 nucleotides in length. In some embodiments, the antisense strand is 27 nucleotides in length and the sense strand is 21 nucleotides in length. In some embodiments, the antisense strand is 27 nucleotides in length and the sense strand is 22 nucleotides in length. In some embodiments, the antisense strand is 27 nucleotides in length and the sense strand is 23 nucleotides in length. In some embodiments, the antisense strand is 27 nucleotides in length and the sense strand is 24 nucleotides in length. In some embodiments, the antisense strand is 27 nucleotides in length and the sense strand is 25 nucleotides in length. In some embodiments, the antisense strand is 27 nucleotides in length and the sense strand is 26 nucleotides in length. In some embodiments, the antisense strand is 27 nucleotides in length and the sense strand is 27 nucleotides in length. In some embodiments, the antisense strand is 28 nucleotides in length and the sense strand is 14 nucleotides in length. In some embodiments, the antisense strand is 28 nucleotides in length and the sense strand is 15 nucleotides in length. In some embodiments, the antisense strand is 28 nucleotides in length and the sense strand is 16 nucleotides in length. In some embodiments, the antisense strand is 28 nucleotides in length and the sense strand is 17 nucleotides in length. In some embodiments, the antisense strand is 28 nucleotides in length and the sense strand is 18 nucleotides in length. In some embodiments, the antisense strand is 28 nucleotides in length and the sense strand is 19 nucleotides in length. In some embodiments, the antisense strand is 28 nucleotides in length and the sense strand is 20 nucleotides in length. In some embodiments, the antisense strand is 28 nucleotides in length and the sense strand is 21 nucleotides in length. In some embodiments, the antisense strand is 28 nucleotides in length and the sense strand is 22 nucleotides in length. In some embodiments, the antisense strand is 28 nucleotides in length and the sense strand is 23 nucleotides in length. In some embodiments, the antisense strand is 28 nucleotides in length and the sense strand is 24 nucleotides in length. In some embodiments, the antisense strand is 28 nucleotides in length and the sense strand is 25 nucleotides in length. In some embodiments, the antisense strand is 28 nucleotides in length and the sense strand is 26 nucleotides in length. In some embodiments, the antisense strand is 28 nucleotides in length and the sense strand is 27 nucleotides in length. In some embodiments, the antisense strand is 28 nucleotides in length and the sense strand is 28 nucleotides in length. In some embodiments, the antisense strand is 29 nucleotides in length and the sense strand is 14 nucleotides in length. In some embodiments, the antisense strand is 29 nucleotides in length and the sense strand is 15 nucleotides in length. In some embodiments, the antisense strand is 29 nucleotides in length and the sense strand is 16 nucleotides in length. In some embodiments, the antisense strand is 29 nucleotides in length and the sense strand is 17 nucleotides in length. In some embodiments, the antisense strand is 29 nucleotides in length and the sense strand is 18 nucleotides in length. In some embodiments, the antisense strand is 29 nucleotides in length and the sense strand is 19 nucleotides in length. In some embodiments, the antisense strand is 29 nucleotides in length and the sense strand is 20 nucleotides in length. In some embodiments, the antisense strand is 29 nucleotides in length and the sense strand is 21 nucleotides in length. In some embodiments, the antisense strand is 29 nucleotides in length and the sense strand is 22 nucleotides in length. In some embodiments, the antisense strand is 29 nucleotides in length and the sense strand is 23 nucleotides in length. In some embodiments, the antisense strand is 29 nucleotides in length and the sense strand is 24 nucleotides in length. In some embodiments, the antisense strand is 29 nucleotides in length and the sense strand is 25 nucleotides in length. In some embodiments, the antisense strand is 29 nucleotides in length and the sense strand is 26 nucleotides in length. In some embodiments, the antisense strand is 29 nucleotides in length and the sense strand is 27 nucleotides in length. In some embodiments, the antisense strand is 29 nucleotides in length and the sense strand is 28 nucleotides in length. In some embodiments, the antisense strand is 29 nucleotides in length and the sense strand is 29 nucleotides in length. In some embodiments, the antisense strand is 30 nucleotides in length and the sense strand is 14 nucleotides in length. In some embodiments, the antisense strand is 30 nucleotides in length and the sense strand is 15 nucleotides in length. In some embodiments, the antisense strand is 30 nucleotides in length and the sense strand is 16 nucleotides in length. In some embodiments, the antisense strand is 30 nucleotides in length and the sense strand is 17 nucleotides in length. In some embodiments, the antisense strand is 30 nucleotides in length and the sense strand is 18 nucleotides in length. In some embodiments, the antisense strand is 30 nucleotides in length and the sense strand is 19 nucleotides in length. In some embodiments, the antisense strand is 30 nucleotides in length and the sense strand is 20 nucleotides in length. In some embodiments, the antisense strand is 30 nucleotides in length and the sense strand is 21 nucleotides in length. In some embodiments, the antisense strand is 30 nucleotides in length and the sense strand is 22 nucleotides in length. In some embodiments, the antisense strand is 30 nucleotides in length and the sense strand is 23 nucleotides in length. In some embodiments, the antisense strand is 30 nucleotides in length and the sense strand is 24 nucleotides in length. In some embodiments, the antisense strand is 30 nucleotides in length and the sense strand is 25 nucleotides in length. In some embodiments, the antisense strand is 30 nucleotides in length and the sense strand is 26 nucleotides in length. In some embodiments, the antisense strand is 30 nucleotides in length and the sense strand is 27 nucleotides in length. In some embodiments, the antisense strand is 30 nucleotides in length and the sense strand is 28 nucleotides in length. In some embodiments, the antisense strand is 30 nucleotides in length and the sense strand is 29 nucleotides in length. In some embodiments, the antisense strand is 30 nucleotides in length and the sense strand is 30 nucleotides in length.

In some embodiments, administering the therapeutic oligonucleotide to the subject results in silencing of a gene or splice isoform of a gene in the subject. Silencing of a gene may happen by silencing a positive regulator of a gene for which increased expression and/or activity, relative to the level of expression and/or activity observed in a reference subject, is associated with a disease state. Silencing of a gene may happen by silencing of a negative regulator of a gene for which decreased expression and/or activity, relative to the level of expression and/or activity observed in a reference subject, is associated with a disease state. Silencing of a gene may happen by silencing a specific gene or a splice isoform of a specific gene for which overexpression of the gene or splice isoform of the gene, relative to the expression of the gene or splice isoform of the gene in a reference subject, is associated with a disease state.

In some embodiments, the gene or splice isoform of the gene is transcriptionally expressed in the central nervous system of the subject.

In some embodiments, the silencing of the gene or splice isoform of the gene is used to treat a subject diagnosed with a disease of the central nervous system.

In some embodiments, the silencing of the gene or splice isoform of the gene is used to treat a subject diagnosed with a neurodegenerative disease, a neuropsychiatric disease, or other neurological disorder.

In some embodiments, the disease is Huntington's disease. In some embodiments, the disease is Parkinson's disease. In some embodiments, the disease is Alzheimer's disease. In some embodiments, the disease is amyotrophic lateral sclerosis (ALS). In some embodiments, the disease is dementia with Lewy bodies (DLB). In some embodiments, the disease is pure autonomic failure. In some embodiments, the disease is Lewy body dysphagia. In some embodiments, the disease is incidental Lewy body disease (ILBD). In some embodiments, the disease is inherited Lewy body disease. In some embodiments, the disease is olivopontocerebellar atrophy (OPCA). In some embodiments, the disease is striatonigral degeneration. In some embodiments, the disease is Shy-Drager syndrome. In some embodiments, the disease is epilepsy or an epilepsy disorder. In some embodiments, the disease is a prion disease. In some embodiments, the disease is pain or a pain disorder.

In some embodiments, the antisense strand has complementarity sufficient to hybridize a portion of a gene selected from the group consisting of ABCA7, ABI3, ADAM10, APOC1, APOE, AXL, BIN1, C1QA, C3, C90RF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, HTT, IFIT1, IFIT3, IFITM3, IFNAR1, IFNAR2, IGF1, I10RA, IL1A, IL1B, IL1 RAP, INPP5D, ITGAM, ITGAX, KCNT1, LILRB4, LPL, MAPT, MEF2C, MMP12, MS4A4A, MS4A6A, MSH3, NLRP3, NME8, NOS2, PICALM, PILRA, PLCG2, PRNP, PTK2B, SCIMP, SCN9A, SLC24A4, SNCA, SORL1, SPI1, SPP1, SPPL2A, TBK1, TNF, TREM2, TREML2, TYROBP, and ZCWPW1. In some embodiments, the gene is selected from the group consisting of HTT, MAPT, SNCA, C90RF72, APOE, SCN9A, KCNT1, PRNP, and MSH3. In some embodiments, the gene is HTT. In some embodiments, the gene is MAPT. In some embodiments, the gene is SNCA. In some embodiments, the gene is C90RF72. In some embodiments, the gene is APOE. In some embodiments, the gene is SCN9A. In some embodiments, the gene is KCNT1. In some embodiments, the gene is PRNP. In some embodiments, the gene is MSH3.

In some embodiments the subject is a human.

In some embodiments, the therapeutic oligonucleotide comprises one or more atoms having a negative charge and the divalent cation comprises two positive charges. In some embodiments, the ratio of negative charge to positive charge is from 0.75 to 7.5 (e.g., 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5). In some embodiments, the ratio of negative charge to positive charge is from 1.0 to 2.0 (e.g., from 1.0 to 1.9, from 1.0 to 1.8, from 1.0 to 1.7, from 1.0 to 1.6, from 1.0 to 1.5, from 1.0 to 1.4, from 1.0 to 1.3, from 1.0 to 1.2, from 1.0 to 1.1, from 1.1 to 2.0, from 1.2 to 2.0, from 1.3 to 2.0, from 1.4 to 2.0, from 1.5 to 2.0, from 1.6 to 2.0, from 1.7 to 2.0, from 1.8 to 2.0, or from 1.9 to 2.0). In some embodiments, the ratio of negative charge to positive charge is from 0.75 to 6.5 (e.g., from 0.75 to 5.5, from 0.75 to 4.5, from 0.75 to 3.5, from 0.75 to 2.5, from 0.75 to 1.5, or from 0.75 to 1). In some embodiments, the ratio of negative charge to positive charge is from 1 to 7.5 (e.g., from 1.5 to 7.5, from 2.5 to 7.5, from 3.5 to 7.5, from 4.5 to 7.5, from 5.5 to 7.5, or from 6.5 to 7.5). In some embodiments, the molar ratio of therapeutic oligonucleotide to divalent cation is from 1:10 to 1:100 (e.g., from 1:10 to 1:50, from 1:18 to 1:38, from 1:20 to 1:25, 1:25, or 1:20). In some embodiments, the concentration of the one or more divalent cations is from 10 mM to 150 mM (e.g., from 20 mM to 150 mM, from 20 mM to 100 mM, from 25 mM to 150 mM, from 25 mM to 100 mM, from 30 mM to 90 mM, from 35 mM to 85 mM, from 35 mM to 75 mM, from 40 mM to 70 mM, from 40 mM to 65 mM, from 40 mM to 60 mM, or from 40 mM to 50 mM).

In a third aspect, the disclosure provides a method of synthesizing an siRNA molecule formulated as a salt including one or more divalent cations, the method including heating an antisense strand and a sense strand in the presence of one or more divalent cations. In some embodiments, the heating includes heating to at least 90° C. In some embodiments, the siRNA molecule is the siRNA molecule of any of the preceding aspects or embodiments of the disclosure.

In a fourth aspect, the disclosure provides a method of synthesizing an siRNA molecule formulated as a salt including one or more divalent cations, the method including incubating a hybridized siRNA duplex in the presence of one or more divalent cations without heat. In some embodiments, the siRNA molecule is the siRNA molecule of any of the preceding aspects or embodiments of the disclosure.

In a fifth aspect, the disclosure provides an siRNA molecule synthesized by the method of the third or fourth aspect.

In another aspect, the disclosure provides a pharmaceutical composition containing the therapeutic oligonucleotide of the previous aspect of the disclosure in combination with a pharmaceutically acceptable excipient, carrier, or diluent.

In a further aspect, the disclosure provides a kit containing the therapeutic oligonucleotide of the second aspect of the disclosure or the pharmaceutical composition of the preceding aspect. The kit may also include a package insert that instructs a user of the kit to perform a method described herein, such as the method of the aforementioned first aspect of this the disclosure or any embodiments thereof.

### BRIEF DESCRIPTION OF THE DRAWINGS

FIG. 1A is an experimental outline of unilateral ICV injections into FVB/NJ,F mice followed by methods, described herein, for monitoring and scoring of toxicity of the experimental procedure. ICV injections were conducted with either 5 μl or 10 μl of volumes and administered at a flow rate of 0.5 μl/minute with either 10 nmol or 20 nmol of siRNA.

FIG. 1B is a scatter plot of the EvADINT-A score on mice following the experimental ICV procedure. Experimental conditions for each ICV injection include the delivery of duplex siRNA hybridized in the presence in one of the following four ionic conditions: A) Mg2⁺; B) Ca2⁺; C) Mg2⁺ and Ca2⁺; or D) PBS only (control). The higher the score, the more toxic the experimental condition is considered.

FIG. 2A is a bar graph showing mRNA knockdown by a di-siRNA molecule of the disclosure in the presence of various divalent cations. Each condition tested (PBS only, di-siRNA with Mg²⁺, di-siRNA with Ca²⁺, di-siRNA with Mg²⁺ and Ca²⁺, and di-siRNA with PBS) was evaluated for knockdown in four different parts of the brain. The four bars for each condition represent, from left to right, the frontal cortex, the motor cortex, the striatum, and the hippocampus.

FIG. 2B is a bar graph showing dose-dependent knockdown of a gene of interest by a di-siRNA molecule of the disclosure. On the x axis, “control” refers to an untreated control, “PBS” refers to di-siRNA with PBS, “Mg” refers to di-siRNA with Mg²⁺, and “Ca” refers to di-siRNA with Ca²⁺.

FIG. 2C is a scatter plot showing tissue distribution of a di-siRNA molecule of the disclosure in the presence of various divalent cations. Each condition tested (PBS only, di-siRNA with Mg²⁺, di-siRNA with Ca²⁺, di-siRNA with Mg²⁺ and Ca²⁺, and di-siRNA with PBS) was evaluated for uptake in four different parts of the brain. The y axis represents fmols of oligonucleotide per mg of protein as measured in a PNA hybridization assay. Each condition tested (PBS only, di-siRNA with Mg²⁺, di-siRNA with Ca²⁺, di-siRNA with Mg²⁺ and Ca²⁺, and di-siRNA with PBS) was evaluated for uptake in four different brain regions. For each condition tested, the points from left to right indicate the frontal cortex, the motor cortex, the striatum, and the hippocampus, respectively.

FIG. 3A is a scatter plot of the EvADINT-A score in mice treated with 20 nmol of an siRNA molecule in the presence of varying concentrations of Mg²⁺, or with control conditions of Mg²⁺ in PBS. The higher the score, the more toxic the experimental condition was considered. In FIG. 3A, “Added” indicates that the Mg²⁺ was added to the siRNA after hybridization of the siRNA duplex, and “Re-hyb” indicates that Mg²⁺ was added prior to heating the siRNA molecule to rehybridize it. For each concentration of Mg, the osmolality, pH, and ratio of siRNA to Mg is indicated below.

FIG. 3B is a scatter plot of the EvADINT-A score in mice treated with 20 nmol of an siRNA molecule in the presence of varying concentrations of Ca²⁺. The higher the score, the more toxic the experimental condition was considered.

FIG. 3C is a scatter plot of the EvADINT-A score in mice treated with 20 nmol of an siRNA molecule in the presence of varying concentrations of Mg²⁺/Ca²⁺. The higher the score, the more toxic the experimental condition was considered.

FIG. 3D is a scatter plot of the EvADINT-A score in mice treated with varying concentrations of an siRNA molecule in the presence of a constant ratio of Mg²⁺, or with control conditions of siRNA in PBS with no cation. The higher the score, the more toxic the experimental condition was considered. For each experimental condition, the osmolality and pH are indicated below.

### DEFINITIONS

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “nucleic acids” refers to RNA or DNA molecules consisting of a chain of ribonucleotides or deoxyribonucleotides, respectively.

As used herein, the term “carrier nucleic acid” refers to a nucleic acid molecule (e.g., ribonucleic acid) that has sequence complementarity with, and hybridizes with, a therapeutic nucleic acid. As used herein, the term “3′ end” refers to the end of the nucleic acid that contains a hydroxyl group or modified hydroxyl group at the 3′ carbon of the ribose ring.

As used herein, the term “nucleoside” refers to a molecule made up of a heterocyclic base and its sugar.

As used herein, the term “nucleotide” refers to a nucleoside having a phosphate group on its 3′ or 5′ sugar hydroxyl group. Examples of phosphate group variants include, but are not limited to, saturated alkyl phosphonates, unsaturated alkenyl phosphonates, phosphorothioates, and phosphoramidites.

As used herein, the term “siRNA” refers to small interfering RNA duplexes that induce the RNA interference (RNAi) pathway. siRNA molecules may vary in length (generally, between 10 and 30 base pairs) and may contain varying degrees of complementarity to their target mRNA. The term “siRNA” includes duplexes of two separate strands, as well as single strands that optionally form hairpin structures including a duplex region.

As used herein, the term “antisense strand” refers to the strand of the siRNA duplex that contains some degree of complementarity to the target gene.

As used herein, the term “sense strand” refers to the strand of the siRNA duplex that contains complementarity to the antisense strand.

As used herein, the term “divalent cation” refers to a positively charged ion (i.e., a cation) with a valence of 2+. Examples of divalent cations include, Ba²⁺, Be²⁺, Ca²⁺, Cu²⁺, Mg²⁺, Mn²⁺, Ni²⁺, or Zn²⁺ Because of their positive charge, divalent cations typically form ionic bonds with negatively charged atoms (e.g., oxyanion from a phosphate group or phosphorothioate group carrying a unit or partial negative charge).

As used herein, the terms “ionic radius” and “ionic radii” refer to the radius of one or more monoatomic ions (e.g., divalent cations) when measured in the form of its ionic crystal structure. The ionic radius is typically measured in units of picometers or angstroms.

As used herein, the term “salt” refers to any compound containing an ionic association between an anionic component (e.g., oxyanion from a phosphate group or phosphorothioate group carrying a unit or partial negative charge) and a cationic component (e.g., a divalent cation). Salts may have various physical forms. For example, a salt may be a solid, crystalline, ionic compound, or may be in the form of a solution in which the salt is dissolved in a solvent with which the salt's constituent ions are miscible (e.g., water or another polar, protic solvent). Salts may also exist in suspension, such as a suspension formed by contacting (i) a homogenous solution containing the salt of interest and a first solvent with (ii) a second solvent that is not fully miscible with the first solvent. Examples of suspensions are those formed by contacting an aqueous solution containing a salt of interest with a solvent not fully miscible with water, such as an organic solvent containing one or more nonpolar functional groups. In the context of the disclosure, a “salt” includes oligonucleotides containing a plurality of cationic binding sites that are saturated by one or more divalent cations (e.g., Ba²⁺, Be²⁺, Ca²⁺, Cu²⁺, Mg²⁺, Mn²⁺, Ni²⁺, or Zn²⁺, or a combination thereof).

The term “therapeutic oligonucleotide” refers to an oligonucleotide that, upon being introduced to (i) a gene of interest encoding a protein or nucleic acid (e.g., RNA) product of interest, reduces or otherwise modulates expression of the protein or nucleic acid product. Examples of therapeutic oligonucleotides are those that attenuate expression of a gene of interest, e.g., by way of the RNA-induced silencing complex (RISC), as described, for example, in Tijsterman and Plasterk, Cell 117(1):1-3 (2004), the disclosure of which is incorporated herein by reference. Additional examples of therapeutic oligonucleotides are those that modulate gene expression by annealing to a gene locus of interest or RNA transcript and: (i) regulate (e.g., inhibit) transcription or translation, (ii) regulate (e.g., induce) exon skipping or inclusion by interfering with a cell's endogenous splicing machinery, and/or (iii) promote gene editing or base editing by way of a CRISPR-associated protein technique known in the art or described herein (e.g., a gRNA). Therapeutic oligonucleotides of the disclosure may be single stranded or double stranded, monomeric or branched. Specific examples of therapeutic oligonucleotides are small interfering RNA (siRNA) molecules, microRNAs (miRNAs), short hairpin RNAs (shRNAs), antisense oligonucleotides (ASOs), and CRISPR guide RNA (gRNA) molecules. In the context of the present disclosure, therapeutic oligonucleotides may be unbound or bound (e.g., conjugated) to one or more additional moieties (e.g., an antibody or other protein).

The term “interfering RNA molecule” refers to an RNA molecule, such as a small interfering RNA (siRNA), microRNA (miRNA), or short hairpin RNA (shRNA), that suppresses the endogenous function of a target RNA transcript.

As used herein, the terms “express” and “expression” refer to one or more of the following events: (1) production of an RNA template from a DNA sequence (e.g., by transcription); (2) processing of an RNA transcript (e.g., by splicing, editing, 5′ cap formation, and/or 3′ end processing); and (3) translation of an RNA into a polypeptide or protein. In the context of a gene that encodes a protein product, the terms “gene expression” and the like are used interchangeably with the terms “protein expression” and the like. Expression of a gene or protein of interest in a patient can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the patient. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the terms “target,” “targeting,” and “targeted,” in the context of the design of an siRNA, refer to generating an antisense strand so as to anneal the antisense strand to a region within the mRNA transcript of interest in a manner that results in a reduction in translation of the mRNA into the protein product.

The term “cationic binding sites” refers to substituents in a therapeutic oligonucleotide that carries either a partial negative charge or a unit negative charge (e.g., the oxyanion of a phosphate or phosphorothioate) and is capable of forming an ionic association with a cation (e.g., a divalent cation).

The term “degree of saturation” refers to the relative proportion of cationic binding sites that are ionically bound by a particular cationic species (e.g., a divalent cation).

The term “hard Lewis acid” refers to a chemical acid that is characterized by a low ionic radius, high positive charge density, strong ability to displace water, and high-energy lowest unoccupied molecular orbital (LUMO).

As used herein, the terms “chemically modified nucleotide,” “nucleotide analog,” “altered nucleotide,” and “modified nucleotide” refer to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function.

As used herein, the term “metabolically stabilized” refers to RNA molecules that contain ribonucleotides that have been chemically modified in order to decrease the rate of metabolism of an RNA molecule that is administered to a subject. Exemplary modifications include 2′-hydroxy to 2′-O-methoxy or 2′-fluoro, and phosphodiester to phosphorothioate.

As used herein, the term “phosphorothioate” refers to a phosphate group of a nucleotide that is modified by substituting one or more of the oxygens of the phosphate group with sulfur.

As used herein, the term “antagomirs” refers to nucleic acids that can function as inhibitors of miRNA activity.

As used herein, the term “gapmers” refers to chimeric antisense nucleic acids that contain a central block of deoxynucleotide monomers sufficiently long to induce RNase H cleavage. The deoxynucleotide block is flanked by ribonucleotide monomers or ribonucleotide monomers containing modifications.

As used herein, the term “mixmers” refers to nucleic acids that contain a mix of locked nucleic acids (LNAs) and DNA.

As used herein, the term “guide RNAs” refers to nucleic acids that have sequence complementarity to a specific sequence in the genome immediately or 1 base pair upstream of the protospacer adjacent motif (PAM) sequence as used in CRISPR/Cas9 gene editing systems. Alternatively, “guide RNAs” may refer to nucleic acids that have sequence complementarity (e.g., are antisense) to a specific messenger RNA (mRNA) sequence. In this context, a guide RNA may also have sequence complementarity to a “passenger RNA” sequence of equal or shorter length, which is identical or substantially identical to the sequence of mRNA to which the guide RNA hybridizes.

As used herein, the term “ethylene glycol chain” refers to a carbon chain with the formula ((CH₂OH)₂).

As used herein, “alkyl” refers to a saturated hydrocarbon group. Alkyl groups may be acyclic or cyclic and contain only C and H when unsubstituted. When an alkyl residue having a specific number of carbons is named, all geometric isomers having that number of carbons are intended to be encompassed and described; thus, for example, “butyl” is meant to include n-butyl, sec-butyl, and iso-butyl. Examples of alkyl include ethyl, propyl, butyl, pentyl, hexyl, heptyl, octyl, cyclopropyl, cyclobutyl, cyclopentyl, cyclohexyl, cycloheptyl, cyclooctyl, and the like. In some embodiments, alkyl may be substituted. Suitable substituents that may be introduced into an alkyl group include, for example, hydroxy, alkoxy, amino, alkylamino, and halo, among others.

As used herein, “alkenyl” refers to an acyclic or cyclic unsaturated hydrocarbon group having at least one site of olefinic unsaturation (i.e., having at least one moiety of the formula C=C). Alkenyl groups contain only C and H when unsubstituted. When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl include —CH═CH₂, —CH₂—CH═CH₂, and —CH₂—CH═CH—CH═CH₂. In some embodiments, alkenyl may be substituted. Suitable substituents that may be introduced into an alkenyl group include, for example, hydroxy, alkoxy, amino, alkylamino, and halo, among others.

As used herein, “alkynyl” refers to an acyclic or cyclic unsaturated hydrocarbon group having at least one site of acetylenic unsaturation (i.e., having at least one moiety of the formula C=C). Alkynyl groups contain only C and H when unsubstituted. When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and tert-pentynyl. Examples of alkynyl include —C≡CH and —C≡C—CH₃. In some embodiments, alkynyl may be substituted. Suitable substituents that may be introduced into an alkynyl group include, for example, hydroxy, alkoxy, amino, alkylamino, and halo, among others.

As used herein the term “phenyl” denotes a monocyclic arene in which one hydrogen atom from a carbon atom of the ring has been removed. A phenyl group may be unsubstituted or substituted with one or more suitable substituents, wherein the substituent replaces an H of the phenyl group.

As used herein, the term “benzyl” refers to monovalent radical obtained when a hydrogen atom attached to the methyl group of toluene is removed. A benzyl generally has the formula of phenyl-CH₂—. A benzyl group may be unsubstituted or substituted with one or more suitable substituents. For example, the substituent may replace an H of the phenyl component and/or an H of the methylene (—CH₂—) component.

As used herein, the term “amide” refers to an alkyl, alkenyl, alkynyl, or aromatic group that is attached to an amino-carbonyl functional group.

As used herein, the terms “internucleoside” and “internucleotide” refer to the bonds between nucleosides and nucleotides, respectively.

As used herein, the term “triazole” refers to heterocyclic compounds with the formula (C₂H₃N₃), having a five-membered ring of two carbons and three nitrogens, the positions of which can change resulting in multiple isomers.

As used herein, the term “terminal group” refers to the group at which a carbon chain or nucleic acid ends.

As used herein, an “amino acid” refers to a molecule containing amine and carboxyl functional groups and a side chain specific to the amino acid.

In some embodiments the amino acid is chosen from the group of proteinogenic amino acids. In some embodiments, the amino acid is an L-amino acid or a D-amino acid. In some embodiments, the amino acid is a synthetic amino acid (e.g., a beta-amino acid).

As used herein, the term “lipophilic amino acid” refers to an amino acid including a hydrophobic moiety (e.g., an alkyl chain or an aromatic ring).

As used herein, the term “target of delivery” refers to the organ or part of the body to which it is desired to deliver the branched oligonucleotide compositions.

As used herein, the term “between X and Y” is inclusive of the values of X and Y. For example, “between X and Y” refers to the range of values between the value of X and the value of Y, as well as the value of X and the value of Y.

As used herein, the term “branched siRNA” refers to a compound containing two or more double-stranded siRNA molecules covalently bound to one another. As an example, branched siRNA molecules may be “di-branched,” also referred to herein as “di-siRNA,” wherein the siRNA molecule includes 2 siRNA molecules covalently bound to one another, e.g., by way of a linker. Branched siRNA molecules may be “tri-branched,” also referred to herein as “tri-siRNA,” wherein the siRNA molecule includes 3 siRNA molecules covalently bound to one another, e.g., by way of a linker. Branched siRNA molecules may be “tetra-branched,” also referred to herein as “tetra-siRNA,” wherein the siRNA molecule includes 4 siRNA molecules covalently bound to one another, e.g., by way of a linker.

As used herein, the term “branch point moiety” refers to a chemical moiety of a branched siRNA structure of the disclosure that may be covalently linked to a 5′ end or a 3′ end of an antisense strand or a sense strand of an siRNA molecule and which may support the attachment of additional single- or double-stranded siRNA molecules. Non-limiting examples of branch point moieties suitable for use in conjunction with the disclosed methods and compositions include, e.g., phosphoroamidite, tosylated solketal, 1,3-diaminopropanol, pentaerythritol, and any one of the branch point moieties described in U.S. Pat. No. 10,478,503.

As used herein, the term “5′ phosphorus stabilizing moiety” refers to a terminal phosphate group that includes phosphates as well as modified phosphates (e.g., phosphorothioates, phosphodiesters, phosphonates). The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′), or alkyl where R′ is H, an amino protecting group, or unsubstituted or substituted alkyl. In some embodiments, the 5′ and or 3′ terminal group may include from 1 to 3 phosphate moieties that are each, independently, unmodified (di- or tri-phosphates) or modified.

It is understood that certain internucleoside linkages provided herein, including, e.g., phosphodiester and phosphorothioate, include a formal charge of −1 at physiological pH, and that said formal charge will be balanced by a cationic moiety, e.g., an alkali metal such as sodium or potassium, an alkali earth metal such as calcium or magnesium, or an ammonium or guanidinium ion, or a plurality of divalent cations (e.g., Ba²⁺, Be²⁺, Ca²⁺, Cu²⁺, Mg²⁺, Mn²⁺, Ni²⁺, Zn²⁺, or a combination thereof).

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 10:117-21, 2000; Rusckowski et al., Antisense Nucleic Acid Drug Dev. 10:333-45, 2000; Stein, Antisense Nucleic Acid Drug Dev. 11:317-25, 2001; Vorobjev et al., Antisense Nucleic Acid Drug Dev. 11:77-85, 2001; and U.S. Pat. No. 5,684,143.

As used herein, the term “complementary” refers to two nucleotides that form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

$$100 multiplied by ( the fraction X / Y )$$

where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “complementarity sufficient to hybridize,” as used herein, refers to a nucleic acid sequence or a portion thereof that need not be fully complementary (e.g., 100% complementary) to a target region or a nucleic acid sequence or a portion thereof that has one or more nucleotide mismatches relative to the target region but that is still capable of hybridizing to the target region under specified conditions. For example, the nucleic acid may be, e.g., 95% complementary, 90%, complementary, 85% complementary, 80% complementary, 75% complementary, 70% complementary, 65% complementary, 60% complementary, 55% complementary, 50% complementary, or less, but still form sufficient base pairs with the target so as to hybridize across its length.

“Hybridization” or “annealing” of nucleic acids is achieved when one or more nucleoside residues within a polynucleotide base pairs with one or more complementary nucleosides to form a stable duplex. The base pairing is typically driven by hydrogen bonding events. Hybridization includes Watson-Crick base pairs formed from natural and/or modified nucleobases. The hybridization can also include non-Watson-Crick base pairs, such as wobble base pairs (guanosine-uracil, hypoxanthine-uracil, hypoxanthine-adenine, and hypoxanthine-cytosine) and Hoogsteen base pairs. Nucleic acids need not be 100% complementary to undergo hybridization. For example, one nucleic acid may be, e.g., 95% complementary, 90%, complementary, 85% complementary, 80% complementary, 75% complementary, 70% complementary, 65% complementary, 60% complementary, 55% complementary, 50% complementary, or less, relative to another nucleic acid, but the two nucleic acids may still form sufficient base pairs with one another so as to hybridize.

The “stable duplex” formed upon the annealing/hybridization of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

The term “gene silencing” refers to the suppression of gene expression, e.g., transgene, heterologous gene and/or endogenous gene expression, which may be mediated through processes that affect transcription and/or through processes that affect post-transcriptional mechanisms. In some embodiments, gene silencing occurs when an RNAi molecule initiates the inhibition or degradation of the mRNA transcribed from a gene of interest in a sequence-specific manner via RNA interference, thereby preventing translation of the gene's product.

The phrase “overactive disease driver gene,” as used herein, refers to a gene having increased activity and/or expression that contributes to or causes a disease state in a subject (e.g., a human). The disease state may be caused or exacerbated by the overactive disease driver gene directly or by way of an intermediate gene(s).

The term “negative regulator,” as used herein, refers to a gene that negatively regulates (e.g., reduces or inhibits) the expression and/or activity of another gene or set of genes (e.g., dysregulated gene or dysregulated gene pathway).

The term “positive regulator,” as used herein, refers to a gene that positively regulates (e.g., increases or saturates) the expression and/or activity of another gene or set of genes (e.g., dysregulated gene or dysregulated gene pathway).

The term “phosphate moiety” as used herein, refers to a terminal phosphate group that includes phosphates as well as modified phosphates. The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′) or alkyl where R′ is H, an amino protecting group or unsubstituted or substituted alkyl. In some embodiments, the 5′ and or 3′ terminal group may include from 1 to 3 phosphate moieties that are each, independently, unmodified (di or tri-phosphates) or modified.

In the context of this disclosure, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring (e.g., modified) portions that function similarly. Such modified or substituted oligonucleotides are often preferred over native forms because of desirable properties such as, for example, enhanced cellular uptake, enhanced affinity for nucleic acid target and increased stability in the presence of nucleases.

As used herein, the terms “subject” and “patient” are used interchangeably and refer to an organism, such as a mammal (e.g., a human), that is suffering from, or is at risk of, a disease, disorder, or condition, as determined by a qualified professional (e.g., a doctor or a nurse practitioner) with or without known in the art laboratory test(s) of sample(s) from the subject.

As used herein, the term “reference subject” refers to a healthy control subject of the same or similar, e.g., age, sex, geographical region, and/or education level as a subject treated with a composition of the disclosure. A healthy reference subject is one that does not suffer from a disease associated with expression of a dysregulated gene or a dysregulated gene pathway. Moreover, a healthy reference subject is one that does not suffer from a disease associated with altered (e.g., increased or decreased) expression and/or activity of a gene.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the term “prion disease” refers to any disease or condition in an organism, the pathogenesis of which involves a prion protein of the organism. Prion diseases include, but are not limited to, Creutzfeldt-Jakob disease, fatal familial insomnia, Gerstmann-Straussler-Scheinker Syndrome, kuru, scrapie, bovine spongiform encephalopathy, and chronic wasting disease. In general, prion diseases are caused by misfolding of the cellular isoform (PrP^{C}) of the prion protein encoded by PRNP. The misfolded protein (PrP^{SC}) triggers the disease. The disease can propagate by PrP^{SC} inducing the misfolding of PrP^{C}.

As used herein, the term “epilepsy” refers to any of a variety of types of epilepsy syndromes, including, but not limited to, frontal lobe epilepsy, occipital lobe epilepsy, medial temporal lobe epilepsy, parietal lobe epilepsy, benign myoclonic epilepsy in infants, juvenile myoclonic epilepsy, childhood absence epilepsy, juvenile absence epilepsy, epilepsy with generalized tonic clonic seizures in childhood, infantile spasms, Lennox-Gastaut syndrome, West syndrome, sleep-related hypermotor epilepsy, progressive myoclonus epilepsies, febrile fits, epilepsy with continuous spike and waves in slow wave sleep, Laudau Kleffner syndrome, Rasmussen's syndrome, epilepsy arising from an inborn error in metabolism, epilepsy of infancy with migrating focal seizures, autosomal dominant nocturnal frontal lobe epilepsy, Ohtahara syndrome, early myoclonic encephalopathy, focal epilepsy, and/or multifocal epilepsy.

As use herein, the term “pain” includes any and all forms of chronic and acute pain, including neuropathic pain and nociceptive pain, among others recited herein.

As used herein, the terms “benefit” and “response” are used interchangeably in the context of a subject undergoing therapy for the treatment of a disease. For example, clinical benefits in the context of a subject administered an siRNA molecule or siRNA composition of the disclosure include, without limitation, a reduction in the duration and/or frequency of symptoms of the disease experienced by the subject, and/or a reduction in disease-associated phenotypes, and/or a reduction in wild type transcripts, mutant transcripts, variant transcripts, or overexpressed transcripts, and/or splice isoforms of transcripts of a target gene.

As used herein, the term “antibody” (Ab) refers to an immunoglobulin molecule that specifically binds to, or is immunologically reactive with, a particular antigen, and includes polyclonal, monoclonal, genetically engineered and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, recombinant IgG (rlgG) fragments, and scFv fragments. Moreover, unless otherwise indicated, the term “monoclonal antibody” (mAb) is meant to include both intact molecules, as well as antibody fragments (such as, for example, Fab and F(ab′)₂ fragments) that are capable of specifically binding to a target protein. Fab and F(ab′)₂ fragments lack the Fc fragment of an intact antibody, clear more rapidly from the circulation of the animal, and may have less non-specific tissue binding than an intact antibody (see Wahl et al., J. Nucl. Med. 24:316, 1983; incorporated herein by reference).

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be, e.g., a Fab, F(ab′)₂, scFv, SMIP, diabody, a triabody, an affibody, a nanobody, an aptamer, or a domain antibody. Examples of binding fragments encompassed by the term “antigen-binding fragment” of an antibody include, but are not limited to: (i) a Fab fragment, a monovalent fragment consisting of the V_{L}, V_{H}, C_{L}, and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

### DETAILED DESCRIPTION

The present disclosure provides compositions and methods for administering therapeutic oligonucleotide molecules to the central nervous system of a subject in the form of a salt containing one or more divalent cations (e.g., Ba²⁺, Be²⁺, Ca²⁺, Cu²⁺, Mg²⁺, Mn²⁺, Ni²⁺, Zn²⁺, or a combination thereof). The therapeutic oligonucleotide molecules may have specific patterns of chemical modifications (e.g., 2′ ribose modifications or internucleoside linkage modifications) to improve resistance against nuclease enzymes, toxicity profile, and physicochemical properties (e.g., thermostability), accompanied with a plurality of divalent cations (e.g., Ba²⁺, Be²⁺, Ca²⁺, Cu²⁺, Mg²⁺, Mn²⁺, Ni²⁺, Zn²⁺, or a combination thereof) saturating pre-existing cationic binding site to further improve the therapeutic oligonucleotide's toxicity profile. In addition, the present disclosure features branched short interfering RNA (siRNA) structures, such as di-branched, tri-branched, and tetra-branched siRNA structures.

### Methods of Therapeutic Oligonucleotide Molecule Synthesis

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. siRNA molecules of the disclosure can be prepared using solution-phase or solid-phase organic synthesis or both.

Further, it is contemplated that for any siRNA agent disclosed herein, further optimization could be achieved by systematically either adding or removing linked nucleosides to generate longer or shorter sequences. Further still, such optimized sequences can be adjusted by, e.g., the introduction of modified nucleosides, and/or modified internucleoside linkages as described herein or as known in the art, including alternative nucleosides, alternative sugar moieties, and/or alternative internucleoside linkages as known in the art and/or discussed herein to further optimize the molecule (e.g., increasing serum stability or circulating half-life, increasing thermal stability, enhancing transmembrane delivery, and/or targeting to a particular location or cell type).

Compositions of therapeutic oligonucleotide molecules of the present disclosure (e.g., siRNA, shRNA, miRNA, gRNA or ASO) may be prepared to include a plurality of cationic binding sites that are saturated by one or more divalent cations (e.g., Ba²⁺, Be²⁺, Ca²⁺, Cu²⁺, Mg²⁺, Mn²⁺, Ni²⁺, or Zn²⁺, or a combination thereof). The compositions may be prepared, for example, by hybridizing the therapeutic oligonucleotide molecule in the presence of the divalent cation. Alternatively, the compositions may be prepared by hybridizing the therapeutic oligonucleotide molecule without the divalent cation, followed by addition of the divalent cation after hybridization. In the case of more than one divalent cation, the divalent cations may be added at the same time or sequentially. For example, the therapeutic oligonucleotide molecule may be hybridized in the presence of two divalent cations. Alternatively, the therapeutic oligonucleotide molecule may be hybridized in the presence of one divalent cation and a second divalent cation is added after hybridization. As a further alternative, the therapeutic oligonucleotide molecule may be hybridized without a divalent cation, followed by the addition of two divalent cations.

### Divalent Cations

The therapeutic oligonucleotide molecules of the disclosure may include a plurality of cationic binding sites (e.g., electron-dense sites) that are saturated by one or more divalent cations (e.g., Ba²⁺, Be²⁺, Ca²⁺, Cu²⁺, Mg²⁺, Mn²⁺, Ni²⁺, or Zn²⁺, or a combination thereof). Because of their positive charge, divalent cations are typically reactive with negatively charges atoms (e.g., oxyanion from a phosphate group or phosphorothioate group carrying a unit or partial negative charge). The present disclosure provides novel evidence that the saturation of cationic binding sites on a therapeutic oligonucleotide molecule with divalent cations significantly reduces toxicity when administered to the CNS of a subject.

The one or more divalent cations may have an ionic radius, when measured in the form of a crystal lattice, of about 30 picometers to about 150 picometers (e.g., from about 30 picometers to about 140 picometers, from about 40 picometers to about 130 picometers, from about 50 picometers to about 120 picometers, from about 60 picometers to about 110 picometers, from about 60 picometers to about 100 picometers, or from about 60 picometers to about 90 picometers). The calculated crystal radii of the divalent cations disclosed by R. D. Shannon, Acta Crystallographica A. 32:751-767, 1976, are herein incorporated by reference.

The degree of saturation of a therapeutic oligonucleotide molecule's cationic binding sites by the one or more divalent cations may range from about 10% to about 100% (e.g., from about 20% to about 100%, from about 30% to about 100%, from about 40% to about 100%, from about 50% to about 100%, from about 60% to about 100%, from about 70% to about 100%, from about 80% to about 100%, or from about 90% to about 100%).

In some embodiments, the antisense strand of the therapeutic oligonucleotide molecule may have a length of from 10 to 30 nucleotides and may be ionically bound to a total of from 10 to 30 divalent cations. For example, the molar ratio of antisense strand nucleotides to divalent cations in the therapeutic oligonucleotide molecule could range from 1:3 to 3:1 (e.g., 1:3, 1.1:3, 1.2:3, 1.3:3, 1.4:3, 1.5:3, 1.6:3, 1.7:3, 1.8:3, 1.9:3, 2:3, 2.1:3, 2.2:3, 2.3:3, 2.4:3, 2.5:3, 2.6:3, 2.7:3, 2.8:3, 2.9:3, 1:1, 3:2.9, 3:2.8, 3:2.7, 3:2.6, 3:2.5, 3:2.4, 3:2.3, 3:2.2, 3:2.1, 3:2, 3:1.9, 3:1.8, 3:1.7, 3:1.6, 3:1.5, 3:1.4, 3:1.3, 3:1.2, 3:1.1, or 3:1).

In some embodiments, the sense strand of the therapeutic oligonucleotide molecule may have a length of from 10 to 30 nucleotides and may be ionically bound to a total of from 10 to 30 divalent cations. For example, the molar ratio of sense strand nucleotides to divalent cations in the therapeutic oligonucleotide molecule could range from 1:3 to 3:1 (e.g., 1:3, 1.1:3, 1.2:3, 1.3:3, 1.4:3, 1.5:3, 1.6:3, 1.7:3, 1.8:3, 1.9:3, 2:3, 2.1:3, 2.2:3, 2.3:3, 2.4:3, 2.5:3, 2.6:3, 2.7:3, 2.8:3, 2.9:3, 1:1, 3:2.9, 3:2.8, 3:2.7, 3:2.6, 3:2.5, 3:2.4, 3:2.3, 3:2.2, 3:2.1, 3:2, 3:1.9, 3:1.8, 3:1.7, 3:1.6, 3:1.5, 3:1.4, 3:1.3, 3:1.2, 3:1.1, or 3:1).

The therapeutic oligonucleotide molecules of the disclosure may be combined with one or more divalent cations in a specific molar ratio. The specific molar ratio of therapeutic oligonucleotide molecule to divalent cation may be relevant to the toxicity benefit achieved by the divalent cation. For example, the molar ratio of therapeutic oligonucleotide molecule to divalent cation may range from 1:10 to 1:50 (e.g., 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40. 1:41, 1:42, 1:43. 1:44, 1:45, 1:46, 1:47, 1:48, 1:49, or 1:50). In some embodiments, the molar ratio of therapeutic oligonucleotide molecule to divalent cation may range from 1:18 to 1:38 (e.g., 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, or 1:38). In some embodiments, the molar ratio of therapeutic oligonucleotide molecule to divalent cation may range from 1:20 to 1:25 (e.g., 1:20, 1:21, 1:22, 1:23, 1:24, or 1:25). In some embodiments, the molar ratio of therapeutic oligonucleotide to divalent cation may be 1:20. In some embodiments, the molar ratio of therapeutic oligonucleotide to divalent cation may be 1:25.

The therapeutic oligonucleotides of the disclosure may be combined with one or more divalent cations in which the divalent cation is present in a specific concentration or range of concentrations. The concentration of the divalent cation may be relevant to the toxicity benefit achieved by the divalent cation. For example, the concentration of the divalent cation may be from 20 mM to 150 mM (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146,147, 148, 149, or 150 mM). In some embodiments, the concentration of the divalent cation is from 20 mM to 100 mM (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM). In some embodiments, the concentration of the divalent cation is from 35 mM to 75 mM (e.g., 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 mM). In some embodiments, the concentration of the divalent cation may be from 40 mM to 70 mM (e.g., 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 mM).

The therapeutic oligonucleotide may include one or more atoms having a negative charge and the divalent cation may include a positive charge. In some embodiments, the therapeutic oligonucleotide and divalent cation are present in an amount so that there is a specific ratio of negative to positive charge present within the composition. Methods of determining the negative to positive charge ratio are known in the art, for example, in Furst et al., Electrophoresis., 37:2685-2691, 2016, the disclosure of which is hereby incorporated by reference. In some embodiments, the ratio of negative charge to positive charge is from 0.75 to 7.5 (e.g., 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5). In some embodiments, the ratio of negative charge to positive charge is from 1.0 to 2.0 (e.g., from 1.0 to 1.9, from 1.0 to 1.8, from 1.0 to 1.7, from 1.0 to 1.6, from 1.0 to 1.5, from 1.0 to 1.4, from 1.0 to 1.3, from 1.0 to 1.2, from 1.0 to 1.1, from 1.1 to 2.0, from 1.2 to 2.0, from 1.3 to 2.0, from 1.4 to 2.0, from 1.5 to 2.0, from 1.6 to 2.0, from 1.7 to 2.0, from 1.8 to 2.0, or from 1.9 to 2.0). In some embodiments, the ratio of negative charge to positive charge is from 0.75 to 6.5 (e.g., from 0.75 to 5.5, from 0.75 to 4.5, from 0.75 to 3.5, from 0.75 to 2.5, from 0.75 to 1.5, or from 0.75 to 1). In some embodiments, the ratio of negative charge to positive charge is from 1 to 7.5 (e.g., from 1.5 to 7.5, from 2.5 to 7.5, from 3.5 to 7.5, from 4.5 to 7.5, from 5.5 to 7.5, or from 6.5 to 7.5).

### Therapeutic Oligonucleotides

The therapeutic oligonucleotides of the disclosure may be in the form of a single-stranded (ss) or double-stranded (ds) RNA structure. In the field of the disclosure, said RNA structure may refer to an siRNA, a short hairpin RNA (shRNA), a microRNA (miRNA), a CRISPR guide RNA (gRNA), or an oligonucleotide (ASO). In some embodiments, the siRNA molecule may be a di-branched, tri-branched, or tetra-branched molecule. The therapeutic oligonucleotides of the disclosure may contain one or more phosphodiester internucleoside linkages and/or an analog thereof, such as a phosphorothioate internucleoside linkage, in which oxyanion moieties are electrostatically neutralized by ionic bonding to a divalent metal cation, such as Ba²⁺, Be²⁺, Ca²⁺, Cu²⁺, Mg²⁺, Mn²⁺, Ni²⁺, or Zn²⁺.

siRNA Structure

The siRNA molecules of the disclosure may be in the form of a single-stranded (ss) or double-stranded (ds) RNA structure. In some embodiments, the siRNA molecules may be di-branched, tri-branched, or tetra-branched molecules. Furthermore, the siRNA molecules of the disclosure may contain one or more phosphodiester internucleoside linkages and/or an analog thereof, such as a phosphorothioate internucleoside linkage. The siRNA molecules of the disclosure may further contain chemically modified nucleosides having 2′ sugar modifications.

The simplest siRNAs consist of a ribonucleic acid, including a ss- or ds-structure, formed by a first strand (i.e., antisense strand), and in the case of a ds-siRNA, a second strand (i.e., sense strand). The first strand includes a stretch of contiguous nucleotides that is at least partially complementary to a target nucleic acid. The second strand also includes a stretch of contiguous nucleotides where the second stretch is at least partially identical to a target nucleic acid. The first strand and said second strand may be hybridized to each other to form a double-stranded structure. The hybridization typically occurs by Watson Crick base pairing.

Depending on the sequence of the first and second strand, the hybridization or base pairing is not necessarily complete or perfect, which means that the first and second strand are not 100% base-paired due to mismatches. One or more mismatches may also be present within the duplex without necessarily impacting the siRNA RNA interference (RNAi) activity.

The first strand contains a stretch of contiguous nucleotides which is essentially complementary to a target nucleic acid. Typically, the target nucleic acid sequence is, in accordance with the mode of action of interfering ribonucleic acids, a ss-RNA, preferably an mRNA. Such hybridization occurs most likely through Watson Crick base pairing but is not necessarily limited thereto. The extent to which the first strand has a complementary stretch of contiguous nucleotides to a target nucleic acid sequence may be between 80% and 100%, e.g., 80%, 85%, 90%, 95%, or 100% complementary.

siRNAs described herein may employ modifications to the nucleobase, phosphate backbone, ribose core, 5′- and 3′-ends, and branching, wherein multiple strands of siRNA may be covalently linked.

### Lengths of Therapeutic Oligonucleotides

It is within the scope of the disclosure that any length, known and previously unknown in the art, may be employed for the current invention. As described herein, potential lengths for an antisense strand of the therapeutic oligonucleotide of the present disclosure is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the sense strand of the therapeutic oligonucleotide of the present disclosure is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

### 2′ Sugar Modifications

The present disclosure includes ss- and ds-RNA interfering molecule compositions (e.g., siRNA, shRNA, miRNA, gRNA or ASO) including at least one (e.g., at least 1, 2, 3, 4, 5, 6, 7, 8, 9, 10, 11, or more) nucleosides having 2′ sugar modifications. Possible 2′-modifications include all possible orientations of OH; F; O-, S-, or N-alkyl; O-, S-, or N-alkenyl; O—, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modification includes a 2′-O-methyl (2′-O-Me) modification. Some embodiments use O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other potential sugar substituent groups include: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a group for improving the pharmacokinetic properties of an oligonucleotide, or a group for improving the pharmacodynamic properties of an oligonucleotide, and other substituents having similar properties. In some embodiments, the modification includes 2′-methoxyethoxy (2′-O—CH₂CH₂OCH₃, also known as 2′-O-(2-methoxyethyl) or 2′-MOE). In some embodiments, the modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the therapeutic oligonucleotide, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Nucleobase Modifications

Therapeutic oligonucleotides may also include nucleosides or other surrogate or mimetic monomeric subunits that include a nucleobase (often referred to in the art simply as “base” or “heterocyclic base moiety”). The nucleobase is another moiety that has been extensively modified or substituted and such modified and or substituted nucleobases are amenable to the present disclosure. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in Kroschwitz, J. I., ed. The Concise Encyclopedia of Polymer Science and Engineering, New York, John Wiley & Sons, 1990, pp. 858-859; those disclosed by Englisch et al., Angewandte Chemie, International Edition 30:613, 1991; and those disclosed by Sanghvi, Y. S., Chapter 16, Antisense Research and Applications, CRC Press, Gait, M. J. ed., 1993, pp. 289-302. Therapeutic oligonucleotides of the present disclosure may also include polycyclic heterocyclic compounds in place of one or more heterocyclic base moieties. A number of tricyclic heterocyclic compounds have been previously reported. These compounds are routinely used in antisense applications to increase the binding properties of the modified strand to a target strand.

Representative cytosine analogs that make three hydrogen bonds with a guanosine in a second strand include 1,3-diazaphenoxazine-2-one (Kurchavov et al., Nucleosides and Nucleotides, 16:1837-46, 1997), 1,3-diazaphenothiazine-2-one (Lin et al. Am. Chem. Soc., 117:3873-4, 1995), and 6,7,8,9-tetrafluoro-I,3-diazaphenoxazine-2-one (Wang et al., Tetrahedron Lett., 39:8385-8, 1998). Incorporated into oligonucleotides, these base modifications were shown to hybridize with complementary guanine and the latter was also shown to hybridize with adenine and to enhance helical thermal stability by extended stacking interactions (also see U.S. Ser. No. 10/155,920 and U.S. Ser. No. 10/013,295, both of which are herein incorporated by reference in their entirety). Further helix-stabilizing properties have been observed when a cytosine analog/substitute has an aminoethoxy moiety attached to the rigid 1,3-diazaphenoxazine-2-one scaffold (Lin et al., Am. Chem. Soc., 120:8531-2, 1998).

### Internucleoside Linkage Modifications

Another variable in the design of the present disclosure is the internucleoside linkage making up the phosphate backbone of the therapeutic oligonucleotide. Although the natural RNA phosphate backbone may be employed here, derivatives thereof may be used which enhance desirable characteristics of the therapeutic oligonucleotide. Although not limiting, of particular importance in the present disclosure is protecting parts, or the whole, of the therapeutic oligonucleotide from hydrolysis. One example of a modification that decreases the rate of hydrolysis is phosphorothioates. Any portion or the whole of the backbone may contain phosphate substitutions (e.g., phosphorothioates, phosphodiesters, etc.). For instance, the internucleoside linkages may be between 0 and 100% phosphorothioate, e.g., between 0 and 100%, 10 and 100%, 20 and 100%, 30 and 100%, 40 and 100%, 50 and 100%, 60 and 100%, 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphorothioate linkages. Similarly, the internucleoside linkages may be between 0 and 100% phosphodiester linkages, e.g., between 0 and 100%, 10 and 100%, 20 and 100%, 30 and 100%, 40 and 100%, 50 and 100%, 60 and 100% 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphodiester linkages.

Specific examples of some potential therapeutic oligonucleotides useful in this invention include oligonucleotides containing modified e.g., non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides. A preferred phosphorus containing modified internucleoside linkage is the phosphorothioate internucleoside linkage. In some embodiments, the modified oligonucleotide backbones containing a phosphorus atom therein include, for example, phosphorothioates, phosphorodithioates, phosphotriesters, aminoalkylphosphotriesters, methyl and other alkyl phosphonates including 3-alkylene phosphonates, 5′-alkylene phosphonates, phosphinates, phosphoramidates including 3-amino phosphoramidate and aminoalkylphosphoramidates, thionophosphoramidates, thionoalkylphosphonates, thionoalkylphosphotriesters, selenophosphates, and boranophosphates having normal 3-5′ linkages, 2′-5′ linked analogs of these, and those having inverted polarity wherein one or more internucleotide linkages is a 3′ to 3′, 5′ to 5′ or 2′ to 2′ linkage. Exemplary U.S. patents describing the preparation of phosphorus-containing linkages include but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. No. RE39,464, the entire contents of each of which are hereby incorporated herein by reference.

In some embodiments, the modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Non-limiting examples of U.S. patents that teach the preparation of non-phosphorus backbones include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

Patterns of Modifications of siRNA Molecules

Therapeutic oligonucleotides of the disclosure (e.g., siRNA molecules) may have various patterns of chemically modified residues, such as those described in this section. Nucleosides used in the disclosure tolerate a range of modifications in the nucleobase and sugar. A complete therapeutic oligonucleotide (e.g., siRNA molecules), single-stranded or double-stranded, may have 1, 2, 3, 4, 5, or more different nucleosides that each appear in the RNA strand or strands once or more. The nucleosides may appear in a repeating pattern (e.g., alternating between two modified nucleosides) or may be a strand of one type of nucleoside with substitutions of a second type of nucleoside. Similarly, internucleoside linkages may be of one or more type appearing in a single- or double-stranded siRNA in a repeating pattern (e.g., alternating between two internucleoside linkages) or may be a strand of one type of internucleoside linkage with substitutions of a second type of internucleoside linkage. Though the therapeutic oligonucleotides of the disclosure may tolerate a range of substitution patterns, the following exemplify some preferred patterns in which A and B represent nucleosides of two types, and T and P represent internucleoside linkages of two types:

### Pattern 1:

### A-T-B-T-A-P-A-P-A-P-A-P-A-P-A-P-A-P-A-P-A-P-A-P-A-P—B-T-A-T-A-T-A-T-A-T-A-T-A-T

### A-T-A-T-A-P-A-P-A-P-A-P-A-P-A-P-A-P-A-P-A-P-A-P-A-P-A-T-A-T

### Pattern 2:

### A-T-A-T-A-P—B—P—B—P—B—P-A-P-A-P-A-P-A-P-A-P-A-P-A-P—B-T-A-T-A-T-A-T-A-T-A-T-A-T

### A-T-A-T-A-P-A-P-A-P-A-P-A-P-A-P-A-P-A-P-A-P-A-P-A-P-A-T-A-T

### Pattern 3:

### A-T-B-T-A-P—B—P—B—P—B—P-A-P-A-P-A-P-A-P-A-P-A-P-A-P—B-T-A-T-A-T-A-T-A-T-A-T-A-T

### A-T-A-T-A-P-A-P-A-P-A-P-A-P-A-P-A-P-A-P-A-P-A-P-A-P-A-T-A-T

### Pattern 4:

### A-T-B-T-A-P-A-P-A-P-A-P-A-P-A-P-A-P-A-P-A-P-A-P-A-P—B-T-A-T-A-T-A-T-A-T-A-T-A-T

### A-T-A-T-A-P-A-P-A-P-A-P—B—P-A-P-A-P—B—P—B—P-A-P-A-P-A-T-A-T

### Pattern 5:

### A-T-B-T-A-P-A-P-A-P—B—P-A-P-A-P-A-P-A-P-A-P-A-P-A-P—B-T-A-T-B-T-A-T-A-T-A-T-A-T

### A-T-A-T-A-P-A-P-A-P-A-P—B—P-A-P—B—P—B—P—B—P-A-P-A-P-A-T-A-T.

In some embodiments, T represents phosphorothioate, and P represents phosphodiester.

In some embodiments, the siRNA molecule of the disclosure features any one of the siRNA nucleotide modification patterns and/or internucleoside linkage modification patterns described in International Patent Application Publication Nos. WO 2016/161388 and WO 2020/041769, the disclosures of which are incorporated in their entirety herein.

The following section provides a further set of exemplary scaffolds into which the siRNA molecules of the disclosure may be incorporated.

In some embodiments of the disclosure, the siRNA may contain an antisense strand including a region represented by Formula I, wherein Formula I is, in the 5′-to-3′ direction

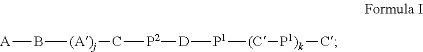

wherein A is represented by the formula C—P¹-D-P¹; each A′ is represented by the formula C—P²-D-P²; B is represented by the formula C—P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments, the antisense strand includes a structure represented by Formula A1, wherein Formula A1 is, in the 5′-to-3′ direction:

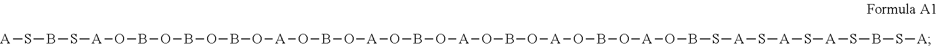

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula II, wherein Formula II is, in the 5′-to-3′ direction:

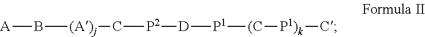

wherein A is represented by the formula C—P¹-D-P¹; each A′ is represented by the formula C—P²-D-P²; B is represented by the formula C—P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments of the disclosure, the antisense strand includes a structure represented by Formula A2, wherein Formula A2 is, in the 5′-to-3′ direction:

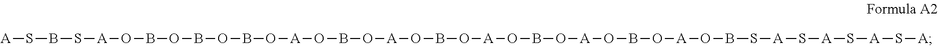

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula III, wherein Formula III is, in the 5′-to-3′ direction:

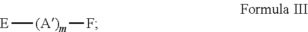

wherein E is represented by the formula (C—P¹)₂; F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D; A′, C, D, P¹, and P² are as defined in Formula I; and m is an integer from 1 to 7 (e.g., 1, 2, 3, 4, 5, 6, or 7). In some embodiments, m is 4. The sense strand is complementary (e.g., fully or partially complementary) to the antisense strand.

In some embodiments of the disclosure, the sense strand includes a structure represented by Formula S1, wherein Formula S1 is, in the 5′-to-3′ direction:

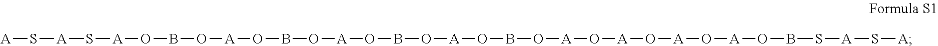

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S2, wherein Formula S2 is, in the 5′-to-3′ direction:

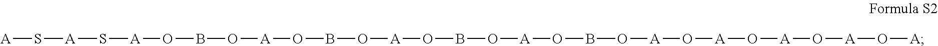

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S3, wherein Formula S3 is, in the 5′-to-3′ direction:

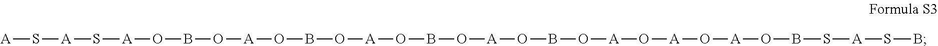

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S4, wherein Formula S4 is, in the 5′-to-3′ direction:

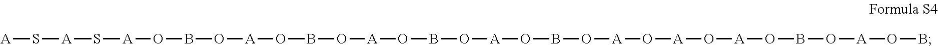

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula IV, wherein Formula IV is, in the 5′-to-3′ direction:

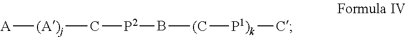

wherein A is represented by the formula C—P¹-D-P¹; each A′ is represented by the formula C—P²-D-P²; B is represented by the formula D-P¹—C—P¹-D-P¹; each C is a 2′-O-Me ribonucleoside; each C′, independently, is a 2′-O-Me ribonucleoside or a 2′-F ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 6. In some embodiments, k is 4. In some embodiments, j is 6 and k is 4. The antisense strand is complementary (e.g., fully or partially complementary) to a target nucleic acid.

In some embodiments of the disclosure, the antisense strand includes a structure represented by Formula A3, wherein Formula A3 is, in the 5′-to-3′ direction:

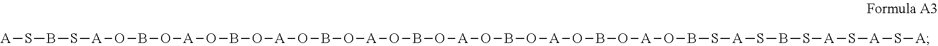

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA of the disclosure may have a sense strand represented by Formula V, wherein Formula V is, in the 5′-to-3′ direction:

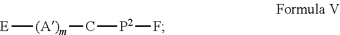

wherein E is represented by the formula (C—P¹)₂; F is represented by the formula D-P¹—C—P¹—C, D-P²—C—P²—C, D-P¹—C—P¹-D, or D-P²—C—P²-D; A′, C, D, P¹, and P² are as defined in Formula IV; and m is an integer from 1 to 7 (e.g., 1, 2, 3, 4, 5, 6, or 7). In some embodiments, m is 5. The sense strand is complementary (e.g., fully or partially complementary) to the antisense strand.

In some embodiments of the disclosure, the sense strand includes a structure represented by Formula S5, wherein Formula S5 is, in the 5′-to-3′ direction:

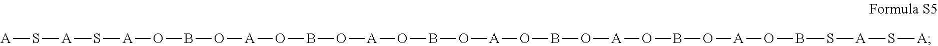

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S6, wherein Formula S6 is, in the 5′-to-3′ direction:

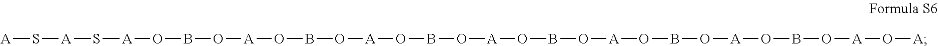

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S7, wherein Formula S7 is, in the 5′-to-3′ direction:

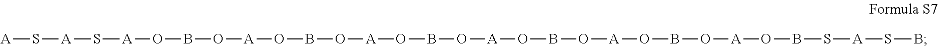

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S8, wherein Formula S8 is, in the 5′-to-3′ direction:

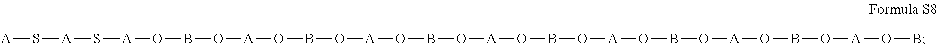

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula VI, wherein Formula VI is, in the 5′-to-3′ direction:

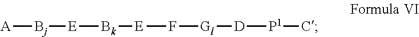

wherein A is represented by the formula C—P¹-D-P¹; each B is represented by the formula C—P²; each C is a 2′-O-Me ribonucleoside; each C′, independently, is a 2′-O-Me ribonucleoside or a 2′-F ribonucleoside; each D is a 2′-F ribonucleoside; each E is represented by the formula D-P²—C—P²; F is represented by the formula D-P¹—C—P¹; each G is represented by the formula C—P¹; each P¹ is a phosphorothioate internucleoside linkage; each P² is a phosphodiester internucleoside linkage; j is an integer from 1 to 7 (e.g., 1, 2, 3, 4, 5, 6, or 7); k is an integer from 1 to 7 (e.g., 1, 2, 3, 4, 5, 6, or 7); and l is an integer from 1 to 7 (e.g., 1, 2, 3, 4, 5, 6, or 7). In some embodiments, j is 3. In some embodiments, k is 6. In some embodiments, I is 2. In some embodiments, j is 3, k is 6, and I is 2. The antisense strand is complementary (e.g., fully or partially complementary) to a target nucleic acid.

In some embodiments of the disclosure, the antisense strand includes a structure represented by Formula A4, wherein Formula A4 is, in the 5′-to-3′ direction:

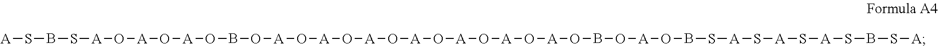

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain a sense strand including a region represented by Formula VII, wherein Formula VII is, in the 5′-to-3′ direction:

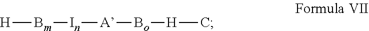

wherein A′ is represented by the formula C—P²-D-P²; each H is represented by the formula (C—P¹)₂; each I is represented by the formula (D-P²); B, C, D, P¹, and P² are as defined in Formula VI; m is an integer from 1 to 7 (e.g., 1, 2, 3, 4, 5, 6, or 7); n is an integer from 1 to 7 (e.g., 1, 2, 3, 4, 5, 6, or 7); and o is an integer from 1 to 7 (e.g., 1, 2, 3, 4, 5, 6, or 7). In some embodiments, m is 3. In some embodiments, n is 3. In some embodiments, o is 3. In some embodiments, m is 3, n is 3, and o is 3. The sense strand is complementary (e.g., fully or partially complementary) to the antisense strand.

In some embodiments of the disclosure, the sense strand includes a structure represented by Formula S9, wherein Formula S9 is, in the 5′-to-3′ direction:

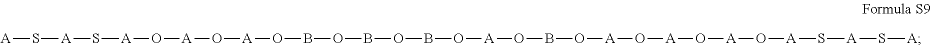

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region that is represented by Formula VIII:

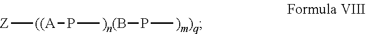

wherein Z is a 5′ phosphorus stabilizing moiety; each A is a 2′-O-methyl (2′-O-Me) ribonucleoside; each B is a 2′-fluoro-ribonucleoside; each P is, independently, an internucleoside linkage selected from a phosphodiester linkage and a phosphorothioate linkage; n is an integer from 1 to 5 (e.g., 1, 2, 3, 4, or 5); m is an integer from 1 to 5 (e.g., 1, 2, 3, 4, or 5); and q is an integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).

### 5′ Phosphorus Stabilizing Moiety

To further protect the therapeutic oligonucleotides of this disclosure from degradation, a 5′-phosphorus stabilizing moiety may be employed. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each interfering RNA strand may independently and optionally employ any suitable 5′-phosphorus stabilizing moiety.

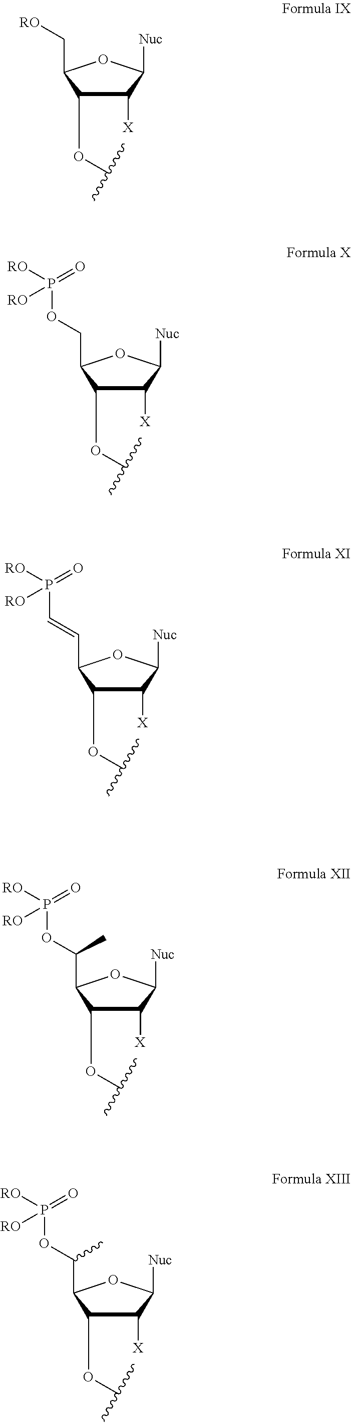

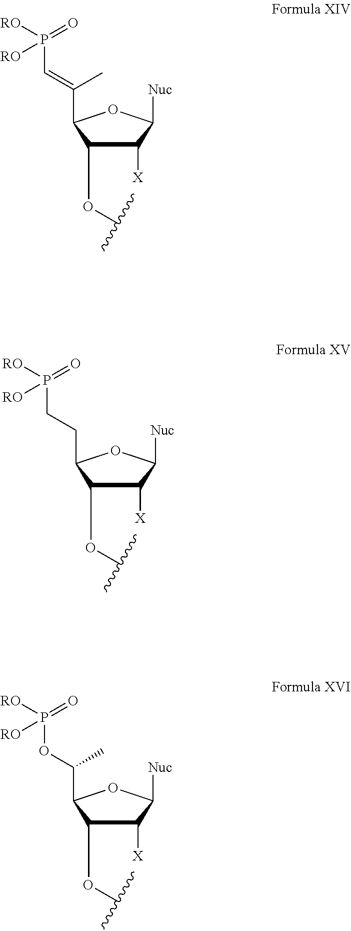

Some exemplary endcaps are demonstrated in Formulas IX-XVI. Nuc in Formulas IX-XVI represents a nucleobase or nucleobase derivative or replacement as described herein. X in formula IX-XVI represents a 2′-modification as described herein. Some embodiments employ hydroxy as in Formula XIV, phosphate as in Formula XV, vinylphosphonates as in Formula XVI and XIX, 5′-methyl-substitued phosphates as in Formula XVII, XIX, and XXI, or methylenephosphonates as in Formula XX. Vinyl 5′-vinylphsophonate as a 5′-phosphorus stabilizing moiety as demonstrated in Formula XVI.

### Hydrophobic Moieties

The present disclosure further provides therapeutic oligonucleotides having one or more hydrophobic moieties attached thereto. The hydrophobic moiety may be covalently attached to the 5′ end or the 3′ end of the therapeutic oligonucleotides of the disclosure. Non-limiting examples of hydrophobic moieties suitable for use with the therapeutic oligonucleotides of the disclosure may include cholesterol, vitamin D, tocopherol, phosphatidylcholine (PC), docohexaenoic acid, docosanoic acid, PC-docosanoic acid, eicosapentaenoic acid, lithocholic acid or any combination of the aforementioned hydrophobic moieties with PC.

### Interfering RNA Branching

The therapeutic oligonucleotides of the disclosure may be branched. For example, the siRNA molecules of the disclosure may have one of several branching patterns, as described herein.

According to the present disclosure, the siRNA molecules disclosed herein may be branched siRNA molecules. The siRNA molecule may not be branched, or may be di-branched, tri-branched, or tetra-branched, connected through a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 1.

TABLE 1
Branched siRNA structures

| Di-branched | Tri-branched | Tetra-branched |
|---|---|---|
| 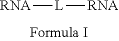 | 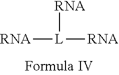 | 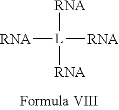 |
| 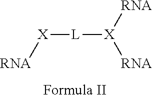 | 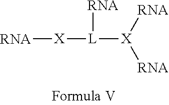 | 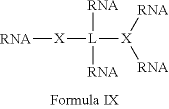 |
| 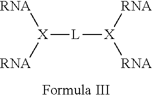 | 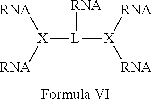 | 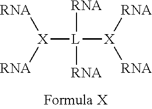 |
| | 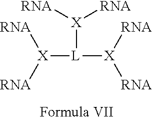 | 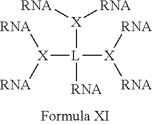 |
| | | 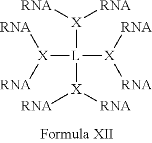 |

In some embodiments, the siRNA molecule is a branched siRNA molecule. In some embodiments, the branched siRNA molecule is di-branched, tri-branched, or tetra-branched. In some embodiments, the di-branched siRNA molecule is represented by any one of Formulas I-III, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

In some embodiments, the tri-branched siRNA molecule represented by any one of Formulas IV-VII, wherein each RNA, independently, is an siRNA molecule, L is a linker, and each X, independently, represents a branch point moiety.

In some embodiments, the tetra-branched siRNA molecule represented by any one of Formulas VIII-XII, wherein each RNA, independently, is an siRNA molecule, L is a linker, and each X, independently, represents a branch point moiety.

### Linkers

Multiple strands of siRNA described herein may be covalently attached by way of a linker. The effect of this branching improves, inter alia, cell permeability allowing better access into cells (e.g., neurons or glial cells) in the CNS. Any linking moiety may be employed which is not incompatible with the siRNAs of the present invention. Linkers include ethylene glycol chains of 2 to 10 subunits (e.g., 2, 3, 4, 5, 6, 7, 8, 9, or 10 subunits), alkyl chains, carbohydrate chains, block copolymers, peptides, RNA, DNA, and others. In some embodiments, any carbon or oxygen atom of the linker is optionally replaced with a nitrogen atom, bears a hydroxyl substituent, or bears an oxo substituent. In some embodiments, the linker is a poly-ethylene glycol (PEG) linker. The PEG linkers suitable for use with the disclosed compositions and methods include linear or non-linear PEG linkers. Examples of non-linear PEG linkers include branched PEGs, linear forked PEGs, or branched forked PEGs.

PEG linkers of various weights may be used with the disclosed compositions and methods. For example, the PEG linker may have a weight that is between 5 and 500 Daltons. In some embodiments, a PEG linker having a weight that is between 500 and 1,000 Dalton may be used. In some embodiments, a PEG linker having a weight that is between 1,000 and 10,000 Dalton may be used. In some embodiments, a PEG linker having a weight that is between 200 and 20,000 Dalton may be used. In some embodiments, the linker is covalently attached to a sense strand of the siRNA. In some embodiments, the linker is covalently attached to an antisense strand of the siRNA. In some embodiments, the PEG linker is a triethylene glycol (TrEG) linker. In some embodiments, the PEG linker is a tetraethylene linker (TEG).

In some embodiments, the linker is an alkyl chain linker. In some embodiments, the linker is a peptide linker. In some embodiments, the linker is an RNA linker. In some embodiments, the linker is a DNA linker.

Linkers may covalently link 2, 3, 4, or 5 unique siRNA strands. The linker may covalently bind to any part of the siRNA oligomer. In some embodiments, the linker attaches to the 3′ end of nucleosides of each siRNA strand. In some embodiments, the linker attaches to the 5′ end of nucleosides of each siRNA strand. In some embodiments, the linker attaches to a nucleoside of an siRNA strand (e.g., sense or antisense strand) by way of a covalent bond-forming moiety. In some embodiments, the covalent-bond-forming moiety is selected from the group consisting of an alkyl, ester, amide, carbonate, carbamate, triazole, urea, formacetal, phosphonate, phosphate, and phosphate derivative (e.g., phosphorothioate, phosphoramidate, etc.).

In some embodiments, the linker has a structure of Formula L1:

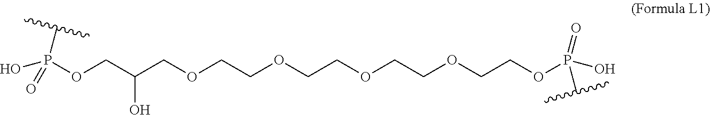 [SMILES: *P(=O)(O)OCCOCCOCCOCCOCC(O)COP(*)(=O)O]

In some embodiments, the linker has a structure of Formula L2:

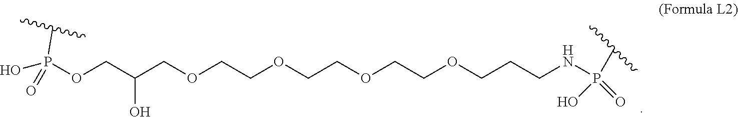 [SMILES: *P(=O)(O)NCCCOCCOCCOCCOCC(O)COP(*)(=O)O]

In some embodiments, the linker has a structure of Formula L3:

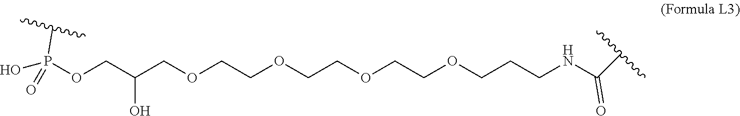 [SMILES: *C(=O)NCCCOCCOCCOCCOCC(O)COP(*)(=O)O]

In some embodiments, the linker has a structure of Formula L4:

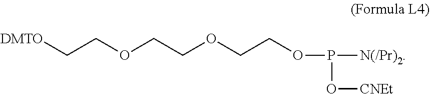

In some embodiments, the linker has a structure of Formula L5:

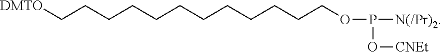

In some embodiments, the linker has a structure of Formula L6:

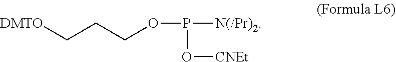

In some embodiments, the linker has a structure of Formula L7, as is shown below:

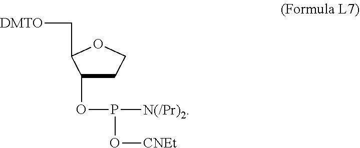

In some embodiments, the linker has a structure of Formula L8:

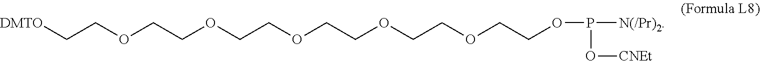

In some embodiments, the linker has a structure of Formula L9:

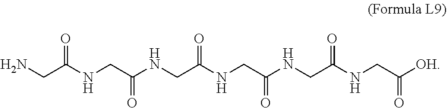 [SMILES: NCC(=O)NCC(=O)NCC(=O)NCC(=O)NCC(=O)NCC(=O)O]

In some embodiments, the selection of a linker for use with one or more of the branched siRNA molecules disclosed herein may be based on the hydrophobicity of the linker, such that, e.g., desirable hydrophobicity is achieved for the one or more branched siRNA molecules of the disclosure. For example, a linker containing an alkyl chain may be used to increase the hydrophobicity of the branched siRNA molecule as compared to a branched siRNA molecule having a less hydrophobic linker or a hydrophilic linker.

The siRNA agents disclosed herein may be synthesized and/or modified by methods well established in the art, such as those described in Beaucage, S. L. et al. (edrs.), Current Protocols in Nucleic Acid Chemistry, John Wiley & Sons, Inc., New York, N.Y., 2000, which is hereby incorporated herein by reference.

### Methods of Treatment

The disclosure provides methods of treating a subject in need of gene silencing. The gene silencing may be performed in order to silence defective or overactive genes, silence negative regulators of genes with reduced expression, silence wild type genes with an activating role in a pathway(s) that increases activity of a disease driver gene, silence splice isoforms of a gene(s) that, when selectively knocked down, may elevate total expression of the gene(s), among other reasons, so long as the goal is to restore genetic and biochemical pathway activity from a disease state towards a healthy state. The method may include delivering to the CNS of the subject (e.g., a human) the therapeutic oligonucleotides of the disclosure or a pharmaceutical composition containing the same by any appropriate route of administration (e.g., intrastriatal, intracerebroventricular, intrathecal injection, or by intra-cisterna magna injection by catheterization). The active compound can be administered in any suitable dose. The actual dosage amount of a composition of the present disclosure administered to a patient can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary. Subjects may be adult or pediatric humans, with or without comorbid diseases.

### Indications

The subject in need of gene silencing may be in need of silencing of a gene found in the CNS (e.g., in a microglial cell). The gene may be associated with a specific disease or disorder. For example, the gene may be associated with Huntington's disease, Parkinson's disease, Alzheimer's disease, amyotrophic lateral sclerosis (ALS), dementia with Lewy bodies (DLB), pure autonomic failure, Lewy body dysphagia, incidental Lewy body disease (ILBD), inherited Lewy body disease, olivopontocerebellar atrophy (OPCA), striatonigral degeneration, Shy-Drager syndrome, epilepsy or an epilepsy disorder, a prion disease, or pain or a pain disorder.

Target Genes The methods of gene silencing described herein may be performed in order to silence defective or overactive genes, silence negative regulators of genes with reduced expression, silence wild type genes with an activating role in a pathway(s) that increases activity of a disease driver gene, silence splice isoforms of a gene(s) that, when selectively knocked down, may elevate total expression of the gene(s), among other reasons, so long as the goal is to restore genetic and biochemical pathway activity from a disease state towards a healthy state.

The disease or disorder may be associated with any of the following genes: ABCA7, ABI3, ADAM10, APOC1, APOE, AXL, BIN1, C1QA, C3, C90RF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, HTT, IFIT1, IFIT3, IFITM3, IFNAR1, IFNAR2, IGF1, IL10RA, IL1A, IL1B, IL1RAP, INPP5D, ITGAM, ITGAX, KCNT1, LILRB4, LPL, MAPT, MEF2C, MMP12, MS4A4A, MS4A6A, MSH3, NLRP3, NME8, NOS2, PICALM, PILRA, PLCG2, PRNP, PTK2B, SCIMP, SCN9A, SLC24A4, SNCA, SORL1, SPI1, SPP1, SPPL2A, TBK1, TNF, TREM2, TREML2, TYROBP, and ZCWPW1. In some embodiments, the disease or disorder is associated with any of the following genes: APOE, BIN1, C1QA, C3, C90RF72, CCL5, CD33, CLU/APOJ, CR1, CXCL10, CXCL13, IFIT1, IFIT3, IFITM3, IFNAR1, IFNAR2, IL10RA, IL1A, IL1B, IL1 RAP, INPP5D, ITGAM, MEF2C, MMP12, NLRP3, NOS2, PILRA, PLCG2, PTK2B, SLC24A4, TBK1, and TNF. In some embodiments, the disease or disorder is associated with any of the following genes: HTT, MAPT, SNCA, C90RF72, APOE, SCN9A, KCNT1, PRNP, and MSH3. In some embodiments, the disease or disorder is associated with an HTT gene. In some embodiments, the disease or disorder is associated with a MAPT gene. In some embodiments, the disease or disorder is associated with an SNCA gene. In some embodiments, the disease or disorder is associated with a C90RF72 gene. In some embodiments, the disease or disorder is associated with an APOE gene. In some embodiments, the disease or disorder is associated with an SCN9A gene. In some embodiments, the disease or disorder is associated with a KCNT1 gene. In some embodiments, the disease or disorder is associated with a PRNP gene. In some embodiments, the disease or disorder is associated with an MSH3 gene.

### Osmolality

Administration of a therapeutic oligonucleotide of the disclosure may influence the osmolality of a subject (e.g., of cerebrospinal fluid (CSF)). CSF osmolality of subjects being treated with a therapeutic oligonucleotide of the disclosure may be, for example, from 250 to 450 mOsmol/kg. In some embodiments, the CSF osmolality is from 250 to 350 mOsmol/kg. The CSF osmolality of the subject may be affected by the concentration of the divalent cation. A person overseeing treatment of a subject may be able to monitor the CSF osmolality of the subject and adjust the dosage accordingly. For example, the dose can be decreased in a subject exhibiting a higher-than-normal osmolality.

Alternatively, the concentration of sodium ions in the composition containing the therapeutic oligonucleotide can be altered. For example, in a liquid formulation of a therapeutic oligonucleotide, the concentration of sodium may be modulated to increase or decrease the resulting osmolality, without having a negative effect on the toxicity benefit of the divalent cation. Reducing the level of sodium in a formulation may allow for the maintenance of normal physiological osmolality levels in subjects undergoing treatment with a therapeutic oligonucleotide of the disclosure.

### Pharmaceutical Compositions

The therapeutic oligonucleotides in the present disclosure may be formulated into a pharmaceutical composition for administration to a subject in a biologically compatible form suitable for administration in vivo. Accordingly, the present disclosure provides a pharmaceutical composition containing a therapeutic oligonucleotide of the disclosure in admixture with a suitable diluent, carrier, or excipient. The therapeutic oligonucleotides may be administered, for example, directly into the CNS of the subject (e.g., by way of intrastriatal, intracerebroventricular, intrathecal injection or by intra-cisterna magna injection by catheterization).

Conventional procedures and ingredients for the selection and preparation of suitable formulations are described, for example, in Remington, J. P. The Science and Practice of Pharmacy, Easton, PA. Mack Publishers, 2012, 22^{nd} ed. And in The United States Pharmacopeial Convention, The National Formulary, United States Pharmacopeial, 2015, USP 38 NF 33).

Under ordinary conditions of storage and use, a pharmaceutical composition may contain a preservative, e.g., to prevent the growth of microorganisms. Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a subject in need of treatment.

A pharmaceutical composition may be administered to a subject, e.g., a human subject, alone or in combination with pharmaceutically acceptable carriers, as noted herein, the proportion of which may be determined by the solubility and/or chemical nature of the compound, chosen route of administration, and standard pharmaceutical practice.

### Dosing Regimens

A physician having ordinary skill in the art can readily determine an effective amount of the therapeutic oligonucleotide (e.g., siRNA, shRNA, miRNA, gRNA or ASO) for administration to a mammalian subject (e.g., a human) in need thereof. For example, a physician could start prescribing doses of one of the therapeutic oligonucleotides of the disclosure at levels lower than that required in order to achieve the desired therapeutic effect and gradually increase the dosage until the desired effect is achieved. Alternatively, a physician may begin a treatment regimen by administering the one of the therapeutic oligonucleotides of the disclosure at a high dose and subsequently administer progressively lower doses until a therapeutic effect is achieved (e.g., a reduction in expression of a target gene sequence). In general, a suitable daily dose of one of the therapeutic oligonucleotides of the disclosure will be an amount of the therapeutic oligonucleotide (e.g., siRNA) which is the lowest dose effective to produce a therapeutic effect. The ss- or ds-therapeutic oligonucleotides of the disclosure may be administered by injection, e.g., intrathecally, intracerebroventricularly, intrastriatally or by intra-cisterna magna injection by catheterization (e.g., injection into the caudate nucleus or putamen). A daily dose of a therapeutic composition of the therapeutic oligonucleotides of the disclosure may be administered as a single dose or as two, three, four, five, six or more doses administered separately at appropriate intervals throughout the day, week, month, or year, optionally, in unit dosage forms. While it is possible for the therapeutic oligonucleotides of the disclosure to be administered alone, it may also be administered as a pharmaceutical formulation in combination with excipients, carriers, and optionally, additional therapeutic agents.

### Routes of Administration

The method of the disclosure contemplates any route of administration tolerated by the therapeutic composition. Some embodiments of the method include injection intrathecally, intracerebroventricularly, intrastriatally, intraparenchymally, or by intra-cisterna magna injection by catheterization.

Intrathecal injection is the direct injection into the spinal column or subarachnoid space. By injecting directly into the CSF of the spinal column the therapeutic oligonucleotides of the disclosure have direct access to cells (e.g., neurons and glial cells) in the spinal column and a route to access the cells in the brain by bypassing the blood brain barrier.

Intracerebroventricular (ICV) injection is a method to directly inject into the CSF of the cerebral ventricles. Similar to intrathecal injection, ICV is a method of injection which bypasses the blood brain barrier. Using ICV allows the advantage of access to the cells of the brain and spinal column without the danger of the therapeutic being degraded in the blood.

Intrastriatal injection is the direct injection into the striatum, or corpus striatum. The striatum is an area in the subcortical basal ganglia in the brain. Injecting into the striatum bypasses the blood brain barrier and the pharmacokinetic challenges of injection into the blood stream and allows for direct access to the cells of the brain.

Intraparenchymal administration is the direct injection into the parenchyma (e.g., the brain parenchyma). Injection into the brain parenchyma allows for injection directly into brain regions affected by a disease or disorder while bypassing the blood brain barrier.

Intra-cisterna magna injection by catheterization is the direct injection into the cisterna magna. The cisterna magna is the area of the brain located between the cerebellum and the dorsal surface of the medulla oblongata. Injecting into the cisterna magna results in more direct delivery to the cells of the cerebellum, brainstem, and spinal cord.

In some embodiments of the methods described herein, the therapeutic composition may be delivered to the subject by way of systemic administration, e.g., intravenously, intramuscularly, or subcutaneously.

Intravenous (IV) injection is a method to directly inject into the bloodstream of a subject. The IV administration may be in the form of a bolus dose or by way of continuous infusion, or any other method tolerated by the therapeutic composition.

Intramuscular (IM) injection is injection into a muscle of a subject, such as the deltoid muscle or gluteal muscle. IM may allow for rapid absorption of the therapeutic composition.

Subcutaneous injection is injection into subcutaneous tissue. Absorption of compositions delivered subcutaneously may be slower than IV or IM injection, which may be beneficial for compositions requiring continuous absorption.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

As used in the examples below and elsewhere throughout the present disclosure, the term “DIO” and “di-siRNA” refer to a di-branched siRNA molecule, as that term is defined herein. As used in the Examples below, “Gene A,” “Gene B,” “Gene C,” and “Gene D” all refer to different gene targets.

### Example 1. Mitigating Toxic Effects of Interfering RNA Delivery to the Central Nervous System

### Introduction

In many species, introduction of double-stranded RNA induces potent and specific gene silencing by way of RNA interference (RNAi). This phenomenon occurs in both plants and animals and has roles in viral defense and transposon silencing mechanisms. For example, short interfering RNAs (siRNAs), which are generally much shorter than the target gene, have been shown to be effective at gene silencing and are, therefore, useful as therapeutic agents for silencing genes to restore genetic and biochemical pathway activity from a disease state towards a normal, healthy state. However, delivery of therapeutic oligonucleotides, such as short interfering RNA (siRNA), to a subject, particularly to the subject's central nervous system, carries the risk of toxic side effects, including seizures, tremors, and hyperactive motor behaviors, among others. There remains a need for therapeutic oligonucleotides that effectuate reduced toxicity upon administration to a subject in need thereof.

### Methods

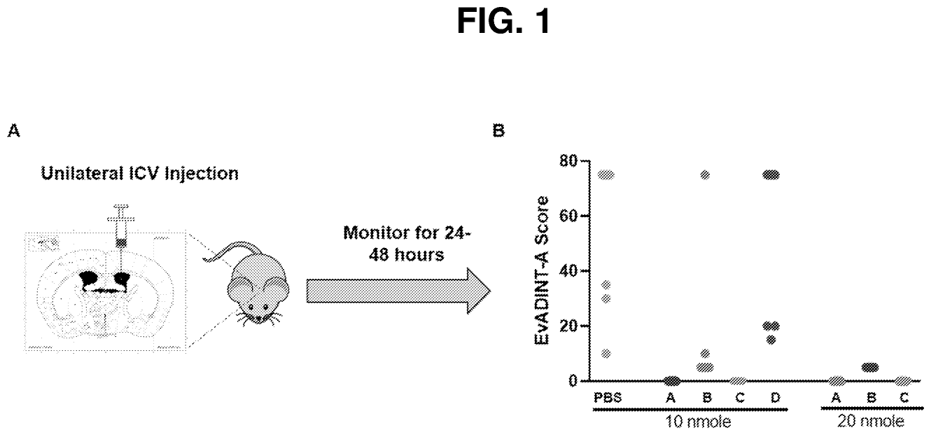

Duplex siRNA was hybridized in the presence in one of the following four ionic conditions: A) Mg²⁺; B) Ca²⁺; C) Mg²⁺ and Ca²⁺; or D) PBS only (control). Each ionically conditioned siRNA was then injected into 8-10 FVB/NJ,F mice by intracerebroventricular (ICV) injections, at two difference dosages, 10 nmol-DIO or 20 nmol-DIO, in a final volume of 10 μl (FIG. 1A). Injections occurred at a flow rate of 0.5 μl/min. A control for ICV injections (PBS only—no siRNA) was also included. Acute toxicity (e.g., seizure, death) in all animals was monitored for the next 24-48 hours. The severity of acute CNS toxicity was quantified by using an EvADINT Scoring Assay (Table 2). The higher the score, the more toxic the experimental condition was considered.

TABLE 2
EvADINT Scoring Assay

| Behavioral | Atalanta_EvADINT | | | |
|---|---|---|---|---|
| Element | Scoring Assay | | | |
| Death | 75 | | | If no major/severe acute |
| Severity | Mild | Moderate | Severe | tox effects are observed |
| Seizure/Tremor | 10 | 15 | 20 | in the first 2 hours, |
| Hyperactivity | 5 | 10 | 15 | monitoring of recovery |
| or other motor | | | | behaviors can be stopped |
| behaviors | | | | at this point. Otherwise, |
| | | | | monitoring animals for |
| | | | | behaviors described |
| | | | | below |
| Time required for | 0.5 h | 1 h | 2 h | 24 h/no recovery |
| recovery (h) | | | | |
| Sternal posture | 0 | 5 | 10 | 20 |
| Unstimulated | 0 | 5 | 10 | 15 |
| movement | | | | |
| Movement | | | | |
| without | | | | |
| ataxia | | | | |

### Results

ICV injections of siRNA hybridized in the presence of Mg2⁺ (condition A) showed no acute toxicity in mice injected with 10 or 20 nmol-DIO, resulting in 100% survival each (FIG. 1B & Table 3). ICV injections of siRNA hybridized in the presence of Ca2⁺ (condition B) showed some level of toxicity in mice injected with 10 and 20 nmol-DIO, resulting in 90 and 100% survival, respectively (FIG. 1B & Table 3). ICV injections of siRNA hybridized in the presence of both Mg2+ and Ca2+(condition C) showed no acute toxicity in mice injected with 10 and 20 nmol-DIO, resulting in 100% survival each (FIG. 1B & Table 3). ICV injections of siRNA hybridized without the presence of any divalent cations (condition D), or PBS only (no siRNA), showed much more dramatic levels of acute toxicity, resulting in 30% survival each (FIG. 1B & Table 3).

TABLE 3
Results of siRNA Ionic Conditioning Following ICV Injection

| Ionic | | | | | |
|---|---|---|---|---|---|
| conditioning | Injection | Dose | Total # | # of | Survival |
| Groups | volume (μl) | (nmol-DIO) | of animals | death | rate |
| PBS | 5 | 10 | 10 | 7 | 30% |
| A: di-siRNA | 10 | 10 | 10 | 0 | 100% |
| with Mg2⁺ | | | | | |
| B: di-siRNA | 10 | 10 | 10 | 1 | 90% |
| with Ca2⁺ | | | | | |
| C: di-siRNA | 10 | 10 | 10 | 0 | 100% |
| with Mg2⁺ | | | | | |
| & Ca2⁺ | | | | | |
| D: di-siRNA | 10 | 10 | 10 | 7 | 30% |
| in PBS | | | | | |
| A: di-siRNA | 10 | 20 | 8 | 0 | 100% |
| with Mg2⁺ | | | | | |
| B: di-siRNA | 10 | 20 | 8 | 0 | 100% |
| with Ca2⁺ | | | | | |
| C: di-siRNA | 10 | 20 | 8 | 0 | 100% |
| with Mg2⁺ | | | | | |
| & Ca2⁺ | | | | | |

### CONCLUSIONS

Delivery of therapeutic oligonucleotides to the CNS is currently challenged by acute and deadly toxic effects; however, delivery of therapeutic oligonucleotides with divalent cations significantly reduces the acute CNS toxicity.

### Example 2. Ionic Conditioning of Therapeutic Oligonucleotides does not Compromise Activity

### Gene Silencing

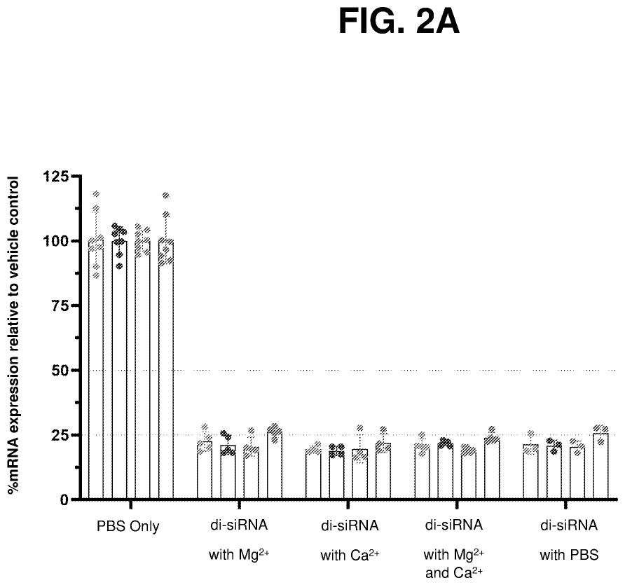

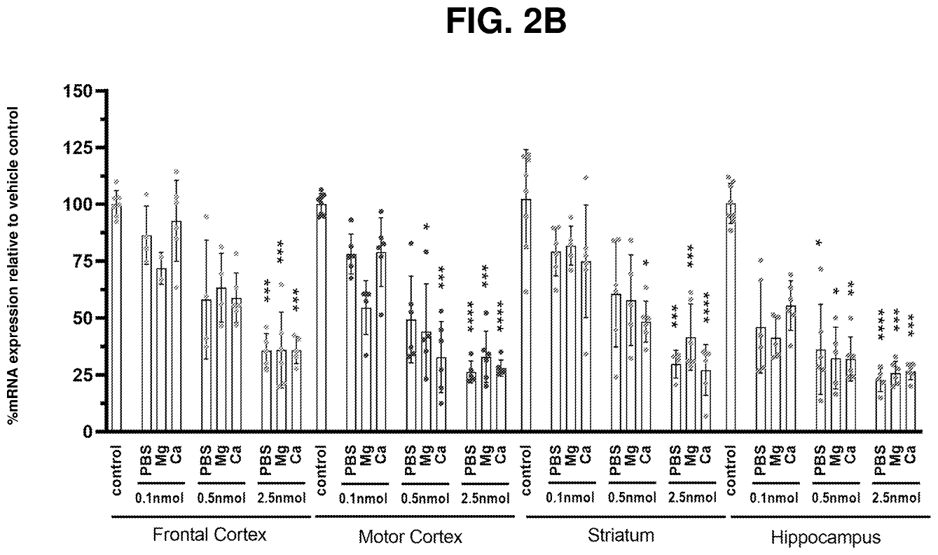

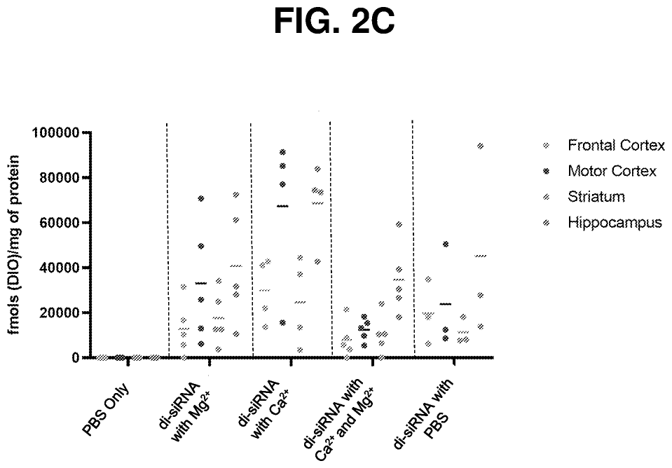

The siRNA molecules described in Example 1 were evaluated for their ability to silence a gene of interest (gene A) relative to a control. Mice were treated with a 10 nmol dose of di-siRNA and evaluated after 3 weeks for knockdown of a target gene. FIG. 2A demonstrates that di-siRNA molecules hybridized in the presence of Mg²⁺, Ca²⁺, or both Mg²⁺ and Ca²⁺ effectuate silencing of the target gene when compared to the di-siRNA molecule in PBS without a divalent cation.

In a further example, mice were treated with varying doses (0.1, 0.5, and 2.5 nmol) of a di-siRNA molecule and evaluated after 2 weeks for their ability to silence gene A relative to a control. Expression of the target gene was tested under four conditions (an untreated control, di-siRNA with PBS, di-siRNA with Mg²⁺, and di-siRNA with Ca²⁺) in each of four brain regions (frontal cortex, motor cortex, striatum, and hippocampus). Dose dependent gene silencing was observed in all three groups treated with the di-siRNA in all brain regions analyzed. Similar silencing was observed in all three conditioning groups (di-siRNA with PBS alone, with Mg²⁺, or with Ca²*) at each dose level, suggesting no impact on activity with ionic conditioning. FIG. 2B shows the results of this experiment.

### Tissue Distribution

The siRNA molecules described in Example 1 were evaluated for their distribution in certain regions of the brain relative to a control. Mice were treated with a 10 nmol dose of di-siRNA and evaluated after 3 weeks for siRNA quantification by a PNA hybridization assay. FIG. 2C demonstrates that di-siRNA molecules hybridized in the presence of Mg²⁺, Ca²⁺, or both Mg²⁺ and Ca²⁺ are effectively taken up into the frontal cortex, motor cortex, striatum, and hippocampus when compared to the di-siRNA molecule in PBS without a divalent cation.

### Example 3. Hybridization of a Di-siRNA Molecule in the Presence of a Divalent Cation has a Pro-Survival Effect

The method of introducing a divalent cation to a di-siRNA molecule was investigated. Two conditions were tested:
i) hybridization of the siRNA molecule by heating to 95° C. for 4 minutes in the presence of 100 mM NaCl, followed by a 15-minute incubation at room temperature in the presence of either 50 mM Mg²⁺ or 50 mM Ca²⁺
ii) hybridization of the siRNA molecule by heating to 95° C. for 4 minutes in the presence of 100 mM NaCl and either a) 50 mM Mg²⁺ or b) 50 mM Ca2+
   Gene A targeting di-siRNAs were prepared under these conditions, administered to mice, and evaluated for toxicity. The results of these experiments are summarized in Table 4, below. Taken together, these results are indicative of a pro-survival effect of item ii) above (heating and hybridizing in the presence of divalent cation).

TABLE 4
Results of siRNA Ionic Conditioning Following ICV Injection

| | Timing of | | | | |
|---|---|---|---|---|---|
| Ionic | Addition of | | | | |
| conditioning | Divalent | Dose | Total # of | # of | Survival |
| Groups | Cation | (nmol-DIO) | animals | death | rate |
| A: di-siRNA | Post- | 20 | 4 | 4 | 0% |
| with Mg2⁺ | hybridization | | | | |
| B: di-siRNA | Pre- | 20 | 8 | 6 | 25% |
| with Mg2⁺ | hybridization | | | | |
| C: di-siRNA | Post- | 20 | 4 | 4 | 0% |
| with Ca2⁺ | hybridization | | | | |
| D: di-siRNA | Pre- | 20 | 4 | 3 | 25% |
| with Ca2⁺ | hybridization | | | | |

### Example 4. Pro-Survival Effect of Ionic Conditioning is Correlated to the Concentration of the Ion

### Effect of Washing Protocol

A di-siRNA molecule of the disclosure targeting Gene A was hybridized in the presence of 50 mM Mg²⁺. The di-siRNA molecule was split into three groups, each of which underwent a different washing protocol:
1) Wash in 3 kDa amicon, 2×4 mL water, 1×4 mL PBS
2) Wash in 10 kDa amicon, 2×4 mL water, 1 x4 mL PBS
3) Wash in 10 kDa amicon, 4×14 mL water, 1×14 mL PBS

The concentration of Mg was calculated in each sample, and mice were injected with each sample per the protocol described in Example 1. The sample that underwent the most vigorous washing protocol contained the lowest concentration of Mg²⁺ and, consequently, was the most toxic to the animals. Table 5 summarizes the results from this experiment. Taken together, these data indicate that the presence of Mg²⁺ is critical for the toxicity benefit and is correlated to the concentration of the ion.

TABLE 5
Results of Washing Protocol Experiment

| | | # of | | |
|---|---|---|---|---|
| Wash | Concentration | animals | # of | Survival |
| Conditions | of Mg²⁺ | injected | deaths | rate |
| 1 | 48 mM | 4 | 0 | 100% |
| 2 | 41 mM | 4 | 0 | 100% |
| 3 | 10 mM | 4 | 3 | 25% |

### Determining Effective Mg²⁺ Concentrations

A di-siRNA molecule of the disclosure targeting Gene A was examined to determine the window of effective concentrations of the ion. Mice were injected with a 20 nmol dose of the di-siRNA molecule, and the amount of Mg was varied. For each concentration of ion, the effect of the hybridization protocol (adding the ion after hybridization or hybridizing in the presence of the ion as described in Example 3) was also examined. A control group of mice treated with varying concentrations of Mg in PBS without any siRNA was also included. The conditions were evaluated using the EvADINT scoring protocol described in Table 6. The higher the score, the more toxic the experimental condition was considered.

TABLE 6
EvADINT Scoring Assy

| Behavioral | | | | |
|---|---|---|---|---|
| Element | EvADINT Scoring Assay | | | |
| Death | 75 | | | If no major/severe |
| Severity | Mild | Moderate | Severe | acute tox effects are |
| Seizure | 20 | 35 | 50 | observed in the first |
| Lethargy, | 5 | 10 | 15 | 2 hours, monitoring |
| Hyperactivity | | | | of recovery behaviors |
| or other behaviors | | | | can be stopped at this |
| | | | | point. Otherwise, |
| | | | | monitoring animals for |
| | | | | behaviors described |
| | | | | below |
| Time required for | <10 m | >20 m | >1 h | 24 h/no recovery |
| recovery (h) | | | | |
| Sternal posture | 0 | 5 | 10 | 20 |

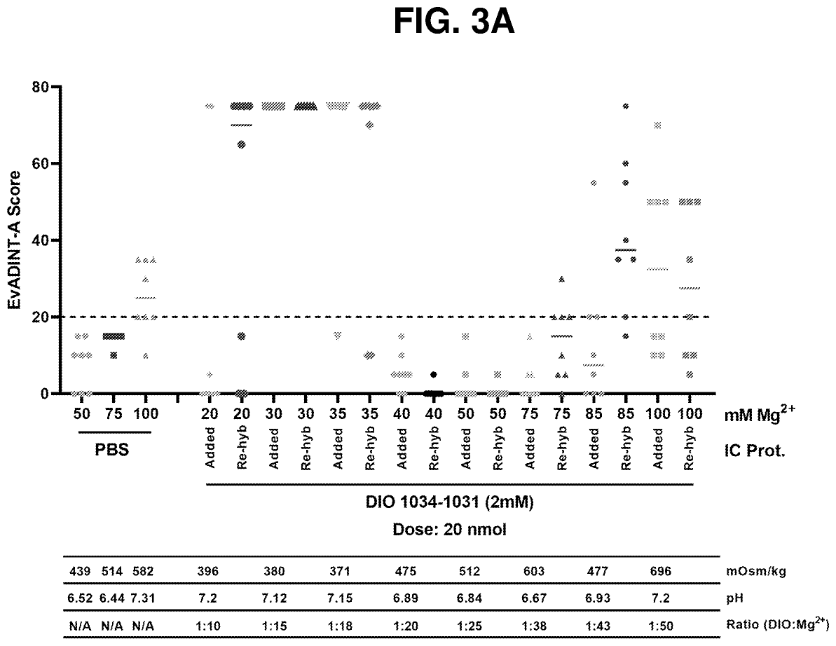

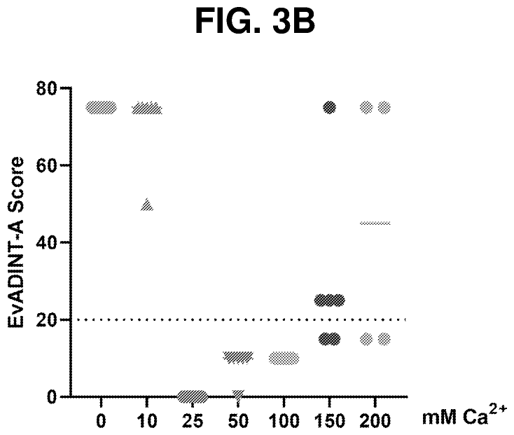

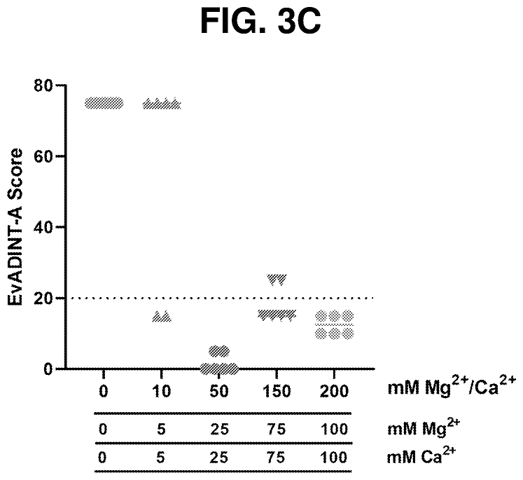

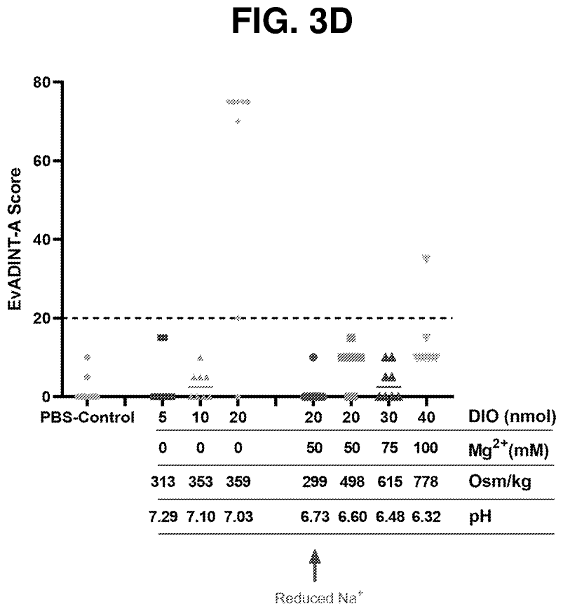

The results of these experiments are shown in FIG. 3A. These results indicate that a region of criticality may be defined, with the ideal concentration of Mg²⁺ being about 40 to about 70 mM.

Determining effective Ca²⁺ concentrations

A di-siRNA molecule of the disclosure targeting Gene A was examined to determine the window of effective concentrations of the ion. Mice were injected with a 20 nmol dose of the di-siRNA molecule, and the amount of Ca²⁺ was varied. The conditions were evaluated using the EvADINT scoring protocol described in Table 6. The higher the score, the more toxic the experimental condition was considered. The results are shown in FIG. 3B, with the window of effective concentrations falling between 25-100 mM of Ca²⁺.

Determining Effective Concentrations of Ca²⁺ and Mg²⁺ Mixtures

A di-siRNA molecule of the disclosure targeting Gene A was examined to determine the window of effective concentrations of the ion. Mice were injected with a 20 nmol dose of the di-siRNA molecule, and the amount of a 1:1 Ca²⁺/Mg²⁺ mixture was varied. The conditions were evaluated using the EvADINT scoring protocol described in Table 6. The higher the score, the more toxic the experimental condition was considered. The results are shown in FIG. 3C, with the window of effective concentrations falling between 25-100 mM of Ca²⁺

### Varying the Concentration of the Oligonucleotide and the Osmolality

Using the EvADINT-A scoring system in Table 6, the experiment was repeated while varying the concentration of the di-siRNA molecule. The molar ratio of siRNA to Mg²⁺ was held constant. In one experiment, the concentration of Na⁺ was decreased to lower the osmolality of the injection. The results of this experiment are shown in FIG. 3D. This experiment demonstrates that the siRNA molecule is not well tolerated at a dose of 20 nmol without the addition of a divalent cation. However, 20 nmol was tolerated when Mg²⁺ was added. The concentration of siRNA could be increased while still being well tolerated when the molar ratio of siRNA to Mg²⁺ was held constant. Furthermore, reducing the concentration of Na⁺ successfully reduced the osmolality without having a deleterious effect on the toxicity.

### Example 5. Ionic Conditioning Improves Tolerability of a Di-siRNA Molecule in Rats Regardless of Method of Administration

8-week-old female Sprague-Dawley rats were treated with a di-siRNA molecule of the disclosure targeting Gene A, either by unilateral intracerebroventricular direct brain injection (ICV) or intrathecal injection (IT) at a flow rate of 5 uL/min. Table 7 summarizes the results of rats treated with the siRNA molecule without a divalent cation, whereas Table 8 summarizes the results of rats treated with the siRNA molecule with a divalent cation at a ratio of 1:25. As is evident from Table 7 and Table 8, ionic conditioning greatly improves the tolerability of the siRNA molecule when Mg²⁺ is added, and the benefit is observed regardless of the method of administration.

TABLE 7
Tolerability of a di-siRNA molecule in rats without ionic conditioning

| Group | # of animals | Died | Survived | % Survival | Seized/Vocaliz. | % Seized |
|---|---|---|---|---|---|---|
| ICV bilateral, PBS | 6 | 0 | 6 | 100.0 | 0.0 | 0.0 |
| ICV bilateral, siRNA, 60 nmol | 1 | 0 | 1 | 100.0 | 1.0 | 100.0 |
| ICV bilateral, siRNA, 40 nmol | 4 | 0 | 4 | 100.0 | 2.0 | 50.0 |
| ICV bilateral, siRNA, 30 nmol | 4 | 0 | 4 | 100.0 | 4.0 | 100.0 |
| ICV bilateral, siRNA, 15 nmol | 6 | 0 | 6 | 100.0 | 0.0 | 0.0 |
| IT, PBS | 6 | 0 | 6 | 100.0 | 0.0 | 0.0 |
| IT, siRNA, 60 nmol | 6 | 2 | 4 | 66.7 | 5.0 | 83.3 |
| IT, siRNA, 40 nmol | 6 | 1 | 5 | 83.3 | 3.0 | 50.0 |
| IT, siRNA, 30 nmol | 6 | 0 | 6 | 100 | 0 | 0 |

TABLE 8
Tolerability of a di-siRNA molecule in rats with ionic conditioning

| Group | # of animals | Died | Survived | % Survival | Seized/Vocaliz. | % Seized/Vocal |
|---|---|---|---|---|---|---|
| ICV unilateral, PBS | 6 | 0 | 6 | 100 | 0 | 0 |
| (50 mM Mg2+) | | | | | | |
| ICV unilateral, siRNA, | 6 | 0 | 6 | 100 | 0 | 0 |
| 60 nmol | | | | | | |
| ICV unilateral, siRNA, | 6 | 0 | 6 | 100 | 0 | 0 |
| 120 nmol | | | | | | |
| ICV unilateral, siRNA, | 1 | 0 | 1 | 100 | 0 | 0 |
| 180 nmol | | | | | | |
| ICV unilateral, siRNA, | 2 | 0 | 2 | 100 | 0 | 0 |
| 240 nmol | | | | | | |
| IT, PBS (50 mM Mg2+) | 6 | 0 | 6 | 100 | 0 | 0 |
| IT, siRNA, 60 nmol | 6 | 0 | 6 | 100 | 0 | 0 |
| IT, siRNA, 120 nmol | 6 | 0 | 6 | 100 | 0 | 0 |
| IT, siRNA, 180 nmol | 2 | 0 | 2 | 100 | 0 | 0 |
| IT, siRNA, 240 nmol | 3 | 0 | 3 | 100 | 0 | 0 |

### Example 6. Ionic Conditioning Improves Tolerability of Antisense Oligonucleotides and Mono-siRNA Molecules

In addition to the di-siRNA molecules tested in the above examples, the effect of the addition of one or more divalent cations on the tolerability of a single stranded antisense oligonucleotide and a mono-siRNA was also investigated.

### Antisense Oligonucleotide

20 or 40 nmol of an antisense oligonucleotide of the disclosure targeting Malat-1 was administered to mice via unilateral ICV injection with varying concentrations of Mg²⁺. Antisense oligonucleotides targeting Malat-1 were previously shown to be slightly less toxic when formulated as a salt with Ca²⁺ (Moazami et al., BioRxiv. 2021). Each condition was evaluated for the number of animals exhibiting seizure and/or death. The results of this assay are shown in Table 9, below. As is evident from the results, there was a decrease in toxicity when divalent cations were added, notably when 20 nmol of ASO is administered.

TABLE 9
Tolerability of an antisense oligonucleotide in mice

| | # of | | % | | % |
|---|---|---|---|---|---|
| ASO | animals | Survived | Survival | Seized | Seized |
| ASO (40 nmol), | 8 | 4 | 50 | 8 | 100 |
| no Mg²⁺ | | | | | |
| ASO (40 nmol), | 8 | 1 | 13 | 0 | 0 |
| Mg²⁺ 25 mM | | | | | |
| ASO (40 nmol), | 8 | 0 | 0 | 3 | 38 |
| Mg²⁺12.5 mM | | | | | |
| ASO (20 nmol), | 6 | 2 | 33 | 6 | 100 |
| no Mg²⁺ | | | | | |
| ASO (20 nmol), | 6 | 6 | 100 | 2 | 33 |
| Mg²⁺ 12.5 mM | | | | | |

### Mono-siRNA

40 nmol of a mono-siRNA of the disclosure targeting Gene A was administered to mice via unilateral ICV injection with varying concentrations of Mg²⁺. Each condition was evaluated for the number of animals exhibiting seizure and/or death. The results of this assay are shown in Table 10, below. As is evident from the results, there was a marked decrease in toxicity when divalent cations were added.

TABLE 10
Tolerability of a mono-siRNA in mice

| | # of | | % | | % |
|---|---|---|---|---|---|
| Mono-siRNA | animals | Survived | Survival | Seized | Seized |
| Mono siRNA | 8 | 0 | 0 | 8 | 100 |
| (40 nmol), | | | | | |
| no Mg²⁺ | | | | | |
| Mono siRNA | 8 | 8 | 100 | 0 | 0 |
| (40 nmol), | | | | | |
| 50 mM Mg²⁺ | | | | | |

### Example 7. Ionic Conditioning Improves Tolerability of Oligonucleotides Regardless of Sequence or Gene Target

Three di-siRNA molecules having different sequences and targeting different genes were tested for their toxicity under various conditions with and without divalent cations

### Di-siRNA B and Di-siRNA C

20 nmol of two separate di-siRNA molecules of the disclosure, di-siRNA B (targeting Gene B) and di-siRNA C (targeting Gene C), were administered to mice via unilateral ICV injection with varying concentrations of Mg²⁺. Each of di-siRNA A and di-siRNA B have a different nucleobase sequence and target a different gene from each other and from di-siRNA molecules mentioned in any foregoing example. Each condition tested was evaluated for the number of animals exhibiting seizure and/or death. The results of this assay are shown in Table 11, below. These results demonstrate that the addition of a divalent cation to a therapeutic oligonucleotide has a toxicity benefit regardless of nucleobase sequence or target gene.

TABLE 11
Tolerability of di-siRNA molecules
with differing sequences in mice

| | # of | | % | | % |
|---|---|---|---|---|---|
| di-siRNA | animals | Survived | Survival | Seized | Seized |
| di-siRNA B (20 nmol), | 8 | 8 | 100 | 8 | 100 |
| 50 mM Mg²⁺ | | | | | |
| di-siRNA B (20 nmol), | 8 | 0 | 0 | 8 | 100 |
| no Mg²⁺ | | | | | |
| di-siRNA C (20 nmol), | 8 | 8 | 100 | 4 | 50 |
| 50 mM Mg²⁺ | | | | | |
| di-siRNA C (20 nmol), | 8 | 4 | 50 | 7 | 87.5 |
| no Mg²⁺ | | | | | |

di-siRNA D

Varying doses of a di-siRNA molecules of the disclosure, di-siRNA 0 (targeting Gene 0), was administered to mice with varying concentrations of Mg²⁺. di-siRNA 0 has a different nucleobase sequence and targets a different gene from di-siRNA B, di-siRNA C, or any of the di-siRNA molecules mentioned in any foregoing example. Each condition tested was evaluated for the number of animals exhibiting seizure and/or death. The results of this assay are shown in Table 12, below. These results demonstrate that the addition of a divalent cation to a therapeutic oligonucleotide has a toxicity benefit regardless of nucleobase sequence or target gene.

TABLE 12
Tolerability of di-siRNA C in mice

| | # of | | % | | % |
|---|---|---|---|---|---|
| | animals | Survived | Survival | seized | seized |
| di-siRNA C | 6 | 6 | 100 | 1 | 17 |
| (20 nmol), no Mg²⁺ | | | | | |
| di-siRNA C | 6 | 6 | 100 | 0 | 0 |
| (10 nmol), no Mg²⁺ | | | | | |
| di-siRNA C 40 nmol; | 6 | 6 | 100 | 5 | 83 |
| 100 mM Mg²⁺ | | | | | |
| di-siRNA C, 30 nmol; | 6 | 6 | 100 | 0 | 0 |
| 75 mM Mg²⁺ | | | | | |
| di-siRNA C, 20 nmol; | 6 | 6 | 100 | 0 | 0 |
| 50 mM Mg²⁺ | | | | | |

### Example 8. Method of Treating a Patient in Need of Gene Silencing

A subject in need of gene silencing in the cells of their central nervous system is treated with a dosage of a therapeutic oligonucleotide, formulated as a salt, at frequency determined by a practitioner. For example, a physician could start prescribing doses of one of the therapeutic oligonucleotides of the disclosure (e.g., siRNA) at levels lower than that required to achieve the desired therapeutic effect and gradually increase the dosage until the desired effect is achieved. Alternatively, a physician may begin a treatment regimen by administering one of the therapeutic oligonucleotides of the disclosure at a high dose and subsequently administer progressively lower doses until a therapeutic effect is achieved (e.g., a reduction in expression of a target gene sequence). In general, a suitable daily dose of one of one of the therapeutic oligonucleotides of the disclosure (e.g., siRNA) will be an amount which is the lowest dose effective to produce a therapeutic effect. The ss- or ds-therapeutic oligonucleotides of the disclosure may be administered by injection, e.g., intrathecally, intracerebroventricularly, intrastriatally or by intra-cisterna magna injection via catheterization (e.g., injection into the caudate nucleus or putamen). A daily dose of a therapeutic composition of one of the therapeutic oligonucleotides of the disclosure may be administered as a single dose or as two, three, four, five, six or more doses administered separately at appropriate intervals throughout the day, week, month, or year, optionally, in unit dosage forms. While it is possible for any of the therapeutic oligonucleotides of the disclosure to be administered alone, it may also be administered as a pharmaceutical formulation in combination with excipients, carriers, and optionally, additional therapeutic agents. Dosage and frequency are determined based on the subject's height, weight, age, sex, and other disorders.

The therapeutic oligonucleotide is selected by the practitioner for compatibility with the disease and subject. Single- or double-stranded therapeutic oligonucleotides (e.g., branched siRNA) are available for selection. The therapeutic oligonucleotide chosen has an antisense strand and may have a sense strand with a sequence and RNA modifications (e.g., natural and non-natural internucleoside linkages, modified sugars, 5′-phosphorus stabilizing moieties, and ionically bonded divalent cations) best suited to the patient and the disease being targeted.

The therapeutic oligonucleotide is delivered by the route best suited the patient (e.g., intrathecally, intracerebroventricularly, intrastriatally or by intra-cisterna magna injection via catheterization) and condition at a rate tolerable to the patient until the subject has reached a maximum tolerated dose, or until the symptoms of the disease are ameliorated satisfactorily.

### SPECIFIC EMBODIMENTS

Exemplary embodiments of the invention are listed below. The below enumerated embodiments should not be construed to limit the scope of the disclosure; rather, the below are presented as examples of the utility of the disclosure.

E1. A method of delivering a therapeutic oligonucleotide (e.g., siRNA, ASO, miRNA, gRNA, etc.) to a subject, the method comprising administering the therapeutic oligonucleotide in the form of a salt comprising one or more divalent cations, optionally wherein the therapeutic oligonucleotide is an interfering RNA molecule (e.g., siRNA, shRNA, or miRNA).

E2. The method of any one of E1 wherein the therapeutic oligonucleotide comprises a plurality of cationic binding sites that are partially or fully saturated by the one or more divalent cations.

E3. The method of any one of E1-E2, wherein the degree of saturation of the cationic binding sites by the one or more divalent cations is from about 10% to about 100%.

E4. The method of E3, wherein the degree of saturation of the cationic binding sites by the one or more divalent cations is from about 20% to about 100%.

E5. The method of E4, wherein the degree of saturation of the cationic binding sites by the one or more divalent cations is from about 30% to about 100%.

E6. The method of E5, wherein the degree of saturation of the cationic binding sites by the one or more divalent cations is from about 40% to about 100%.

E7. The method of E6, wherein the degree of saturation of the cationic binding sites by the one or more divalent cations is from about 50% to about 100%.

E8. The method of E7, wherein the degree of saturation of the cationic binding sites by the one or more divalent cations is from about 60% to about 100%.

E9. The method of E8, wherein the degree of saturation of the cationic binding sites by the one or more divalent cations is from about 70% to about 100%.

E10. The method of E9, wherein the degree of saturation of the cationic binding sites by the one or more divalent cations is from about 80% to about 100%.

E11. The method of E10, wherein the degree of saturation of the cationic binding sites by the one or more divalent cations is from about 90% to about 100%.

E12. The method of any one of E1-E11, wherein the cationic binding site is located within an internucleoside linkage, optionally wherein the internucleoside linkage is selected from a phosphodiester linkage and a phosphorothioate linkage.

E13. The method of any one of E1-E12, wherein the one or more divalent cations is characterized by an ionic radius of from about 30 picometers to about 150 picometers.

E14. The method of E13, wherein the one or more divalent cations is characterized by an ionic radius of from about 30 picometers to about 140 picometers.

E15. The method of E14, wherein the one or more divalent cations is characterized by an ionic radius of from about 40 picometers to about 130 picometers.

E16. The method of E15, wherein the one or more divalent cations is characterized by an ionic radius of from about 50 picometers to about 120 picometers.

E17. The method of E16, wherein the one or more divalent cations is characterized by an ionic radius of from about 60 picometers to about 110 picometers.

E18. The method of any one of E1-E12, wherein the one or more divalent cations is characterized by an ionic radius of from about 60 picometers to about 100 picometers.

E19. The method E18, wherein the one or more divalent cations is characterized by an ionic radius of from about 60 picometers to about 90 picometers.

E20. The method of any one of E1-E12, wherein the one or more divalent cations comprise Ba²⁺, Be²⁺, Ca²⁺, Cu²⁺, Mg²⁺, Mn²⁺, Ni²⁺, or Zn²⁺, or a combination thereof.

E21. The method of E20, wherein the one or more divalent cations comprise Ba²⁺.

E22. The method of E20 or E21, wherein the one or more divalent cations comprise Be²⁺.

E23. The method of any one of E20-E22, wherein the one or more divalent cations comprise Ca²⁺.

E24. The method of any one of E20-E23, wherein the one or more divalent cations comprise Cu²⁺.

E25. The method of any one of E20-E24, wherein the one or more divalent cations comprise Mg²⁺.

E26. The method of any one of E20-E25, wherein the one or more divalent cations comprise Mn²⁺.

E27. The method of any one of E20-E26, wherein the one or more divalent cations comprise Ni²⁺.

E28. The method of any one of E20-E27, wherein the one or more divalent cations comprise Zn²⁺.

E29. The method of any one of E20-E28, wherein the one or more divalent cations comprise Ca²⁺ and Mg²⁺.

E30. The method of E29, wherein the Ca²⁺ and Mg²⁺ are present in a 1:1 ratio.

E31. The method of any one of E1-E30, wherein the one or more divalent cations comprise a hard Lewis acid.

E32. The method of any one of E1-E31, wherein the one or more divalent cations displaces water from a cationic binding site of the therapeutic oligonucleotide.

E33. The method of any one of E1-E32, wherein the therapeutic oligonucleotide is a short interfering RNA (siRNA), a short hairpin RNA (shRNA), a microRNA (miRNA), a CRISPR guide RNA (gRNA), or an RNA antisense oligonucleotide (ASO).

E34. The method of any one of E1-E33, wherein the therapeutic oligonucleotide is a short interfering RNA (siRNA) molecule.

E35. The method of any one of E1-E33, wherein the therapeutic oligonucleotide is an antisense oligonucleotide (ASO).

E36. The method of E35, wherein the siRNA molecule is branched, optionally wherein the siRNA molecule is di-branched, tri-branched, or tetra-branched.

E37. The method of E36, wherein the siRNA molecule is di-branched.

E38. The method of E36, wherein the siRNA molecule is tri-branched.

E39. The method of E36, wherein the siRNA molecule is tetra-branched.

E40. The method of E36 or E37, wherein the di-branched siRNA molecule is represented by any one of Formulas I-III:

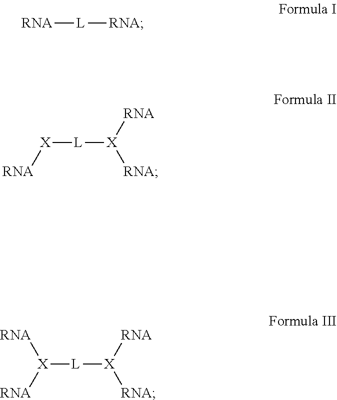

wherein each RNA is, independently, an siRNA molecule, L is a linker, and each X, independently, represents a branch point moiety.

E41. The method of E40, wherein the di-branched siRNA molecule is represented by Formula I.

E42. The method of E40, wherein the di-branched siRNA molecule is represented by Formula II.

E43. The method of E40, wherein the di-branched siRNA molecule is represented by Formula III.

E44. The method of E36 or E38, wherein the tri-branched siRNA molecule is represented by any one of Formulas IV-VII:

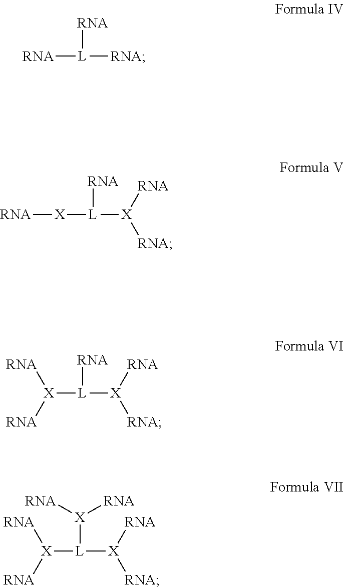

wherein each RNA is, independently, an siRNA molecule, L is a linker, and each X, independently, represents a branch point moiety.

E45. The method of E44, wherein the tri-branched siRNA molecule is represented by Formula IV.

E46. The method of E44, wherein the tri-branched siRNA molecule is represented by Formula V.

E47. The method of E44, wherein the tri-branched siRNA molecule is represented by Formula VI.

E48. The method of E44, wherein the tri-branched siRNA molecule is represented by Formula VII.

E49. The method of E36 or E39, wherein the tetra-branched siRNA molecule is represented by any one of Formulas VIII-XII:

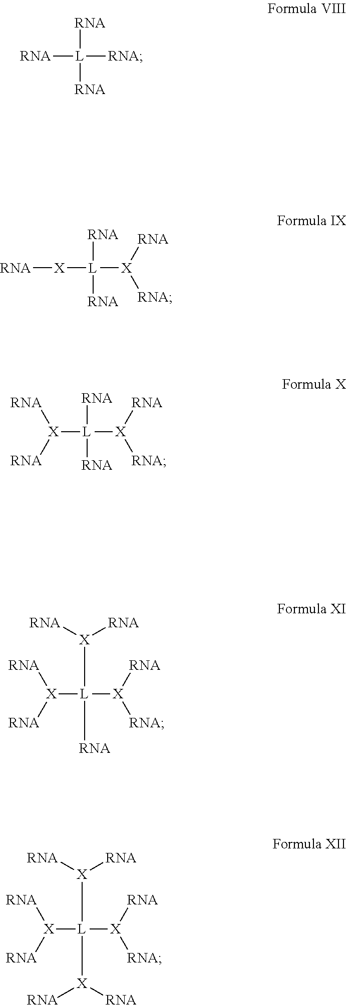

wherein each RNA is, independently, an siRNA molecule, L is a linker, and each X, independently, represents a branch point moiety.

E50. The method of E49, wherein the tetra-branched siRNA molecule is represented by Formula VIII.

E51. The method of E49, wherein the tetra-branched siRNA molecule is represented by Formula IX.

E52. The method of E49, wherein the tetra-branched siRNA molecule is represented by Formula X.

E53. The method of E49, wherein the tetra-branched siRNA molecule is represented by Formula XI.

E54. The method of E49, wherein the tetra-branched siRNA molecule is represented by Formula XII.

E55. The method of any one of E40-E54, wherein the linker is selected from a group consisting of one or more contiguous subunits of an ethylene glycol (e.g., polyethylene glycol (PEG), such as, e.g., triethylene glycol (TrEG) or tetraethylene glycol (TEG)), alkyl, carbohydrate, block copolymer, peptide, RNA, and DNA.

E56. The method of E55, wherein the linker is an ethylene glycol oligomer.

E57. The method of E56, wherein the ethylene glycol oligomer is a PEG.

E58. The method of E57, wherein the PEG a TrEG.

E59. The method of E57, wherein the PEG is a TEG.

E60. The method of E55, wherein the linker is an alkyl oligomer.

E61. The method of E55, wherein the linker is a carbohydrate oligomer.

E62. The method of E55, wherein the linker is a block copolymer.

E63. The method of E55, wherein the linker is a peptide oligomer.

E64. The method of E55, wherein the linker is an RNA oligomer.

E65. The method of E55, wherein the linker is a DNA oligomer.

E66. The method of any one of E55-E65, wherein the oligomer or copolymer contains 2 to 20 contiguous subunits.

E67. The method of E66, wherein oligomer or copolymer contains 4 to 18 contiguous subunits.

E68. The method of E67, wherein oligomer or copolymer contains 6 to 16 contiguous subunits.

E69. The method of E68, wherein oligomer or copolymer contains 8 to 14 contiguous subunits.

E70. The method of E69, wherein oligomer or copolymer contains 10 to 12 contiguous subunits.

E71. The method of E55, wherein the linker attaches one or more (e.g., 1, 2, or more) siRNA molecules by way of a covalent bond-forming moiety, optionally wherein the covalent bond-forming moiety is selected from the group consisting of an alkyl, ester, amide, carbamate, phosphonate, phosphate, phosphorothioate, phosphoroamidate, triazole, urea, and formacetal.

E72. The method of E55, wherein the linker includes a structure of Formula L1:

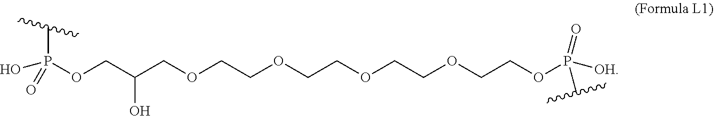 [SMILES: *P(=O)(O)OCCOCCOCCOCCOCC(O)COP(*)(=O)O]

E73. The method of E55, wherein the linker includes a structure of Formula L2:

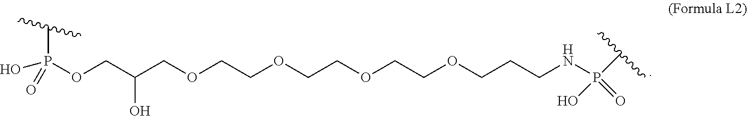 [SMILES: *P(=O)(O)NCCCOCCOCCOCCOCC(O)COP(*)(=O)O]

E74. The method of E55, wherein the linker includes a structure of Formula L3:

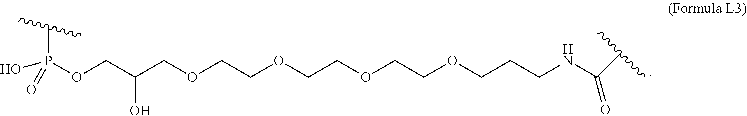 [SMILES: *C(=O)NCCCOCCOCCOCCOCC(O)COP(*)(=O)O]

E75. The method of E55, wherein the linker includes a structure of Formula L4:

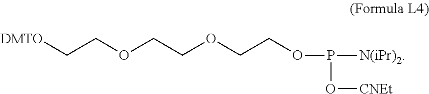

E76. The method of E55, wherein the linker includes a structure of Formula L5:

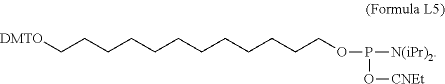

E77. The method of E55, wherein the linker includes a structure of Formula L6:

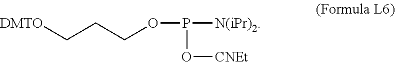

E78. The method of E55, wherein the linker includes a structure of Formula L7:

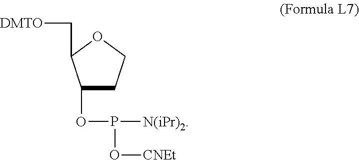

E79. The method of E55, wherein the linker includes a structure of Formula L8:

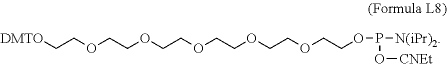

E80. The method of E55, wherein the linker includes a structure of Formula L9:

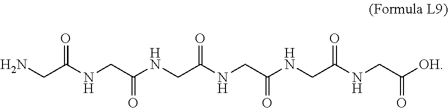 [SMILES: NCC(=O)NCC(=O)NCC(=O)NCC(=O)NCC(=O)NCC(=O)O]

E81. The method of any one of E1-E80, wherein
a. the therapeutic oligonucleotide comprises an antisense strand and a sense strand having complementarity to the antisense strand; or
b. the therapeutic oligonucleotide is an antisense oligonucleotide comprising an antisense strand only.

E82. The method of E81, wherein the antisense strand and sense strand comprises alternating 2′-O-methyl and 2′-fluoro ribonucleosides.

E83. The method of E81 or E82, wherein the antisense strand has the following formula, in the 5′-to-3′ direction:

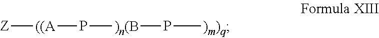

wherein Z is a 5′ phosphorus stabilizing moiety;
each A is, independently, a 2′-O-methyl (2′-O-Me) ribonucleoside;
each B is, independently, a 2′-fluoro (2′-F) ribonucleoside;
each P is, independently, an internucleoside linkage selected from a phosphodiester linkage and a phosphorothioate linkage;
n is an integer from 1 to 5;
m is an integer from 1 to 5; and
q is an integer between 1 and 30.

E84. The method of E83, wherein n is from 1 to 4.

E85. The method of E83, wherein n is from 1 to 3.

E86. The method of E83, wherein n is from 1 to 2.

E87. The method of E83, wherein n is 1.

E88. The method of E83, wherein n is 2.

E89. The method of E83, wherein n is 3.

E90. The method of E83, wherein n is 4.

E91. The method of E83, wherein n is 5.

E92. The method of any one of E83-E91, wherein m is from 1 to 4.

E93. The method of E92, wherein m is from 1 to 3.

E94. The method of E92, wherein m is from 1 to 2.

E95. The method of E92, wherein m is 1.

E96. The method of E92, wherein m is 2.

E97. The method of E92, wherein m is 3.

E98. The method of E92, wherein m is 4.

E99. The method of E92, wherein m is 5.

E100. The method of E81, wherein the antisense strand comprises a structure represented by Formula I, wherein Formula I is, in the 5′-to-3′ direction:

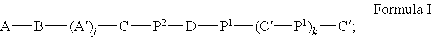

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

E101. The method of E100, wherein the antisense strand comprises a structure represented by Formula A1, wherein Formula A1 is, in the 5′-to-3′ direction:

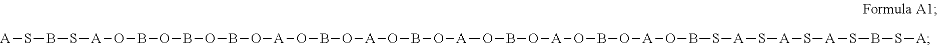

wherein A represents a 2′-O-Me ribonucleoside, B represents a 2′-F ribonucleoside, O represents a phosphodiester internucleoside linkage, and S represents a phosphorothioate internucleoside linkage.

E102. The method of E100, wherein the antisense strand comprises a structure represented by Formula II, wherein Formula II is, in the 5′-to-3′ direction:

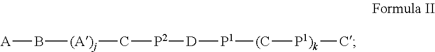

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

E103. The method of E102, wherein the antisense strand comprises a structure represented by Formula A2, wherein Formula A2 is, in the 5′-to-3′ direction:

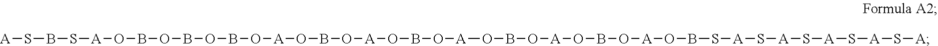

wherein A represents a 2′-O-Me ribonucleoside, B represents a 2′-F ribonucleoside, O represents a phosphodiester internucleoside linkage, and S represents a phosphorothioate internucleoside linkage.

E104. The method of any one of E81-E103, wherein the sense strand comprises a structure represented by Formula III, wherein Formula III is, in the 5′-to-3′ direction:

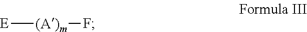

wherein E is represented by the formula (C—P¹)₂;
F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D;
A′, C, D, P¹, and P² are as defined in Formula II; and
m is an integer from 1 to 7.

E105. The method of E104, wherein the sense strand comprises a structure represented by Formula S1, wherein Formula S1 is, in the 5′-to-3′ direction:

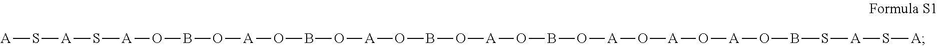

wherein A represents a 2′-O-Me ribonucleoside, B represents a 2′-F ribonucleoside, O represents a phosphodiester internucleoside linkage, and S represents a phosphorothioate internucleoside linkage.

E106. The method of E104, wherein the sense strand comprises a structure represented by Formula S2, wherein Formula S2 is, in the 5′-to-3′ direction:

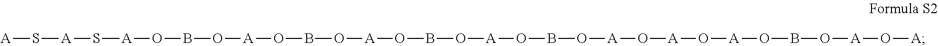

wherein A represents a 2′-O-Me ribonucleoside, B represents a 2′-F ribonucleoside, O represents a phosphodiester internucleoside linkage, and S represents a phosphorothioate internucleoside linkage.

E107. The method of E104, wherein the sense strand comprises a structure represented by Formula S3, wherein Formula S3 is, in the 5′-to-3′ direction:

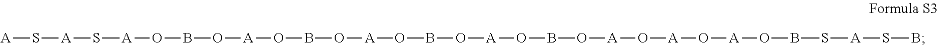

wherein A represents a 2′-O-Me ribonucleoside, B represents a 2′-F ribonucleoside, O represents a phosphodiester internucleoside linkage, and S represents a phosphorothioate internucleoside linkage.

E108. The method of E104, wherein the sense strand comprises a structure represented by Formula S4, wherein Formula S4 is, in the 5′-to-3′ direction:

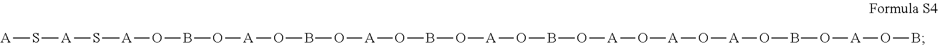

wherein A represents a 2′-O-Me ribonucleoside, B represents a 2′-F ribonucleoside, O represents a phosphodiester internucleoside linkage, and S represents a phosphorothioate internucleoside linkage.

E109. The method of any one of E81, E82, and E104-E108, wherein the antisense strand comprises a structure represented by Formula IV, wherein Formula IV is, in the 5′-to-3′ direction:

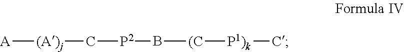

wherein A is represented by the formula C—P¹-D-P;
each A′ is represented by the formula C—P²-D-P²;
B is represented by the formula D-P¹—C—P²-D-P²;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

E110. The method of E109, wherein the antisense strand comprises a structure represented by Formula A3, wherein Formula A3 is, in the 5′-to-3′ direction

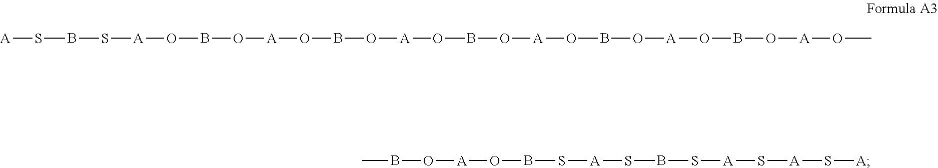

wherein A represents a 2′-O-Me ribonucleoside, B represents a 2′-F ribonucleoside, O represents a phosphodiester internucleoside linkage, and S represents a phosphorothioate internucleoside linkage.

E111. The method of any one of E81-E103, E109, and E110, wherein the sense strand comprises a structure represented by Formula V, wherein Formula V is, in the 5′-to-3′ direction:

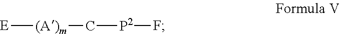

wherein E is represented by the formula (C—P¹)₂;
F is represented by the formula D-P¹—C—P¹—C, D-P²—C—P²—C, D-P¹—C—P¹-D, or D-P²—C—P²-D;
A′, C, D, P¹ and P² are as defined in Formula IV; and
m is an integer from 1 to 7.

E112. The method of E111, wherein the sense strand comprises a structure represented by Formula S5, wherein Formula S5 is, in the 5′-to-3′ direction:

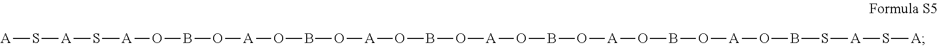

wherein A represents a 2′-O-Me ribonucleoside, B represents a 2′-F ribonucleoside, O represents a phosphodiester internucleoside linkage, and S represents a phosphorothioate internucleoside linkage.

E113. The method of E111, wherein the sense strand comprises a structure represented by Formula S6, wherein Formula S6 is, in the 5′-to-3′ direction:

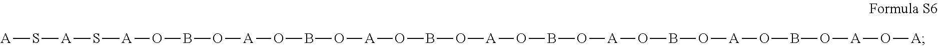

wherein A represents a 2′-O-Me ribonucleoside, B represents a 2′-F ribonucleoside, O represents a phosphodiester internucleoside linkage, and S represents a phosphorothioate internucleoside linkage

E114. The method of E111, wherein the sense strand comprises a structure represented by Formula S7, wherein Formula S7 is, in the 5′-to-3′ direction:

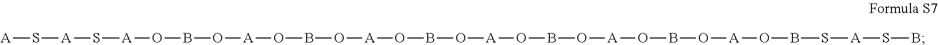

wherein A represents a 2′-O-Me ribonucleoside, B represents a 2′-F ribonucleoside, O represents a phosphodiester internucleoside linkage, and S represents a phosphorothioate internucleoside linkage.

E115. The method of E111, wherein the sense strand comprises a structure represented by Formula S8, wherein Formula S8 is, in the 5′-to-3′ direction:

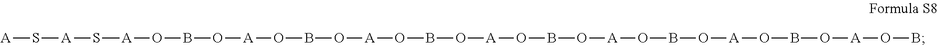

wherein A represents a 2′-O-Me ribonucleoside, B represents a 2′-F ribonucleoside, O represents a phosphodiester internucleoside linkage, and S represents a phosphorothioate internucleoside linkage.

E116. The method of any one of E81, E82, E104-E108, and E111-E114, wherein the antisense strand comprises a structure represented by Formula VI, wherein Formula VI is, in the 5′-to-3′ direction:

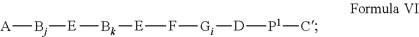

wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7;
k is an integer from 1 to 7; and
l is an integer from 1 to 7.

E117. The method of E116, wherein the antisense strand comprises a structure represented by Formula A4, wherein Formula A4 is, in the 5′-to-3′ direction:

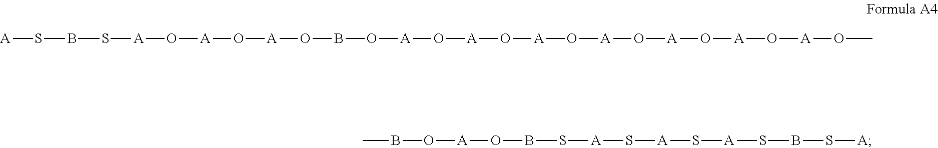

wherein A represents a 2′-O-Me ribonucleoside, B represents a 2′-F ribonucleoside, O represents a phosphodiester internucleoside linkage, and S represents a phosphorothioate internucleoside linkage.

E118. The method of any one of E81-E103, E109, E110, E116, and E117, wherein the sense strand comprises a structure represented by Formula VII, wherein Formula VII is, in the 5′-to-3′ direction:

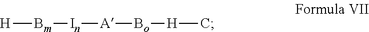

wherein A′ is represented by the formula C—P²-D-P²;
each H is represented by the formula (C—P¹)₂;
each I is represented by the formula (D-P²);
B, C, D, P¹ and P² are as defined in Formula VI;
m is an integer from 1 to 7;
n is an integer from 1 to 7; and
o is an integer from 1 to 7.

E119. The method of E118, wherein the sense strand comprises a structure represented by Formula s9, wherein Formula S9 is, in the 5′-to-3′ direction:

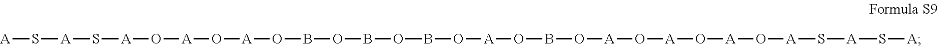

wherein A represents a 2′-O-Me ribonucleoside, B represents a 2′-F ribonucleoside, O represents a phosphodiester internucleoside linkage, and S represents a phosphorothioate internucleoside linkage.

E120. The method of any one of E81-E119, wherein the antisense strand further comprises a 5′-phosphorus stabilizing moiety at the 5′ end of the antisense strand.

E121. The method of any one of E81-E120, wherein the sense strand further comprises a 5′-phosphorus stabilizing moiety at the 5′ end of the sense strand.

E122. The method of any one of E81-E97, E120, and E121, wherein the 5′-phosphorus stabilizing moiety is represented by any one of Formulas IX-XVI:

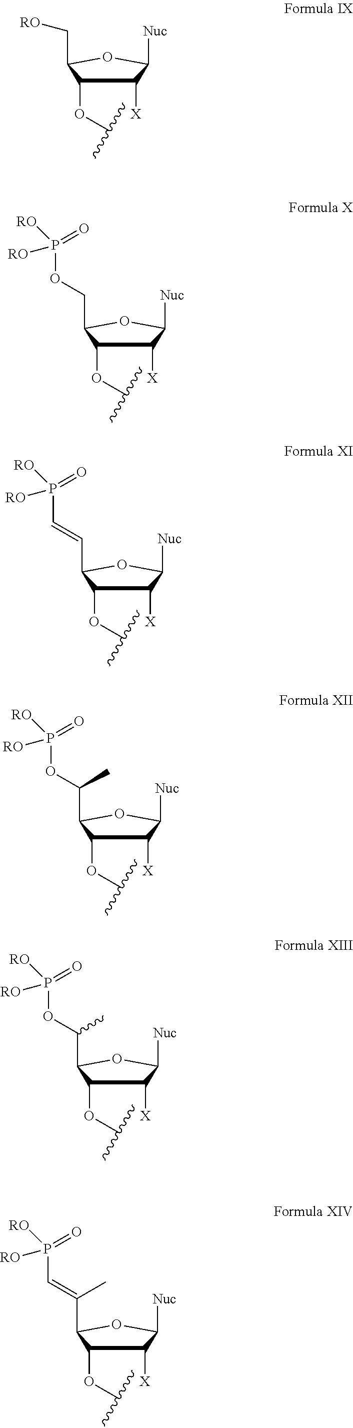

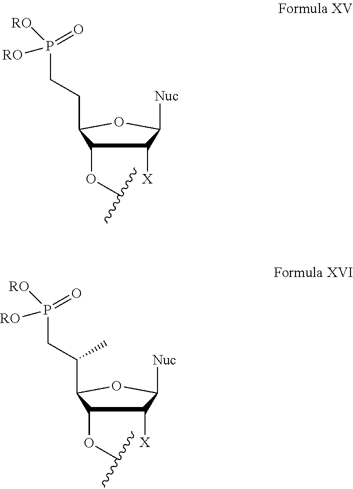

wherein Nuc represents a nucleobase selected from the group consisting of adenine, uracil, guanine, thymine, and cytosine, and R represents optionally substituted alkyl, optionally substituted alkenyl, optionally substituted alkynyl, phenyl, benzyl, hydroxy, or hydrogen.

E123. The method of E122, wherein the 5′-phosphorus stabilizing moiety is (E)-vinylphosphonate represented in Formula XVI.

E124. The method of any one of E82-E123, wherein at least 50% of the ribonucleosides are 2′-O-Me ribonucleoside.

E125. The method of any one of E82-E124, wherein at least 60% of the ribonucleosides are 2′-O-Me ribonucleoside.

E126. The method of any one of E82-E125, wherein at least 70% of the ribonucleosides are 2′-O-Me ribonucleoside.

E127. The method of any one of E82-E126, wherein at least 80% of the ribonucleosides are 2′-O-Me ribonucleoside.

E128. The method of any one of E82-E127, wherein at least 90% of the ribonucleosides are 2′-O-Me ribonucleoside.

E129. The method of any one of E81-E128, wherein the length of the antisense strand is between 10 and 30 nucleotides.

E130. The method of any one of E81-E129, wherein the length of the antisense strand is between 15 and 25 nucleotides.

E131. The method of E130, wherein the length of the antisense strand is 20 nucleotides.

E132. The method of E130, wherein the length of the antisense strand is 21 nucleotides.

E133. The method of E130, wherein the length of the antisense strand is 22 nucleotides.

E134. The method of E130, wherein the length of the antisense strand is 23 nucleotides.

E135. The method of E130, wherein the length of the antisense strand is 24 nucleotides.

E136. The method of E130, wherein the length of the antisense strand is 25 nucleotides.

E137. The method of E129, wherein the length of the antisense strand is 26 nucleotides.

E138. The method of E129, wherein the length of the antisense strand is 27 nucleotides.

E139. The method of E129, wherein the length of the antisense strand is 28 nucleotides.

E140. The method of E129, wherein the length of the antisense strand is 29 nucleotides.

E141. The method of E129, wherein the length of the antisense strand is 30 nucleotides.

E142. The method of any one of E81-E141, wherein the length of the sense strand is between 12 and 30 nucleotides.

E143. The method of E142, wherein the length of the sense strand is 14 nucleotides.

E144. The method of E142, wherein the length of the sense strand is 15 nucleotides.

E145. The method of E142, wherein the length of the sense strand is 16 nucleotides

E146. The method of E142, wherein the length of the sense strand is 17 nucleotides.

E147. The method of E142, wherein the length of the sense strand is 18 nucleotides.

E148. The method of E142, wherein the length of the sense strand is 19 nucleotides.

E149. The method of E142, wherein the length of the sense strand is 20 nucleotides.

E150. The method of E142, wherein the length of the sense strand is 21 nucleotides.

E151. The method of E142, wherein the length of the sense strand is 22 nucleotides.

E152. The method of E142, wherein the length of the sense strand is 23 nucleotides.

E153. The method of E142, wherein the length of the sense strand is 24 nucleotides.

E154. The method of E142, wherein the length of the sense strand is 25 nucleotides.

E155. The method of E142, wherein the length of the sense strand is 26 nucleotides.

E156. The method of E142, wherein the length of the sense strand is 27 nucleotides.

E157. The method of E142, wherein the length of the sense strand is 28 nucleotides.

E158. The method of E142, wherein the length of the sense strand is 29 nucleotides.

E159. The method of E142, wherein the length of the sense strand is 30 nucleotides.

E160. The method of any one of E1-E159, wherein the therapeutic oligonucleotide is administered in the form of an aqueous solution or in the form of a suspension.

E161. The method of any one of E1-E160, wherein the therapeutic oligonucleotide is administered to the circulatory system (e.g., systemically).

E162. The method of any one of E1-E160, wherein the therapeutic oligonucleotide is administered to the central nervous system.

E163. The method of any one of E1-E160, wherein the therapeutic oligonucleotide is administered directly to the cerebral spinal fluid of the subject, optionally wherein the therapeutic oligonucleotide is administered intrathecally, intracerebroventricularly, intrastriatally, or by intra-cisterna magna injection via catheterization.

E164. The method of any one of E1-E160, wherein the therapeutic oligonucleotide is administered directly to the spinal cord of the subject, optionally wherein the therapeutic oligonucleotide is administered intrathecally, intracerebroventricularly, intrastriatally, or by intra-cisterna magna injection by catheterization.

E165. The method of E1-E160, wherein the therapeutic oligonucleotide is administered directly to the brain parenchyma of the subject.

E166. The method of E165, wherein the therapeutic oligonucleotide being administered to the brain is specifically administered to the cortex, cerebellum, basal ganglia, or other brain structure.

E167. The method of E166, wherein the therapeutic oligonucleotide being administered to the basal ganglia is specifically administered to the caudate, putamen, thalamus, globus pallidus, or substantia nigra.

E168. The method of any one of E1-E160, wherein the therapeutic oligonucleotide is administered intrathecally, intracerebroventricularly, intrastriatally, or by intra-cisterna magna injection via catheterization.

E169. The method of E 168, wherein the therapeutic oligonucleotide is administered intrathecally.

E170. The method of E168, wherein the therapeutic oligonucleotide is administered intracerebroventricularly.

E171. The method of any one of E1-E170, wherein the administering of the therapeutic oligonucleotide to the subject results in silencing of a gene or splice isoform of a gene in the subject.

E172. The method of E171, wherein silencing of a gene comprises silencing of a positive regulator of a gene for which increased expression and/or activity, relative to the level of expression and/or activity observed in a reference subject, is associated with a disease state.

E173. The method of E171, wherein silencing of a gene comprises silencing of a negative regulator of a gene for which decreased expression and/or activity, relative to the level of expression and/or activity observed in a reference subject, is associated with a disease state.

E174. The method of any one of E171-E173, wherein silencing of a gene comprises silencing of a gene or a splice isoform of a gene for which overexpression of the gene or splice isoform of the gene, relative to the expression of the gene or splice isoform of the gene in a reference subject, is associated with a disease state.

E175. The method of any one of E171-E174, wherein the gene or splice isoform of the gene is transcriptionally expressed in the central nervous system of the subject.

E176. The method of any one of E171-E175, wherein the silencing of the gene or splice isoform of the gene is used to treat a subject diagnosed with a disease of the central nervous system.

E177. The method of E176, wherein the disease is Huntington's disease, Parkinson's disease, Alzheimer's disease, amyotrophic lateral sclerosis (ALS), dementia with Lewy bodies (DLB), pure autonomic failure, Lewy body dysphagia, incidental Lewy body disease (ILBD), inherited Lewy body disease, olivopontocerebellar atrophy (OPCA), striatonigral degeneration, Shy-Drager syndrome, epilepsy or an epilepsy syndrome, a prion disease, or a pain disorder.

E178. The method of any one of E81-E177, wherein the antisense strand has complementarity sufficient to hybridize a portion of a gene selected from the group consisting of ABCA7, ABI3, ADAM10, APOC1, APOE, AXL, BIN1, C1QA, C3, C90RF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, HTT, IFIT1, IFIT3, IFITM3, IFNAR1, IFNAR2, IGF1, I10RA, IL1A, IL1B, IL1 RAP, INPP5D, ITGAM, ITGAX, KCNT1, LILRB4, LPL, MAPT, MEF2C, MMP12, MS4A4A, MS4A6A, MSH3, NLRP3, NME8, NOS2, PICALM, PILRA, PLCG2, PRNP, PTK2B, SCIMP, SCN9A, SLC24A4, SNCA, SORL1, SPI1, SPP1, SPPL2A, TBK1, TNF, TREM2, TREML2, TYROBP, and ZCWPW1.

E179. The method of E178, wherein the gene is selected from the group consisting of HTT, MAPT, SNCA, C90RF72, APOE, SCN9A, KCNT1, PRNP, and MSH3.

E180. The method of E179, wherein the gene is HTT.

E181. The method of E179, wherein the gene is MAPT.

E182. The method of E179, wherein the gene is SNCA.

E183. The method of E179, wherein the gene is C90RF72.

E184. The method of E179, wherein the gene is APOE.

E185. The method of E179, wherein the gene is SCN9A.

E186. The method of E179, wherein the gene is KCNT1.

E187. The method of E179, wherein the gene is PRNP.

E188. The method of E179, wherein the gene is MSH3 E189. The method of any one of E1-E188, wherein the subject is a human.

E190. The method of any one of E1-E189, wherein the molar ratio of therapeutic oligonucleotide to the one or more divalent cations is from 1:10 to 1:100.

E191. The method of E190, wherein the molar ratio of therapeutic oligonucleotide to the one or more divalent cations is from 1:10 to 1:50 E192. The method of E191, wherein the molar ratio of therapeutic oligonucleotide to the one or more divalent cations is from 1:18 to 1:38.

E193. The method of E192, wherein the molar ratio of therapeutic oligonucleotide to the one or more divalent cations is from 1:20 to 1:25, optionally wherein the molar ratio of therapeutic oligonucleotide to the one or more divalent cations is 1:20.

E194. The method of E193, wherein the molar ratio of therapeutic oligonucleotide to the one or more divalent cations is 1:25.

E195. The method of any one of E1-E194, wherein the concentration of the one or more divalent cations is from 10 mM to 150 mM.

E196. The method of E195, wherein the concentration of the one or more divalent cations is from 20 mM to 150 mM.

E197. The method of E196, wherein the concentration of the one or more divalent cations is from 20 mM to 100 mM.

E198. The method of E196, wherein the concentration of the one or more divalent cations is from 25 mM to 150 mM

E199. The method of E198, wherein the concentration of the one or more divalent cations is from 25 mM to 100 mM

E200. The method of E199, wherein the concentration of the one or more divalent cations is from 30 mM to 90 mM.

E201. The method of E200, wherein the concentration of the one or more divalent cations is from 35 mM to 85 mM

E202. The method of E201, wherein the concentration of the one or more divalent cations is from 35 mM to 75 mM.

E203. The method of E202, wherein the concentration of the one or more divalent cations is from 40 mM to 70 mM.

E204. The method of E203, wherein the concentration of the one or more divalent cations is from 40 mM to 65 mM

E205. The method of E204, wherein the concentration of the one or more divalent cations is from 40 mM to 60 mM

E206. The method of E205, wherein the concentration of the one or more divalent cations is from 40 mM to 50 mM.

E207. The method of any one of E1-E206, wherein the therapeutic oligonucleotide comprises one or more atoms having a negative charge and the divalent cation comprises two positive charges, and wherein the ratio of negative charge to positive charge is from 0.75 to 7.5, optionally wherein the ratio of negative charge to positive charge is from 1.0 to 2.0.

E208. The method of E207, wherein the ratio of negative to positive charge is from 0.75 to 6.5.

E209. The method of E208, wherein the ratio of negative to positive charge is from 0.75 to 5.5

E210. The method of E209, wherein the ratio of negative to positive charge is from 0.75 to 4.5.

E211. The method of E210, wherein the ratio of negative to positive charge is from 0.75 to 3.5.

E212. The method of E211, wherein the ratio of negative to positive charge is from 0.75 to 2.5.

E213. The method of E212, wherein the ratio of negative to positive charge is from 0.75 to 1.5.

E214. The method of E213, wherein the ratio of negative to positive charge is from 0.75 to 1.

E215. The method of E208, wherein the ratio of negative to positive charge is from 1 to 7.5.

E216. The method of E215, wherein the ratio of negative to positive charge is from 1.5 to 7.5.

E217. The method of E216, wherein the ratio of negative to positive charge is from 2.5 to 7.5.

E218. The method of E217, wherein the ratio of negative to positive charge is from 3.5 to 7.5.

E219. The method of E218, wherein the ratio of negative to positive charge is from 4.5 to 7.5.

E220. The method of E219, wherein the ratio of negative to positive charge is from 5.5 to 7.5.

E221. The method of E220, wherein the ratio of negative to positive charge is from 6.5 to 7.5.

E222. A therapeutic oligonucleotide (e.g., siRNA, shRNA, miRNA, gRNA, ASO) formulated as a salt comprising one or more divalent cations, optionally wherein the therapeutic oligonucleotide is an interfering RNA molecule (e.g., siRNA, shRNA, miRNA).

E223. The therapeutic oligonucleotide of E222, where the therapeutic oligonucleotide is a siRNA molecule.

E224. The therapeutic oligonucleotide of E222, wherein the siRNA molecule comprises a plurality of cationic binding sites that are partially or fully saturated by the one or more divalent cations.

E225. The therapeutic oligonucleotide of E224, wherein the degree of saturation of the cationic binding sites by the one or more divalent cations is from about 10% to about 100%.

E226. The therapeutic oligonucleotide of E225, wherein the degree of saturation of the cationic binding sites by the one or more divalent cations is from about 20% to about 100%.

E227. The therapeutic oligonucleotide of E226, wherein the degree of saturation of the cationic binding sites by the one or more divalent cations is from about 30% to about 100%.

E228. The therapeutic oligonucleotide of E227, wherein the degree of saturation of the cationic binding sites by the one or more divalent cations is from about 40% to about 100%.

E229. The therapeutic oligonucleotide of E228, wherein the degree of saturation of the cationic binding sites by the one or more divalent cations is from about 50% to about 100%.

E230. The therapeutic oligonucleotide of E229, wherein the degree of saturation of the cationic binding sites by the one or more divalent cations is from about 60% to about 100%.

E231. The therapeutic oligonucleotide of E230, wherein the degree of saturation of the cationic binding sites by the one or more divalent cations is from about 70% to about 100%.

E232. The therapeutic oligonucleotide of E231, wherein the degree of saturation of the cationic binding sites by the one or more divalent cations is from about 80% to about 100%.

E233. The therapeutic oligonucleotide of E232, wherein the degree of saturation of the cationic binding sites by the one or more divalent cations is from about 90% to about 100%.

E234. The therapeutic oligonucleotide of any one of E224-E233, wherein the cationic binding site is located within an internucleoside linkage, optionally wherein the internucleoside linkage is selected from a phosphodiester linkage and a phosphorothioate linkage.

E235. The therapeutic oligonucleotide of any one of E222-E234, wherein the one or more divalent cations is characterized by an ionic radius of from about 30 picometers to about 150 picometers.

E236. The therapeutic oligonucleotide of any one of E235, wherein the one or more divalent cations is characterized by an ionic radius of from about 30 picometers to about 140 picometers.

E237. The therapeutic oligonucleotide of any one of E236, wherein the one or more divalent cations is characterized by an ionic radius of from about 40 picometers to about 130 picometers.

E238. The therapeutic oligonucleotide of any one of E237, wherein the one or more divalent cations is characterized by an ionic radius of from about 50 picometers to about 120 picometers.

E239. The therapeutic oligonucleotide of any one of E238, wherein the one or more divalent cations is characterized by an ionic radius of from about 60 picometers to about 110 picometers.

E240. The therapeutic oligonucleotide of any one of E239, wherein the one or more divalent cations is characterized by an ionic radius of from about 60 picometers to about 100 picometers.

E241. The therapeutic oligonucleotide of any one of E240, wherein the one or more divalent cations is characterized by an ionic radius of from about 60 picometers to about 90 picometers.

E242. The therapeutic oligonucleotide of any one of E222-E234, wherein the one or more divalent cations comprise Ba²⁺, Be²⁺, Ca²⁺, Cu²⁺, Mg²⁺, Mn²⁺, Ni²⁺, Zn²⁺, or a combination thereof.

E243. The therapeutic oligonucleotide of E242, wherein the one or more divalent cations comprise Ba²⁺.

E244. The therapeutic oligonucleotide of E242 or E243, wherein the one or more divalent cations comprise Be²⁺.

E245. The therapeutic oligonucleotide of any one of E242-E244, wherein the one or more divalent cations comprise Ca²⁺.

E246. The therapeutic oligonucleotide of any one of E242-E245, wherein the one or more divalent cations comprise Cu²⁺.

E247. The therapeutic oligonucleotide of any one of E242-E246, wherein the one or more divalent cations comprise Mg²⁺.

E248. The therapeutic oligonucleotide of any one of E242-E247, wherein the one or more divalent cations comprise Mn²⁺ E249. The therapeutic oligonucleotide of any one of E242-E248, wherein the one or more divalent cations comprise Ni²⁺.

E250. The therapeutic oligonucleotide of any one of E242-E249, wherein the one or more divalent cations comprise Zn²⁺.

E251. The therapeutic oligonucleotide of any one of E242-E250, wherein the one or more divalent cations comprise Ca²⁺ and Mg²⁺.

E252. The therapeutic oligonucleotide of any one of E222-E251, wherein the one or more divalent cations comprise a hard Lewis acid.

E253. The therapeutic oligonucleotide of any one of E222-E252, wherein the one or more divalent cations displaces water from a cationic binding site of the siRNA molecule.

E254. The therapeutic oligonucleotide of any one of E222-E253, wherein the siRNA molecule is branched, optionally wherein the siRNA molecule is di-branched, tri-branched, or tetra-branched.

E255. The therapeutic oligonucleotide of E254, wherein the siRNA molecule is di-branched.

E256. The therapeutic oligonucleotide of E254, wherein the siRNA molecule is tri-branched.

E257. The therapeutic oligonucleotide of E254, wherein the siRNA molecule is tetra-branched.

E258. The therapeutic oligonucleotide of E254 or E255, wherein the di-branched siRNA molecule is represented by any one of Formulas I-III:

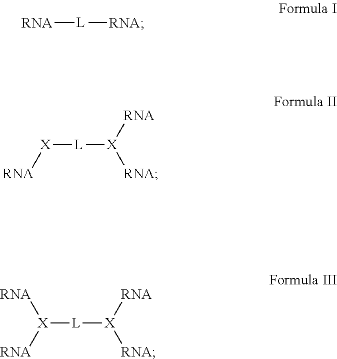

wherein each RNA is, independently, an siRNA molecule, L is a linker, and each X, independently, represents a branch point moiety.

E259. The therapeutic oligonucleotide of E258, wherein the di-branched siRNA molecule is represented by Formula I.

E260. The therapeutic oligonucleotide of E258, wherein the di-branched siRNA molecule is represented by Formula II.

E261. The therapeutic oligonucleotide of E258, wherein the di-branched siRNA molecule is represented by Formula III.

E262. The therapeutic oligonucleotide of E254 or E256, wherein the tri-branched siRNA molecule is represented by any one of Formulas IV-VII:

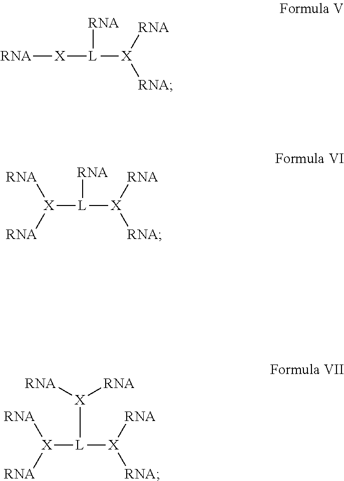

wherein each RNA is, independently, an siRNA molecule, L is a linker, and each X, independently, represents a branch point moiety.

E263. The therapeutic oligonucleotide of E262, wherein the tri-branched siRNA molecule is represented by Formula IV.

E264. The therapeutic oligonucleotide of E262, wherein the tri-branched siRNA molecule is represented by Formula V.

E265. The therapeutic oligonucleotide of E262, wherein the tri-branched siRNA molecule is represented by Formula VI.

E266. The therapeutic oligonucleotide of E262, wherein the tri-branched siRNA molecule is represented by Formula VII.

E267. The therapeutic oligonucleotide of E254 or E257, wherein the tetra-branched siRNA molecule is represented by any one of Formulas VIII-XII:

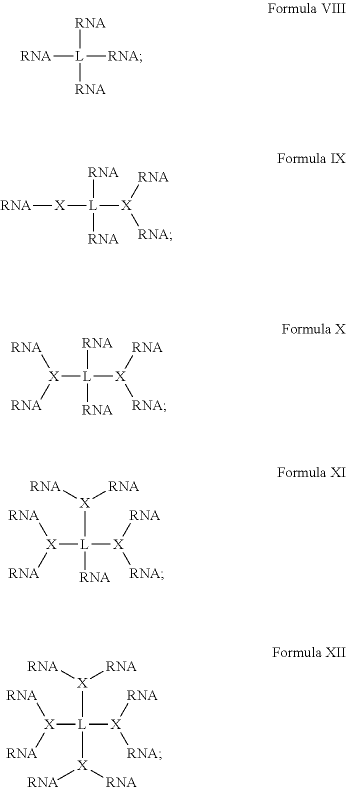

wherein each RNA is, independently, an siRNA molecule, L is a linker, and each X, independently, represents a branch point moiety.

E268. The therapeutic oligonucleotide of E267, wherein the tetra-branched siRNA molecule is represented by Formula VIII.

E269. The therapeutic oligonucleotide of E267, wherein the tetra-branched siRNA molecule is represented by Formula IX.

E270. The therapeutic oligonucleotide of E267, wherein the tetra-branched siRNA molecule is represented by Formula X.

E271. The therapeutic oligonucleotide of E267, wherein the tetra-branched siRNA molecule is represented by Formula XI.

E272. The therapeutic oligonucleotide of E267, wherein the tetra-branched siRNA molecule is represented by Formula XII.

E273. The therapeutic oligonucleotide of any one of E258-E272, wherein the linker is selected from a group consisting of one or more contiguous subunits of an ethylene glycol (e.g., polyethylene glycol (PEG), such as, e.g., triethylene glycol (TrEG) or tetraethylene glycol (TEG)), alkyl, carbohydrate, block copolymer, peptide, RNA, and DNA.

E274. The therapeutic oligonucleotide of E273, wherein the linker is an ethylene glycol oligomer.

E275. The therapeutic oligonucleotide of E274, wherein the ethylene glycol oligomer is a PEG.

E276. The therapeutic oligonucleotide of E275, wherein the PEG a TrEG.

E277. The therapeutic oligonucleotide of E276, wherein the PEG is a TEG.

E278. The therapeutic oligonucleotide of E273, wherein the linker is an alkyl oligomer.

E279. The therapeutic oligonucleotide of E273, wherein the linker is a carbohydrate oligomer.

E280. The therapeutic oligonucleotide of E273, wherein the linker is a block copolymer.

E281. The therapeutic oligonucleotide of E273, wherein the linker is a peptide oligomer.

E282. The therapeutic oligonucleotide of E273, wherein the linker is an RNA oligomer.

E283. The therapeutic oligonucleotide of E273, wherein the linker is a DNA oligomer.

E284. The therapeutic oligonucleotide of any one of E273-E283, wherein the oligomer or copolymer contains 2 to 20 contiguous subunits.

E285. The therapeutic oligonucleotide of E284, wherein oligomer or copolymer contains 4 to 18 contiguous subunits.

E286. The therapeutic oligonucleotide of E284, wherein oligomer or copolymer contains 6 to 16 contiguous subunits.

E287. The therapeutic oligonucleotide of E286, wherein oligomer or copolymer contains 8 to 14 contiguous subunits.

E288. The therapeutic oligonucleotide of E287, wherein oligomer or copolymer contains 10 to 12 contiguous subunits.

E289. The therapeutic oligonucleotide of E243, wherein the linker attaches one or more (e.g., 1, 2, or more) siRNA molecules by way of a covalent bond-forming moiety.

E290. The therapeutic oligonucleotide of E289, wherein the covalent bond-forming moiety is selected from the group consisting of an alkyl, ester, amide, carbamate, phosphonate, phosphate, phosphorothioate, phosphoroamidate, triazole, urea, and formacetal.

E291. The therapeutic oligonucleotide of E273, wherein the linker includes a structure of Formula L1:

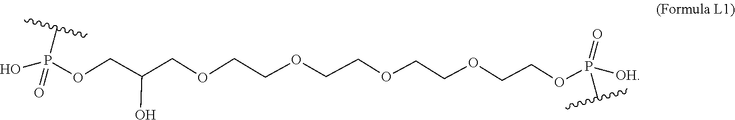 [SMILES: *P(=O)(O)OCCOCCOCCOCCOCC(O)COP(*)(=O)O]

E292. The therapeutic oligonucleotide of E273, wherein the linker includes a structure of Formula L2:

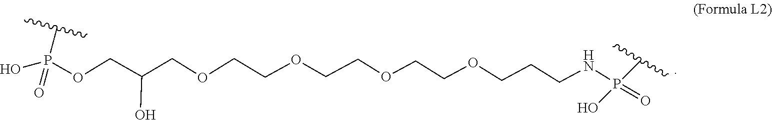 [SMILES: *P(=O)(O)NCCCOCCOCCOCCOCC(O)COP(*)(=O)O]

E293. The therapeutic oligonucleotide of E273, wherein the linker includes a structure of Formula L3:

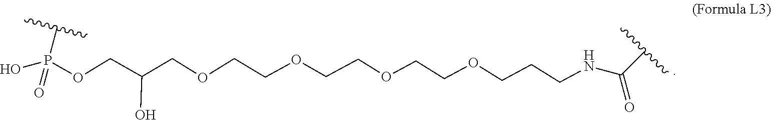 [SMILES: *C(=O)NCCCOCCOCCOCCOCC(O)COP(*)(=O)O]

E294. The therapeutic oligonucleotide of E273, wherein the linker includes a structure of Formula L4:

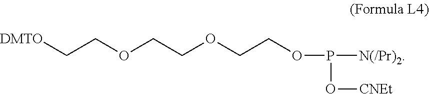

E295. The therapeutic oligonucleotide of E273, wherein the linker includes a structure of Formula L5:

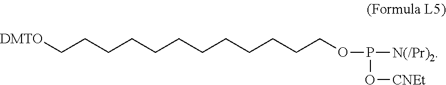

E296. The therapeutic oligonucleotide of E273, wherein the linker includes a structure of Formula L6:

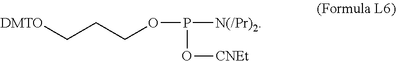

E297. The therapeutic oligonucleotide of E273, wherein the linker includes a structure of Formula L7:

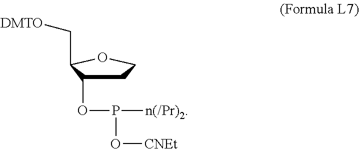

E298. The therapeutic oligonucleotide of E273, wherein the linker includes a structure of Formula L8:

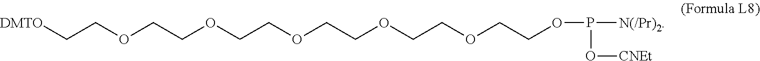

E299. The therapeutic oligonucleotide of E273, wherein the linker includes a structure of Formula L9:

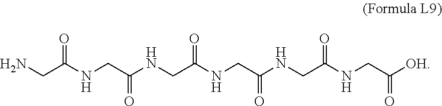 [SMILES: NCC(=O)NCC(=O)NCC(=O)NCC(=O)NCC(=O)NCC(=O)O]

E300. The therapeutic oligonucleotide of any one of E222-E299, wherein the siRNA molecule comprises an antisense strand and a sense strand having complementarity to the antisense strand.

E301. The therapeutic oligonucleotide of E300, wherein the antisense strand and sense strand comprises alternating 2′-O-methyl and 2′-fluoro ribonucleosides.

E302. The therapeutic oligonucleotide of E300 or E301, wherein the antisense strand has the following formula, in the 5′-to-3′ direction:

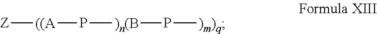

wherein Z is a 5′ phosphorus stabilizing moiety;
each A is, independently, a 2′-O-methyl (2′-O-Me) ribonucleoside;
each B is, independently, a 2′-fluoro (2′-F) ribonucleoside;
each P is, independently, an internucleoside linkage selected from a phosphodiester linkage and a phosphorothioate linkage;
n is an integer from 1 to 5;
m is an integer from 1 to 5; and
q is an integer between 1 and 30.

E303. The therapeutic oligonucleotide of E302, wherein n is from 1 to 4.

E304. The therapeutic oligonucleotide of E302, wherein n is from 1 to 3.

E305. The therapeutic oligonucleotide of E302, wherein n is from 1 to 2.

E306. The therapeutic oligonucleotide of E302, wherein n is 1.

E307. The therapeutic oligonucleotide of E302, wherein n is 2.

E308. The therapeutic oligonucleotide of E302, wherein n is 3.

E309. The therapeutic oligonucleotide of E302, wherein n is 4.

E310. The therapeutic oligonucleotide of E302, wherein n is 5.

E311. The therapeutic oligonucleotide of any one of E302-E310, wherein m is from 1 to 4.

E312. The therapeutic oligonucleotide of E311, wherein m is from 1 to 3.

E313. The therapeutic oligonucleotide of E311, wherein m is from 1 to 2.

E314. The therapeutic oligonucleotide of E311, wherein m is 1.

E315. The therapeutic oligonucleotide of E311, wherein m is 2.

E316. The therapeutic oligonucleotide of E311, wherein m is 3.

E317. The therapeutic oligonucleotide of E311, wherein m is 4.

E318. The therapeutic oligonucleotide of E311, wherein m is 5.

E319. The therapeutic oligonucleotide E300 or E301, wherein the antisense strand comprises a structure represented by Formula I, in the 5′-to-3′ direction:

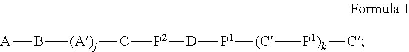

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

E320. The therapeutic oligonucleotide of claim E319, wherein the antisense strand comprises a structure represented by Formula A1, wherein Formula A1 is, in the 5′-to-3′ direction:

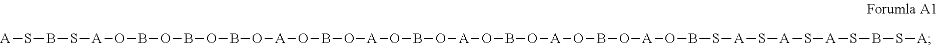

wherein A represents a 2′-O-Me ribonucleoside, B represents a 2′-F ribonucleoside, O represents a phosphodiester internucleoside linkage, and S represents a phosphorothioate internucleoside linkage.

E321. The therapeutic oligonucleotide of E319 wherein the antisense strand comprises a structure represented by Formula II, wherein Formula II is, in the 5′-to-3′ direction:

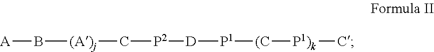

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

E322. The therapeutic oligonucleotide of E321, the antisense strand comprises a structure represented by Formula A2, wherein Formula A2 is, in the 5′-to-3′ direction:

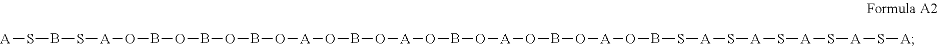

wherein A represents a 2′-O-Me ribonucleoside, B represents a 2′-F ribonucleoside, O represents a phosphodiester internucleoside linkage, and S represents a phosphorothioate internucleoside linkage.

E323. The therapeutic oligonucleotide of any one of E300-E322, wherein the sense strand comprises a structure represented by Formula III, wherein Formula III is, in the 5′-to-3′ direction:

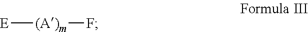

wherein E is represented by the formula (C—P¹)₂;
F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D;
A′, C, D, P¹, and P² are as defined in Formula II; and
m is an integer from 1 to 7.

E324. The therapeutic oligonucleotide of E323, wherein the sense strand comprises a structure represented by Formula S1 wherein Formula S1 is in the 5′-to-3′ direction:

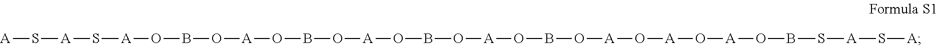

wherein A represents a 2′-O-Me ribonucleoside, B represents a 2′-F ribonucleoside, O represents a phosphodiester internucleoside linkage, and S represents a phosphorothioate internucleoside linkage.

E325. The therapeutic oligonucleotide of E323, wherein the sense strand comprises a structure represented by Formula S2, wherein Formula S2 is, in the 5′-to-3′ direction:

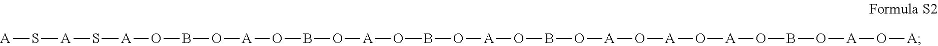

wherein A represents a 2′-O-Me ribonucleoside, B represents a 2′-F ribonucleoside, O represents a phosphodiester internucleoside linkage, and S represents a phosphorothioate internucleoside linkage.

E326. The therapeutic oligonucleotide of E323, wherein the sense strand comprises a structure represented by Formula S3, wherein Formula S3 is, in the 5′-to-3′ direction:

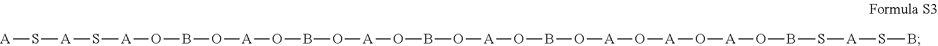

wherein A represents a 2′-O-Me ribonucleoside, B represents a 2′-F ribonucleoside, O represents a phosphodiester internucleoside linkage, and S represents a phosphorothioate internucleoside linkage.

E327. The therapeutic oligonucleotide of E323, wherein the sense strand comprises a structure represented by Formula S4, wherein Formula S4 is, in the 5′-to-3′ direction:

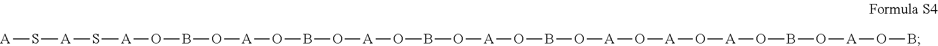

wherein A represents a 2′-O-Me ribonucleoside, B represents a 2′-F ribonucleoside, O represents a phosphodiester internucleoside linkage, and S represents a phosphorothioate internucleoside linkage.

E328. The therapeutic oligonucleotide of any one of E300, E301, and E323-E327, wherein the antisense strand comprises a structure represented by Formula IV, wherein Formula IV is, in the 5′-to-3′ direction:

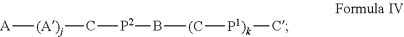

wherein A is represented by the formula C—P¹-D-P¹;
each A′ is represented by the formula C—P²-D-P²;
B is represented by the formula D-P¹—C—P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

E329. The therapeutic oligonucleotide of E328, wherein the antisense strand comprises a structure represented by Formula A3, wherein Formula A3 is, in the 5′-to-3′ direction:

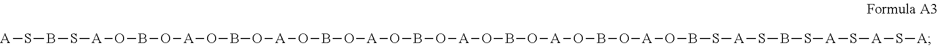

wherein A represents a 2′-O-Me ribonucleoside, B represents a 2′-F ribonucleoside, O represents a phosphodiester internucleoside linkage, and S represents a phosphorothioate internucleoside linkage.

E330. The therapeutic oligonucleotide of E300-E292, E328, and E329, wherein the sense strand comprises a structure represented by Formula V, wherein Formula V is, in the 5′-to-3′ direction:

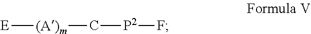

wherein E is represented by the formula (C-P¹)₂;
F is represented by the formula D-P¹-C-P¹-C, D-P²-C-P²-C, D-P¹-C-P¹-D, or D-P²-C-P²-C;
A′, C, D, P¹ and P² are as defined in Formula IV; and
m is an integer from 1 to 7.

E331. The therapeutic oligonucleotide of E330, wherein the sense strand comprises a structure represented by Formula S5, wherein Formula S5 is, in the 5′-to-3′ direction:

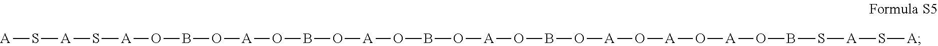

wherein A represents a 2′-O-Me ribonucleoside, B represents a 2′-F ribonucleoside, O represents a phosphodiester internucleoside linkage, and S represents a phosphorothioate internucleoside linkage.

E332. The therapeutic oligonucleotide of E330, wherein the sense strand comprises a structure represented by Formula S6, wherein Formula S6 is, in the 5′-to-3′ direction:

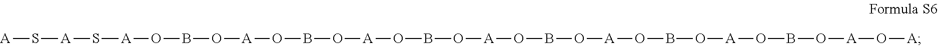

wherein A represents a 2′-O-Me ribonucleoside, B represents a 2′-F ribonucleoside, O represents a phosphodiester internucleoside linkage, and S represents a phosphorothioate internucleoside linkage.

E333. The therapeutic oligonucleotide of E330, wherein the sense strand comprises a structure represented by Formula S7, wherein Formula S7 is, in the 5′-to-3′ direction:

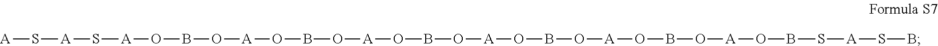

wherein A represents a 2′-O-Me ribonucleoside, B represents a 2′-F ribonucleoside, O represents a phosphodiester internucleoside linkage, and S represents a phosphorothioate internucleoside linkage.

E334. The therapeutic oligonucleotide of E330, wherein the sense strand comprises a structure represented by Formula S8, wherein Formula S8 is, in the 5′-to-3′ direction:

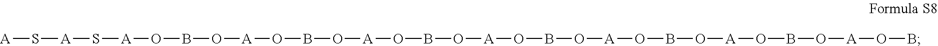

wherein A represents a 2′-O-Me ribonucleoside, B represents a 2′-F ribonucleoside, O represents a phosphodiester internucleoside linkage, and S represents a phosphorothioate internucleoside linkage.

E335. The therapeutic oligonucleotide of any one of E300, E301, E23-E327, and E330-E334, wherein the antisense strand comprises a structure represented by Formula VI, wherein Formula VI is, in the 5′-to-3′ direction:

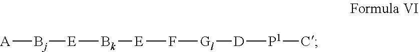

wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7;
k is an integer from 1 to 7; and
l is an integer from 1 to 7.

E336. The therapeutic oligonucleotide of E335, wherein the antisense strand comprises a structure represented by Formula A4, wherein Formula A4 is, in the 5′-to-3′ direction:

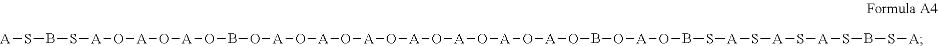

wherein A represents a 2′-O-Me ribonucleoside, B represents a 2′-F ribonucleoside, O represents a phosphodiester internucleoside linkage, and S represents a phosphorothioate internucleoside linkage.

E337. The therapeutic oligonucleotide of any one of claims any one of claims E300-E322, E328, E329, E335, and E336 wherein the sense strand comprises a structure represented by Formula VII, wherein Formula VII is, in the 5′-to-3′ direction:

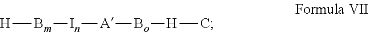

wherein A′ is represented by the formula C—P²-D-P²;
each H is represented by the formula (C—P¹)₂;
each I is represented by the formula (D-P²);
B, C, D, P¹ and P² are as defined in Formula VI;
m is an integer from 1 to 7;
n is an integer from 1 to 7; and
is an integer from 1 to 7.

E338. The therapeutic oligonucleotide of E337, wherein the sense strand comprises a structure represented by Formula s9, wherein Formula S9 is, in the 5′-to-3′ direction:

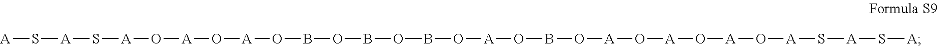

wherein A represents a 2′-O-Me ribonucleoside, B represents a 2′-F ribonucleoside, O represents a phosphodiester internucleoside linkage, and S represents a phosphorothioate internucleoside linkage.

E339. The therapeutic oligonucleotide of any one of E300-E338, wherein the antisense strand further comprises a 5′-phosphorus stabilizing moiety at the 5′ end of the antisense strand.

E340. The therapeutic oligonucleotide of any one of E300-E339, wherein the sense strand further comprises a 5′-phosphorus stabilizing moiety at the 5′ end of the sense strand.

E341. The therapeutic oligonucleotide of any one of E302-E318, E338, and E339, wherein the 5′-phosphorus stabilizing moiety is represented in any one of Formula IX-XVI:

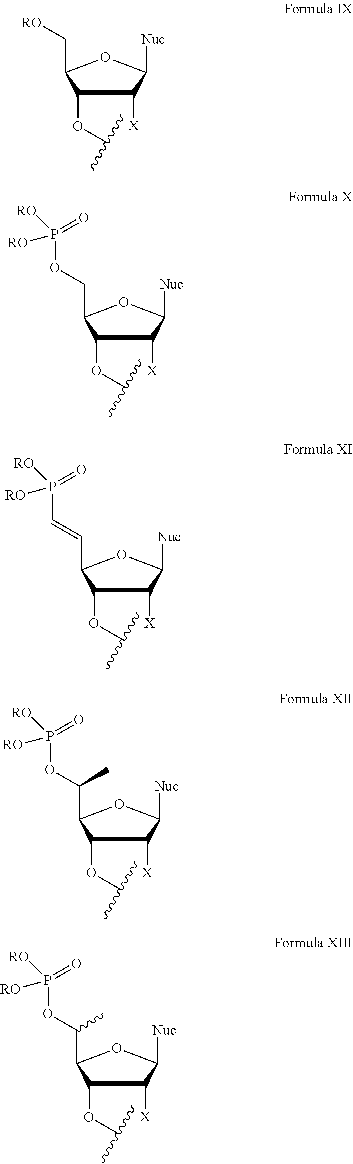

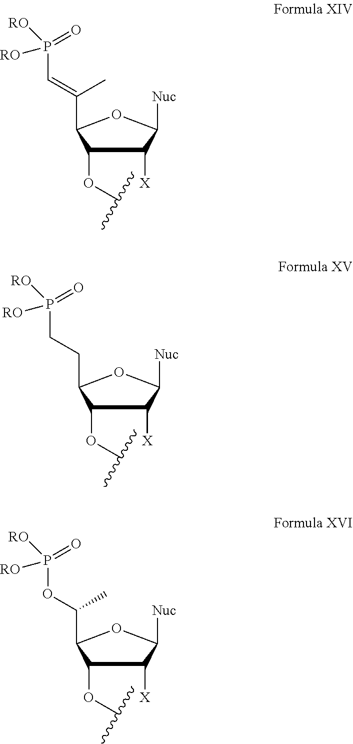

wherein Nuc represents a nucleobase selected from the group consisting of adenine, uracil, guanine, thymine, and cytosine, and R represents optionally substituted alkyl, optionally substituted alkenyl, optionally substituted alkynyl, phenyl, benzyl, hydroxy, or hydrogen.

E342. The therapeutic oligonucleotide of E341, wherein the 5′-phosphorus stabilizing moiety is (E)-vinylphosphonate represented in Formula XVI.

E343. The therapeutic oligonucleotide of any one of E301-E342, wherein at least 50% of the ribonucleosides are 2′-O-Me ribonucleoside.

E344. The therapeutic oligonucleotide of any one of E301-E343, wherein at least 60% of the ribonucleosides are 2′-O-Me ribonucleoside.

E345. The therapeutic oligonucleotide of any one of E301-E344, wherein at least 70% of the ribonucleosides are 2′-O-Me ribonucleoside.

E346. The therapeutic oligonucleotide of any one of E301-E345, wherein at least 80% of the ribonucleosides are 2′-O-Me ribonucleoside.

E347. The therapeutic oligonucleotide of any one of E301-E346, wherein at least 90% of the ribonucleosides are 2′-O-Me ribonucleoside.

E348. The therapeutic oligonucleotide of any one of E300-E347, wherein the length of the antisense strand is between 10 and 30 nucleotides.

E349. The therapeutic oligonucleotide of any one of E300-E348, wherein the length of the antisense strand is between 15 and 25 nucleotides.

E350. The therapeutic oligonucleotide of E349, wherein the length of the antisense strand is 20 nucleotides.

E351. The therapeutic oligonucleotide of E349, wherein the length of the antisense strand is 21 nucleotides.

E352. The therapeutic oligonucleotide of E349, wherein the length of the antisense strand is 22 nucleotides.

E353. The therapeutic oligonucleotide of E349, wherein the length of the antisense strand is 23 nucleotides.

E354. The therapeutic oligonucleotide of E349, wherein the length of the antisense strand is 24 nucleotides.

E355. The therapeutic oligonucleotide of E349, wherein the length of the antisense strand is 25 nucleotides.

E356. The therapeutic oligonucleotide of E348, wherein the length of the antisense strand is 26 nucleotides.

E357. The therapeutic oligonucleotide of E348, wherein the length of the antisense strand is 27 nucleotides.

E358. The therapeutic oligonucleotide of E348, wherein the length of the antisense strand is 28 nucleotides.

E359. The therapeutic oligonucleotide of E348, wherein the length of the antisense strand is 29 nucleotides.

E360. The therapeutic oligonucleotide of E348, wherein the length of the antisense strand is 30 nucleotides.

E361. The therapeutic oligonucleotide of any one of E300-E360, wherein the length of the sense strand is between 12 and 30 nucleotides.

E362. The therapeutic oligonucleotide of E361, wherein the length of the sense strand is 14 nucleotides.

E363. The therapeutic oligonucleotide of E361, wherein the length of the sense strand is 15 nucleotides.

E364. The therapeutic oligonucleotide of E361, wherein the length of the sense strand is 16 nucleotides.

E365. The therapeutic oligonucleotide of E361, wherein the length of the sense strand is 17 nucleotides.

E366. The therapeutic oligonucleotide of E361, wherein the length of the sense strand is 18 nucleotides.

E367. The therapeutic oligonucleotide of E361, wherein the length of the sense strand is 19 nucleotides.

E368. The therapeutic oligonucleotide of E361, wherein the length of the sense strand is 20 nucleotides.

E369. The therapeutic oligonucleotide of E361, wherein the length of the sense strand is 21 nucleotides.

E370. The therapeutic oligonucleotide of E361, wherein the length of the sense strand is 22 nucleotides.

E371. The therapeutic oligonucleotide of E361, wherein the length of the sense strand is 23 nucleotides.

E372. The therapeutic oligonucleotide of E361, wherein the length of the sense strand is 24 nucleotides.

E373. The therapeutic oligonucleotide of E361, wherein the length of the sense strand is 25 nucleotides.

E374. The therapeutic oligonucleotide of E361, wherein the length of the sense strand is 26 nucleotides.

E375. The therapeutic oligonucleotide of E361, wherein the length of the sense strand is 27 nucleotides.

E376. The therapeutic oligonucleotide of E361, wherein the length of the sense strand is 28 nucleotides.

E377. The therapeutic oligonucleotide of E361, wherein the length of the sense strand is 29 nucleotides.

E378. The therapeutic oligonucleotide of E361, wherein the length of the sense strand is 30 nucleotides.

E379. The therapeutic oligonucleotide of any one of E222-E378, wherein the siRNA molecule is administered to the central nervous system of the subject in the form of an aqueous solution or in the form of a suspension.

E380. The therapeutic oligonucleotide of any one of E222-E379, wherein administration of the siRNA molecule to the subject results in silencing of a gene or splice isoform of a gene in the subject.

E381. The therapeutic oligonucleotide of E380, wherein silencing of a gene comprises silencing of a positive regulator of a gene for which increased expression and/or activity, relative to the level of expression and/or activity observed in a reference subject, is associated with a disease state.

E382. The therapeutic oligonucleotide of E380, wherein silencing of a gene comprises silencing of a negative regulator of a gene for which decreased expression and/or activity, relative to the level of expression and/or activity observed in a reference subject, is associated with a disease state.

E383. The therapeutic oligonucleotide of any one of E380-E382, wherein silencing of a gene comprises silencing of a gene or a splice isoform of a gene for which overexpression of the gene or splice isoform of the gene, relative to the expression of the gene or splice isoform of the gene in a reference subject, is associated with a disease state.

E384. The therapeutic oligonucleotide of any one of E380-E383, wherein the gene or splice isoform of the gene is transcriptionally expressed in the central nervous system of the subject.

E385. The therapeutic oligonucleotide of any one of E380-E384, wherein the silencing of the gene or splice isoform of a gene is used to treat a subject diagnosed with a disease of the central nervous system.

E386. The therapeutic oligonucleotide of E385, wherein the disease is Huntington's disease, Parkinson's disease, Alzheimer's disease, amyotrophic lateral sclerosis (ALS), dementia with Lewy bodies (DLB), pure autonomic failure, Lewy body dysphagia, incidental Lewy body disease (ILBD), inherited Lewy body disease, olivopontocerebellar atrophy (OPCA), striatonigral degeneration, Shy-Drager syndrome, epilepsy or an epilepsy disorder, a prion disease, or a pain disorder

E387. The therapeutic oligonucleotide of E300-E386, wherein the antisense strand has complementarity sufficient to hybridize a portion of a gene selected from the group consisting of ABCA7, ABI3, ADAM10, APOC1, APOE, AXL, BIN1, C1QA, C3, C90RF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, HTT, IFIT1, IFIT3, IFITM3, IFNAR1, IFNAR2, IGF1, IL10RA, ILlA, IL1B, IL1RAP, INPP5D, ITGAM, ITGAX, KCNT1, LILRB4, LPL, MAPT, MEF2C, MMP12, MS4A4A, MS4A6A, MSH3, NLRP3, NME8, NOS2, PICALM, PILRA, PLCG2, PRNP, PTK2B, SCIMP, SCN9A, SLC24A4, SNCA, SORL1, SPI1, SPP1, SPPL2A, TBK1, TNF, TREM2, TREML2, TYROBP, and ZCWPW1.

E388. The therapeutic oligonucleotide of E387, wherein the gene is selected from the group consisting of HTT, MAPT, SNCA, C90RF72, APOE, SCN9A, KCNT1, PRNP, and MSH3.

E389. The therapeutic oligonucleotide of E388, wherein the gene is HTT.

E390. The therapeutic oligonucleotide of E388, wherein the gene is MAPT.

E391. The therapeutic oligonucleotide of E388, wherein the gene is SNCA.

E392. The therapeutic oligonucleotide of E388, wherein the gene is C90RF72.

E393. The therapeutic oligonucleotide of E388, wherein the gene is APOE.

E394. The therapeutic oligonucleotide of E388, wherein the gene is SCN9A.

E395. The therapeutic oligonucleotide of E388, wherein the gene is KCNT1.

E396. The therapeutic oligonucleotide of E388, wherein the gene is PRNP.

E397. The therapeutic oligonucleotide of E388, wherein the gene is MSH3.

E398. The therapeutic oligonucleotide of any one of E222-E397, wherein the subject is a human.

E399. The therapeutic oligonucleotide of any one E222-E398, wherein the molar ratio of therapeutic oligonucleotide to divalent cation is from 1:10 to 1:100 (e.g., 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, or 1:100).

E400. The therapeutic oligonucleotide of E399, wherein the molar ratio of therapeutic oligonucleotide to divalent cation is from 1:10 to 1:50

E401. The therapeutic oligonucleotide of E400, wherein the molar ratio of therapeutic oligonucleotide to divalent cation is from 1:18 to 1:38.

E402. The therapeutic oligonucleotide of E401, wherein the molar ratio of therapeutic oligonucleotide to divalent cation is from 1:20 to 1:25, optionally wherein the molar ratio of therapeutic oligonucleotide to divalent cation is 1:20.

E403. The therapeutic oligonucleotide of E402, wherein the molar ratio of therapeutic oligonucleotide to divalent cation is 1:25.

E404. The therapeutic oligonucleotide of any one E222-E403, wherein the concentration of the one or more divalent cations is from 10 mM to 150 mM.

E405. The therapeutic oligonucleotide of E404, wherein the concentration of the one or more divalent cations is from 20 mM to 150 mM.

E406. The therapeutic oligonucleotide of E405, wherein the concentration of the one or more divalent cations is from 20 mM to 100 mM.

E407. The therapeutic oligonucleotide of E405, wherein the concentration of the one or more divalent cations is from 25 mM to 150 mM

E408. The therapeutic oligonucleotide of E407, wherein the concentration of the one or more divalent cations is from 25 mM to 100 mM

E409. The therapeutic oligonucleotide of E408, wherein the concentration of the one or more divalent cations is from 30 mM to 90 mM.

E410. The therapeutic oligonucleotide of E409, wherein the concentration of the one or more divalent cations is from 35 mM to 85 mM.

E411. The therapeutic oligonucleotide of E410, wherein the concentration of the one or more divalent cations is from 35 mM to 75 mM.

E412. The therapeutic oligonucleotide of E411, wherein the concentration of the one or more divalent cations is from 40 mM to 70 mM.

E413. The therapeutic oligonucleotide of E412, wherein the concentration of the one or more divalent cations is from 40 mM to 65 mM.

E414. The therapeutic oligonucleotide of E413, wherein the concentration of the one or more divalent cations is from 40 mM to 60 mM.

E415. The therapeutic oligonucleotide of E414, wherein the concentration of the one or more divalent cations is from 40 mM to 50 mM.

E416. The therapeutic oligonucleotide of any one of E222-E398, wherein the therapeutic oligonucleotide comprises one or more atoms having a negative charge and the divalent cation comprises two positive charges, and wherein the ratio of negative charge to positive charge is from 0.75 to 7.5, optionally wherein the ratio of negative charge to positive charge is from 1.0 to 2.0.

E417. The therapeutic oligonucleotide of E416, wherein the ratio of negative to positive charge is from 0.75 to 6.5.

E418. The therapeutic oligonucleotide of E417, wherein the ratio of negative to positive charge is from 0.75 to 5.5

E419. The therapeutic oligonucleotide of E418, wherein the ratio of negative to positive charge is from 0.75 to 4.5.

E420. The therapeutic oligonucleotide of E419, wherein the ratio of negative to positive charge is from 0.75 to 3.5.

E421. The therapeutic oligonucleotide of E420, wherein the ratio of negative to positive charge is from 0.75 to 2.5.

E422. The therapeutic oligonucleotide of E421, wherein the ratio of negative to positive charge is from 0.75 to 1.5.

E423. The therapeutic oligonucleotide of E422, wherein the ratio of negative to positive charge is from 0.75 to 1.

E424. The therapeutic oligonucleotide of E416, wherein the ratio of negative to positive charge is from 1 to 7.5.

E425. The therapeutic oligonucleotide of E424, wherein the ratio of negative to positive charge is from 1.5 to 7.5.

E426. The therapeutic oligonucleotide of E425, wherein the ratio of negative to positive charge is from 2.5 to 7.5.

E427. The therapeutic oligonucleotide of E426, wherein the ratio of negative to positive charge is from 3.5 to 7.5.

E428. The therapeutic oligonucleotide of E427, wherein the ratio of negative to positive charge is from 4.5 to 7.5.

E429. The therapeutic oligonucleotide of E428, wherein the ratio of negative to positive charge is from 5.5 to 7.5.

E430. The therapeutic oligonucleotide of E429, wherein the ratio of negative to positive charge is from 6.5 to 7.5.

E431. A method of synthesizing a therapeutic oligonucleotide formulated as a salt comprising one or more divalent cations, the method comprising heating an antisense strand and a sense strand in the presence of one or more divalent cations.

E432. The method of E431, wherein the heating comprises heating to at least 90° C.

E433. A method of preparing a therapeutic oligonucleotide formulated as a salt comprising one or more divalent cations, the method comprising incubating a hybridized siRNA duplex in the presence of one or more divalent cations without heat.

E434. The method of any one of E431-E433, wherein the therapeutic oligonucleotide is the therapeutic oligonucleotide of any one of E222-E400.

E435. A therapeutic oligonucleotide synthesized by the method of any one of E431-E434.

E436. A pharmaceutical composition comprising the therapeutic oligonucleotide of any one of E222-E430 and E435, and a pharmaceutically acceptable excipient, carrier, or diluent.

E437. The pharmaceutical composition of E436, wherein the salt is formulated as an aqueous solution.

E438. The pharmaceutical composition of E436, wherein the salt if formulated as a suspension.

E439. A kit comprising the branched siRNA molecule of any one E222-E430 and E435, or the pharmaceutical composition of any one of E436-E438, and a package insert, wherein the package insert instructs a user of the kit to perform the method of any one of E1-E221.

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure. Other embodiments are in the claims.

1. A method of delivering a short interfering RNA (siRNA) molecule to a subject, the method comprising administering the siRNA molecule to the central nervous system of the subject in the form of a salt comprising one or more divalent cations.

2. The method of claim 1, wherein the siRNA molecule comprises a plurality of cationic binding sites that are partially or fully saturated by the one or more divalent cations.

3. The method of claim 2, wherein the degree of saturation of the cationic binding sites by the one or more divalent cations is from 10% to 100%, optionally wherein the degree of saturation of the cationic binding sites by the one or more divalent cations is from 20% to 100%, from 30% to 100%, from 40% to 100%, from 50% to 100%, from 60% to 100%, from 70% to 100%, from 80% to 100%, or from 90% to 100%.

4. The method of any one of claims 1-3, wherein the cationic binding site is located within an internucleoside linkage, optionally wherein the internucleoside linkage is selected from a phosphodiester linkage and a phosphorothioate linkage.

5. The method of any one of claims 1-4, wherein the one or more divalent cations is characterized by an ionic radius of from 30 picometers to 150 picometers, optionally wherein the one or more divalent cations is characterized by an ionic radius of from 30 picometers to 140 picometers, from 40 picometers to 130 picometers, from 50 picometers to 120 picometers, from 60 picometers to 110 picometers, from 60 picometers to 100 picometers, or from 60 picometers to 90 picometers.

6. The method of any one of claims 1-5, wherein the one or more divalent cations comprise Ba²⁺, Be²⁺, Ca²⁺, Cu²⁺, Mg²⁺, Mn²⁺, Ni²⁺, or Zn²⁺, or a combination thereof.

7. The method of claim 6, wherein the one or more divalent cations comprise Ba²⁺.

8. The method of claim 6 or 7, wherein the one or more divalent cations comprise Be²⁺.

9. The method of any one of claims 6-8, wherein the one or more divalent cations comprise Ca²⁺.

10. The method of any one of claims 6-9, wherein the one or more divalent cations comprise Cu²⁺.

11. The method of any one of claims 6-10, wherein the one or more divalent cations comprise Mg²⁺.

12. The method of any one of claims 6-11, wherein the one or more divalent cations comprise Mn²⁺.

13. The method of any one of claims 6-12, wherein the one or more divalent cations comprise Ni²⁺.

14. The method of any one of claims 6-13, wherein the one or more divalent cations comprise Zn²⁺.

15. The method of any one of claims 6-14, wherein the one or more divalent cations comprise Ca²⁺ and Mg²⁺ optionally wherein the ratio of Ca²⁺ to Mg²⁺ is from 1:100 to 100:1.

16. The method of claim 15, wherein the Ca²⁺ and Mg²⁺ are present in a 1:1 molar ratio

17. The method of any one of claims 1-16, wherein the one or more divalent cations comprise a hard Lewis acid.

18. The method of any one of claims 1-17, wherein the one or more divalent cations displaces water from a cationic binding site of the siRNA molecule.

19. The method of any one of claims 1-18, wherein the siRNA molecule is non-branched.

20. The method of any one of claims 1-18, wherein the siRNA molecule is branched.

21. The method of claim 20, wherein the siRNA molecule is di-branched, tri-branched, or tetra-branched.

22. The method of claim 21, wherein the siRNA molecule is di-branched.

23. The method of any one of claims 1-22, wherein the siRNA molecule comprises an antisense strand and a sense strand having complementarity to the antisense strand.

24. The method of claim 23, wherein the antisense strand and sense strand comprises alternating 2′-O-methyl and 2′-fluoro ribonucleosides.

25. The method of claim 23 or 24, wherein the antisense strand has the following formula, in the 5′-to-3′ direction: 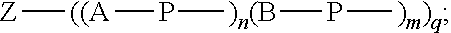
wherein Z is a 5′ phosphorus stabilizing moiety;
each A is, independently, a 2′-O-methyl (2′-O-Me) ribonucleoside;
each B is, independently, a 2′-fluoro (2′-F) ribonucleoside;
each P is, independently, an internucleoside linkage selected from a phosphodiester linkage and a phosphorothioate linkage;
n is an integer from 1 to 5;
m is an integer from 1 to 5; and
q is an integer between 1 and 30.

26. The method of claim 23 or 24, wherein the antisense strand comprises a structure represented by Formula I, wherein Formula I is, in the 5′-to-3′ direction: 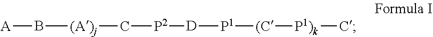
wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

27. The method of claim 26, wherein the antisense strand comprises a structure represented by Formula A1, wherein Formula A1 is, in the 5′-to-3′ direction: 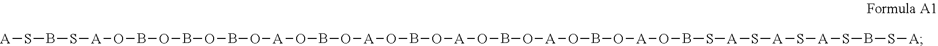
wherein A represents a 2′-O-Me ribonucleoside, B represents a 2′-F ribonucleoside, O represents a phosphodiester internucleoside linkage, and S represents a phosphorothioate internucleoside linkage.

28. The method of claim 23 or 24, wherein the antisense strand comprises a structure represented by Formula II, wherein Formula II is, in the 5′-to-3′ direction: 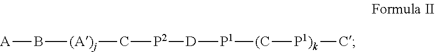
wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

29. The method of claim 28, wherein the antisense strand comprises a structure represented by Formula A2, wherein Formula A2 is, in the 5′-to-3′ direction: 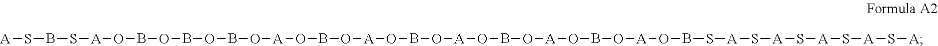
wherein A represents a 2′-O-Me ribonucleoside, B represents a 2′-F ribonucleoside, O represents a phosphodiester internucleoside linkage, and S represents a phosphorothioate internucleoside linkage.

30. The method of any one of claims 23-29, wherein the sense strand comprises a structure represented by Formula III, wherein Formula III is, in the 5′-to-3′ direction: 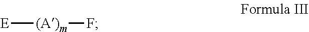
wherein E is represented by the formula (C—P¹)₂;
F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D;
A′, C, D, P¹, and P² are as defined in Formula II; and
m is an integer from 1 to 7.

31. The method of claim 30, wherein the sense strand comprises a structure represented by Formula S1, wherein Formula S1 is, in the 5′-to-3′ direction: 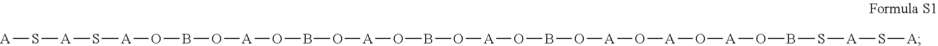
wherein A represents a 2′-O-Me ribonucleoside, B represents a 2′-F ribonucleoside, O represents a phosphodiester internucleoside linkage, and S represents a phosphorothioate internucleoside linkage.

32. The method of claim 30, wherein the sense strand comprises a structure represented by Formula S2, wherein Formula S2 is, in the 5′-to-3′ direction: 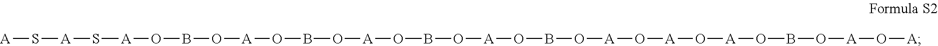
wherein A represents a 2′-O-Me ribonucleoside, B represents a 2′-F ribonucleoside, O represents a phosphodiester internucleoside linkage, and S represents a phosphorothioate internucleoside linkage.

33. The method of claim 30, wherein the sense strand comprises a structure represented by Formula S3, wherein Formula S3 is, in the 5′-to-3′ direction: 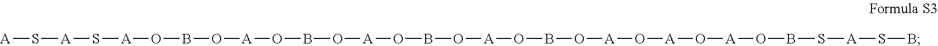
wherein A represents a 2′-O-Me ribonucleoside, B represents a 2′-F ribonucleoside, O represents a phosphodiester internucleoside linkage, and S represents a phosphorothioate internucleoside linkage.

34. The method of claim 30, wherein the sense strand comprises a structure represented by Formula S4, wherein Formula S4 is, in the 5′-to-3′ direction: 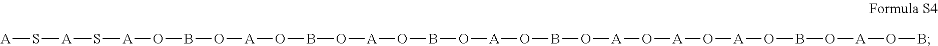
wherein A represents a 2′-O-Me ribonucleoside, B represents a 2′-F ribonucleoside, O represents a phosphodiester internucleoside linkage, and S represents a phosphorothioate internucleoside linkage.

35. The method of any one of claims 23, 24, and 30-34, wherein the antisense strand comprises a structure represented by Formula IV, wherein Formula IV is, in the 5′-to-3′ direction: 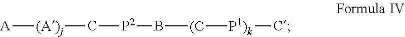
wherein A is represented by the formula C—P¹-D-P¹;
each A′ is represented by the formula C—P²-D-P²;
B is represented by the formula D-P¹—C—P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

36. The method of claim 35, wherein the antisense strand comprises a structure represented by Formula A3, wherein Formula A3 is, in the 5′-to-3′ direction: 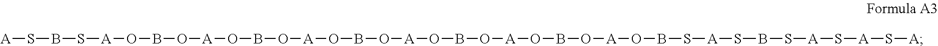
wherein A represents a 2′-O-Me ribonucleoside, B represents a 2′-F ribonucleoside, O represents a phosphodiester internucleoside linkage, and S represents a phosphorothioate internucleoside linkage.

37. The method of any one of claims 23-29, 35, and 36, wherein the sense strand comprises a structure represented by Formula V, wherein Formula V is, in the 5′-to-3′ direction: 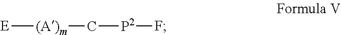
wherein E is represented by the formula (C—P¹)₂;
F is represented by the formula D-P¹—C—P¹—C, D-P²—C—P²—C, D-P¹—C—P¹-D, or D-P²—C—P²-D;
A′, C, D, P¹ and P² are as defined in Formula IV; and
m is an integer from 1 to 7.

38. The method of claim 37, wherein the sense strand comprises a structure represented by Formula S5, wherein Formula S5 is, in the 5′-to-3′ direction: 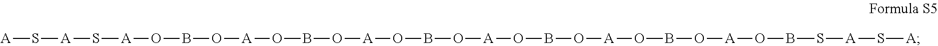
wherein A represents a 2′-O-Me ribonucleoside, B represents a 2′-F ribonucleoside, O represents a phosphodiester internucleoside linkage, and S represents a phosphorothioate internucleoside linkage.

39. The method of claim 37, wherein the sense strand comprises a structure represented by Formula S6, wherein Formula S6 is, in the 5′-to-3′ direction: 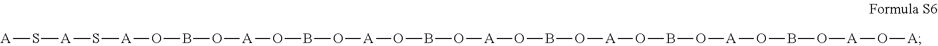
wherein A represents a 2′-O-Me ribonucleoside, B represents a 2′-F ribonucleoside, O represents a phosphodiester internucleoside linkage, and S represents a phosphorothioate internucleoside linkage.

40. The method of claim 37, wherein the sense strand comprises a structure represented by Formula S7, wherein Formula S7 is, in the 5′-to-3′ direction: 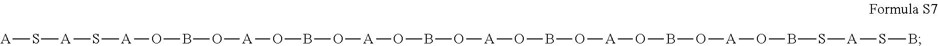
wherein A represents a 2′-O-Me ribonucleoside, B represents a 2′-F ribonucleoside, O represents a phosphodiester internucleoside linkage, and S represents a phosphorothioate internucleoside linkage.

41. The method of claim 37, wherein the sense strand comprises a structure represented by Formula S8, wherein Formula S8 is, in the 5′-to-3′ direction: 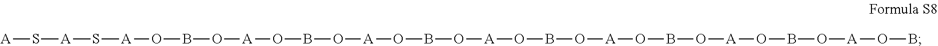
wherein A represents a 2′-O-Me ribonucleoside, B represents a 2′-F ribonucleoside, O represents a phosphodiester internucleoside linkage, and S represents a phosphorothioate internucleoside linkage.

42. The method of any one of claims 23, 24, 30-34, and 37-41, wherein the antisense strand comprises a structure represented by Formula VI, wherein Formula VI is, in the 5′-to-3′ direction: 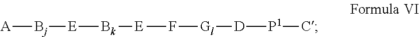
wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7;
k is an integer from 1 to 7; and
l is an integer from 1 to 7.

43. The method of claim 42, wherein the antisense strand comprises a structure represented by Formula A4, wherein Formula A4 is, in the 5′-to-3′ direction: 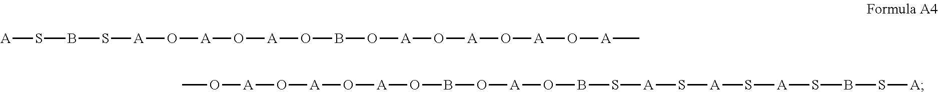
wherein A represents a 2′-O-Me ribonucleoside, B represents a 2′-F ribonucleoside, O represents a phosphodiester internucleoside linkage, and S represents a phosphorothioate internucleoside linkage.

44. The method of any one of claims 23-29, 35, 36, 42, and 43, wherein the sense strand comprises a structure represented by Formula VII, wherein Formula VII is, in the 5′-to-3′ direction: 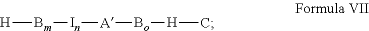
wherein A′ is represented by the formula C—P²-D-P²;
each H is represented by the formula (C—P¹)₂;
each I is represented by the formula (D-P²);
B, C, D, P¹ and P² are as defined in Formula VI;
m is an integer from 1 to 7;
n is an integer from 1 to 7; and
o is an integer from 1 to 7.

45. The method of claim 44, wherein the sense strand comprises a structure represented by Formula s9, wherein Formula S9 is, in the 5′-to-3′ direction: 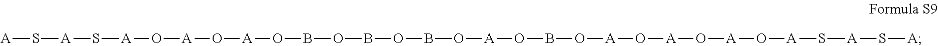
wherein A represents a 2′-O-Me ribonucleoside, B represents a 2′-F ribonucleoside, O represents a phosphodiester internucleoside linkage, and S represents a phosphorothioate internucleoside linkage.

46. The method of any one of claims 23-45, wherein the antisense strand further comprises a 5′-phosphorus stabilizing moiety at the 5′ end of the antisense strand.

47. The method of any one of claims 23-46, wherein the sense strand further comprises a 5′-phosphorus stabilizing moiety at the 5′ end of the sense strand.

48. The method of any one of claims 25, 46, and 47, wherein the 5′-phosphorus stabilizing moiety is represented by any one of Formulas IX-XVI: 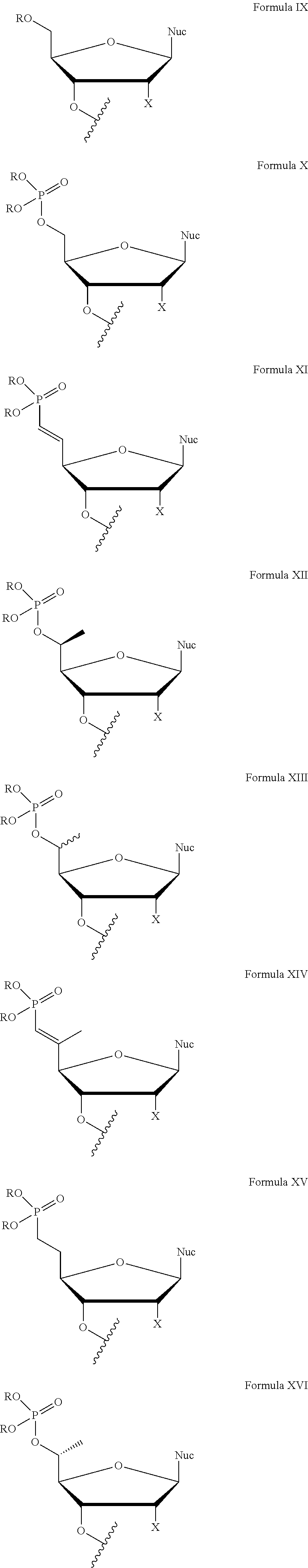
wherein Nuc represents a nucleobase selected from the group consisting of adenine, uracil, guanine, thymine, and cytosine, and R represents optionally substituted alkyl, optionally substituted alkenyl, optionally substituted alkynyl, phenyl, benzyl, hydroxy, or hydrogen.

49. The method of claim 48, wherein the 5′-phosphorus stabilizing moiety is (E)-vinylphosphonate represented in Formula XVI.

50. The method of any one of claims 24-49, wherein at least 50% of the ribonucleosides are 2′-O-Me ribonucleosides, optionally wherein at least 60%, 70%, 80%, 90%, or more of the ribonucleosides are 2′-O-Me ribonucleosides.

51. The method of any one of claims 23-50, wherein the length of the antisense strand is between 10 and 30 nucleotides.

52. The method of any one of claims 23-51, wherein the length of the sense strand is between 12 and 30 nucleotides.

53. The method of any one of claims 1-52, wherein the siRNA molecule is administered in the form of an aqueous solution or in the form of a suspension.

54. The method of claim 1-53, wherein the siRNA molecule is administered directly to the cerebral spinal fluid of the subject, directly to the spinal cord of the subject, and/or directly to the brain parenchyma of the subject, optionally wherein (i) the siRNA molecule being administered to the brain is specifically administered to the cortex, cerebellum, basal ganglia, or other brain structure and/or (ii) the siRNA molecule being administered to the basal ganglia is specifically administered to the caudate, putamen, thalamus, globus pallidus, or substantia nigra.

55. The method of any one of claims 1-54, wherein the siRNA molecule is administered intrathecally, intracerebroventricularly, intrastriatally, or by intra-cisterna magna injection via catheterization.

56. The method of any one of claims 1-55, wherein the subject is diagnosed as having Huntington's disease, Parkinson's disease, Alzheimer's disease, amyotrophic lateral sclerosis (ALS), dementia with Lewy bodies (DLB), pure autonomic failure, Lewy body dysphagia, incidental Lewy body disease (ILBD), inherited Lewy body disease, olivopontocerebellar atrophy (OPCA), striatonigral degeneration, Shy-Drager syndrome, epilepsy or an epilepsy disorder, a prion disease, or a pain disorder.

57. The method of any one of claims 23-56, wherein the antisense strand has complementarity sufficient to hybridize a portion of an mRNA transcript corresponding to a gene selected from the group consisting of ABCA7, ABI3, ADAM10, APOC1, APOE, AXL, BIN1, C1QA, C3, C90RF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, HTT, IFIT1, IFIT3, IFITM3, IFNAR1, IFNAR2, IGF1, IL10RA, IL1A, IL1B, IL1RAP, INPP5D, ITGAM, ITGAX, KCNT1, LILRB4, LPL, MAPT, MEF2C, MMP12, MS4A4A, MS4A6A, MSH3, NLRP3, NME8, NOS2, PICALM, PILRA, PLCG2, PRNP, PTK2B, SCIMP, SCN9A, SLC24A4, SNCA, SORL1, SPI1, SPP1, SPPL2A, TBK1, TNF, TREM2, TREML2, TYROBP, and ZCWPW1.

58. The method of claim 57, wherein the gene is selected from the group consisting of HTT, MAPT, SNCA, C90RF72, APOE, SCN9A, KCNT1, PRNP, and MSH3.

59. The method of any one of claims 1-58, wherein the subject is a human.

60. The method of any one of claims 1-59, wherein the therapeutic oligonucleotide comprises one or more atoms having a negative charge and the divalent cation comprises two positive charges, and wherein the ratio of negative charge to positive charge is from 0.75 to 7.5, optionally wherein the ratio of negative charge to positive charge is from 1.0 to 2.0.

61. The method of claim 60, wherein
i. the ratio of negative charge to positive charge is from 0.75 to 6.5, optionally wherein the ratio of negative charge to positive charge is from 0.75 to 5.5, from 0.75 to 4.5, from 0.75 to 3.5, from 0.75 to 2.5, from 0.75 to 1.5, or from 0.75 to 1, or
ii. the ratio of negative charge to positive charge is from 1 to 7.5, from 1.5 to 7.5, from 2.5 to 7.5, from 3.5 to 7.5, from 4.5 to 7.5, from 5.5 to 7.5, or from 6.5 to 7.5.

62. The method of any one of claims 1-61, wherein the molar ratio of siRNA molecule to the one or more divalent cations is from 1:10 to 1:100.

63. The method of claim 62, wherein the molar ratio of siRNA molecule to the one or more divalent cations is from 1:10 to 1:50, optionally wherein the molar ratio of siRNA molecule to the one or more divalent cations is from 1:18 to 1:38, optionally wherein the molar ratio of siRNA molecule to the one or more divalent cations is from 1:20 to 1:25, optionally wherein the molar ratio of siRNA molecule to the one or more divalent cations is about 1:20, optionally wherein the molar ratio of siRNA molecule to the one or more divalent cations is about 1:25.

64. The method of any one of claims 1-63, wherein the concentration of the one or more divalent cations is from 10 mM to 150 mM.

65. The method of claim 64, wherein the concentration of the one or more divalent cations is from 20 mM to 150 mM, optionally wherein the concentration of the one or more divalent cations is from 20 mM to 100 mM, from 25 mM to 150 mM, from 25 mM to 100 mM, from 30 mM to 90 mM, from 35 mM to 85 mM, from 35 mM to 75 mM, from 40 mM to 70 mM, from 40 mM to 65 mM, from 40 mM to 60 mM, or from 40 mM to 50 mM.

66. An siRNA molecule formulated as a salt comprising one or more divalent cations.

67. The siRNA molecule of claim 66, wherein the siRNA molecule comprises a plurality of cationic binding sites that are partially or fully saturated by the one or more divalent cations.

68. The siRNA molecule of claim 67, wherein the degree of saturation of the cationic binding sites by the one or more divalent cations is from 10% to 100%, optionally wherein the degree of saturation of the cationic binding sites by the one or more divalent cations is from 20% to 100%, from 30% to 100%, from 40% to 100%, from 50% to 100%, from 60% to 100%, from 70% to 100%, from 80% to 100%, or from 90% to 100%.

69. The siRNA molecule of any one of claims 66-68, wherein the cationic binding site is located within an internucleoside linkage, optionally wherein the internucleoside linkage is selected from a phosphodiester linkage and a phosphorothioate linkage.

70. The siRNA molecule of any one of claims 66-69, wherein the one or more divalent cations is characterized by an ionic radius of from 30 picometers to 150 picometers, optionally wherein the one or more divalent cations is characterized by an ionic radius of from 30 picometers to 140 picometers, from 40 picometers to 130 picometers, from 50 picometers to 120 picometers, from 60 picometers to 110 picometers, from 60 picometers to 100 picometers, or from 60 picometers to 90 picometers.

71. The siRNA molecule of any one of claims 66-70, wherein the one or more divalent cations comprise Ba²⁺, Be²⁺, Ca²⁺, Cu²⁺, Mg²⁺, Mn²⁺, Ni²⁺, or Zn²⁺, or a combination thereof.

72. The siRNA molecule of any one of claims 66-71, wherein the one or more divalent cations comprise a hard Lewis acid.

73. The siRNA molecule of any one of claims 66-72, wherein the one or more divalent cations displaces water from a cationic binding site of the siRNA molecule.

74. The siRNA molecule of any one of claims 66-73, wherein the siRNA molecule is non-branched.

75. The siRNA molecule of any one of claims 66-73, wherein the siRNA molecule is branched.

76. The siRNA molecule of claim 75, wherein the siRNA molecule is di-branched, tri-branched, or tetra-branched, optionally wherein the siRNA molecule is di-branched.

77. The siRNA molecule of any one of claims 66-76, wherein the siRNA molecule comprises an antisense strand and a sense strand having complementarity to the antisense strand, optionally wherein the antisense strand and sense strand comprise alternating 2′-O-methyl and 2′-fluoro ribonucleosides.

78. The siRNA molecule of claim 77, wherein the antisense strand has the following formula, in the 5′-to-3′ direction: 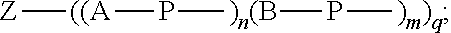
wherein Z is a 5′ phosphorus stabilizing moiety;
each A is, independently, a 2′-O-methyl (2′-O-Me) ribonucleoside;
each B is, independently, a 2′-fluoro (2′-F) ribonucleoside;
each P is, independently, an internucleoside linkage selected from a phosphodiester linkage and a phosphorothioate linkage;
n is an integer from 1 to 5;
m is an integer from 1 to 5; and
q is an integer between 1 and 30.

79. The siRNA molecule of claim 77, wherein the antisense strand comprises a structure represented by Formula I, wherein Formula I is, in the 5′-to-3′ direction: 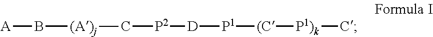
wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

80. The siRNA molecule of claim 79, wherein the antisense strand comprises a structure represented by Formula A1, wherein Formula A1 is, in the 5′-to-3′ direction: 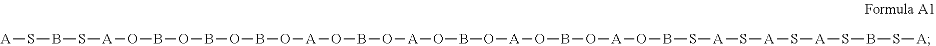
wherein A represents a 2′-O-Me ribonucleoside, B represents a 2′-F ribonucleoside, O represents a phosphodiester internucleoside linkage, and S represents a phosphorothioate internucleoside linkage.

81. The siRNA molecule of claim 77, wherein the antisense strand comprises a structure represented by Formula II, wherein Formula II is, in the 5′-to-3′ direction: 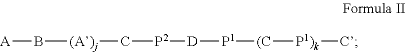
wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

82. The siRNA molecule of claim 81, wherein the antisense strand comprises a structure represented by Formula A2, wherein Formula A2 is, in the 5′-to-3′ direction: 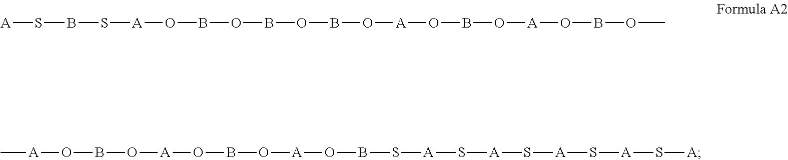
wherein A represents a 2′-O-Me ribonucleoside, B represents a 2′-F ribonucleoside, O represents a phosphodiester internucleoside linkage, and S represents a phosphorothioate internucleoside linkage.

83. The siRNA molecule of any one of claims 77-82, wherein the sense strand comprises a structure represented by Formula III, wherein Formula III is, in the 5′-to-3′ direction: 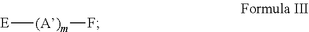
wherein E is represented by the formula (C—P¹)₂;
F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D;
A′, C, D, P¹, and P² are as defined in Formula II; and
m is an integer from 1 to 7.

84. The siRNA molecule of claim 83, wherein the sense strand comprises a structure represented by Formula S1, wherein Formula S1 is, in the 5′-to-3′ direction: 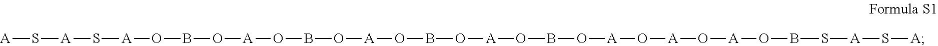
wherein A represents a 2′-O-Me ribonucleoside, B represents a 2′-F ribonucleoside, O represents a phosphodiester internucleoside linkage, and S represents a phosphorothioate internucleoside linkage.

85. The siRNA molecule of claim 83, wherein the sense strand comprises a structure represented by Formula S2, wherein Formula S2 is, in the 5′-to-3′ direction: 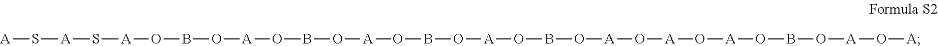
wherein A represents a 2′-O-Me ribonucleoside, B represents a 2′-F ribonucleoside, O represents a phosphodiester internucleoside linkage, and S represents a phosphorothioate internucleoside linkage.

86. The siRNA molecule of claim 83, wherein the sense strand comprises a structure represented by Formula S3, wherein Formula S3 is, in the 5′-to-3′ direction: 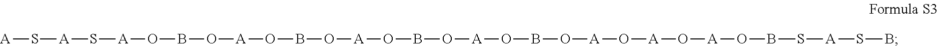
wherein A represents a 2′-O-Me ribonucleoside, B represents a 2′-F ribonucleoside, O represents a phosphodiester internucleoside linkage, and S represents a phosphorothioate internucleoside linkage.

87. The siRNA molecule of claim 83, wherein the sense strand comprises a structure represented by Formula S4, wherein Formula S4 is, in the 5′-to-3′ direction: 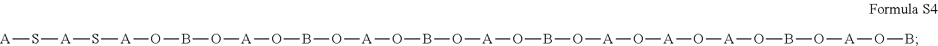
wherein A represents a 2′-O-Me ribonucleoside, B represents a 2′-F ribonucleoside, O represents a phosphodiester internucleoside linkage, and S represents a phosphorothioate internucleoside linkage.

88. The siRNA molecule of any one of claims 77 and 83-87, wherein the antisense strand comprises a structure represented by Formula IV, wherein Formula IV is, in the 5′-to-3′ direction: 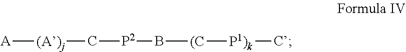
wherein A is represented by the formula C—P¹-D-P¹;
each A′ is represented by the formula C—P²-D-P²;
B is represented by the formula D-P¹—C—P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

89. The siRNA molecule of claim 88, wherein the antisense strand comprises a structure represented by Formula A3, wherein Formula A3 is, in the 5′-to-3′ direction: 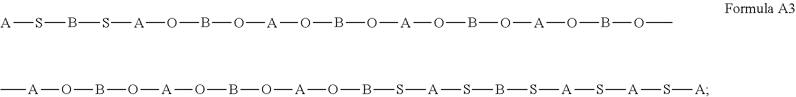
wherein A represents a 2′-O-Me ribonucleoside, B represents a 2′-F ribonucleoside, O represents a phosphodiester internucleoside linkage, and S represents a phosphorothioate internucleoside linkage.

90. The siRNA molecule of any one of claims 77-82, 88, and 89, wherein the sense strand comprises a structure represented by Formula V, wherein Formula V is, in the 5′-to-3′ direction: 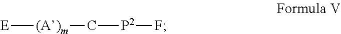
wherein E is represented by the formula (C—P¹)₂;
F is represented by the formula D-P¹—C—P¹—C, D-P²—C—P²—C, D-P¹—C—P¹-D, or D-P²—C—P²-D;
A′, C, D, P¹ and P² are as defined in Formula IV; and
m is an integer from 1 to 7.

91. The siRNA molecule of claim 90, wherein the sense strand comprises a structure represented by Formula S5, wherein Formula S5 is, in the 5′-to-3′ direction: 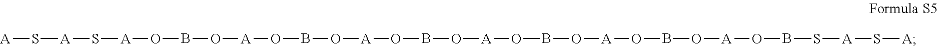
wherein A represents a 2′-O-Me ribonucleoside, B represents a 2′-F ribonucleoside, O represents a phosphodiester internucleoside linkage, and S represents a phosphorothioate internucleoside linkage.

92. The siRNA molecule of claim 90, wherein the sense strand comprises a structure represented by Formula S6, wherein Formula S6 is, in the 5′-to-3′ direction: 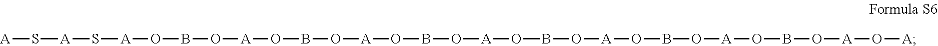
wherein A represents a 2′-O-Me ribonucleoside, B represents a 2′-F ribonucleoside, O represents a phosphodiester internucleoside linkage, and S represents a phosphorothioate internucleoside linkage.

93. The siRNA molecule of claim 90, wherein the sense strand comprises a structure represented by Formula S7, wherein Formula S7 is, in the 5′-to-3′ direction: 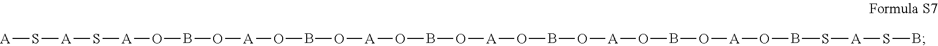
wherein A represents a 2′-O-Me ribonucleoside, B represents a 2′-F ribonucleoside, O represents a phosphodiester internucleoside linkage, and S represents a phosphorothioate internucleoside linkage.

94. The siRNA molecule of claim 90, wherein the sense strand comprises a structure represented by Formula S8, wherein Formula S8 is, in the 5′-to-3′ direction: 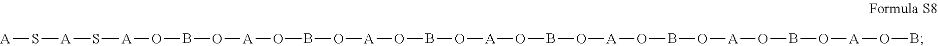
wherein A represents a 2′-O-Me ribonucleoside, B represents a 2′-F ribonucleoside, O represents a phosphodiester internucleoside linkage, and S represents a phosphorothioate internucleoside linkage.

95. The siRNA molecule of any one of claims 77, 83-87, and 90-94, wherein the antisense strand comprises a structure represented by Formula VI, wherein Formula VI is, in the 5′-to-3′ direction: 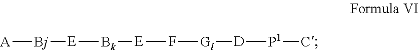
wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7;
k is an integer from 1 to 7; and
l is an integer from 1 to 7.

96. The siRNA molecule of claim 95, wherein the antisense strand comprises a structure represented by Formula A4, wherein Formula A4 is, in the 5′-to-3′ direction: 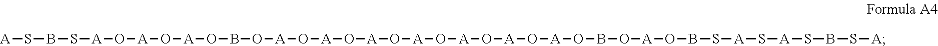
wherein A represents a 2′-O-Me ribonucleoside, B represents a 2′-F ribonucleoside, O represents a phosphodiester internucleoside linkage, and S represents a phosphorothioate internucleoside linkage.

97. The siRNA molecule of any one of claims 77-82, 88, 89, 95, and 96, wherein the sense strand comprises a structure represented by Formula VII, wherein Formula VII is, in the 5′-to-3′ direction: 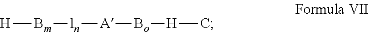
wherein A′ is represented by the formula C—P²-D-P²;
each H is represented by the formula (C—P¹)₂;
each I is represented by the formula (D-P²);
B, C, D, P¹ and P² are as defined in Formula VI;
m is an integer from 1 to 7;
n is an integer from 1 to 7; and
o is an integer from 1 to 7.

98. The siRNA molecule of claim 97, wherein the sense strand comprises a structure represented by Formula s9, wherein Formula S9 is, in the 5′-to-3′ direction: 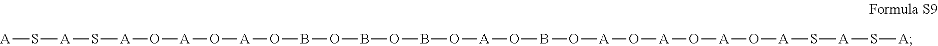
wherein A represents a 2′-O-Me ribonucleoside, B represents a 2′-F ribonucleoside, O represents a phosphodiester internucleoside linkage, and S represents a phosphorothioate internucleoside linkage.

99. The siRNA molecule of any one of claims 77-98, wherein the antisense strand further comprises a 5′-phosphorus stabilizing moiety at the 5′ end of the antisense strand.

100. The siRNA molecule of any one of claims 77-99, wherein the sense strand further comprises a 5′-phosphorus stabilizing moiety at the 5′ end of the sense strand.

101. The siRNA molecule of claim 99 or 100, wherein the 5′-phosphorus stabilizing moiety is represented by any one of Formulas IX-XVI: 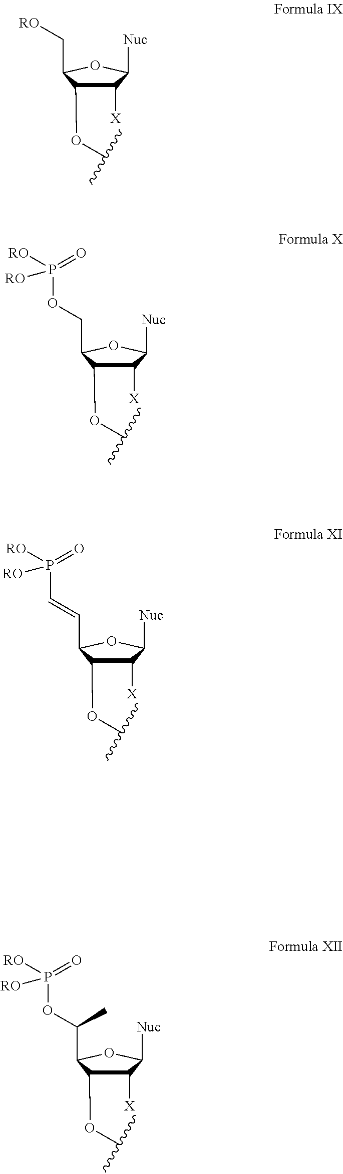 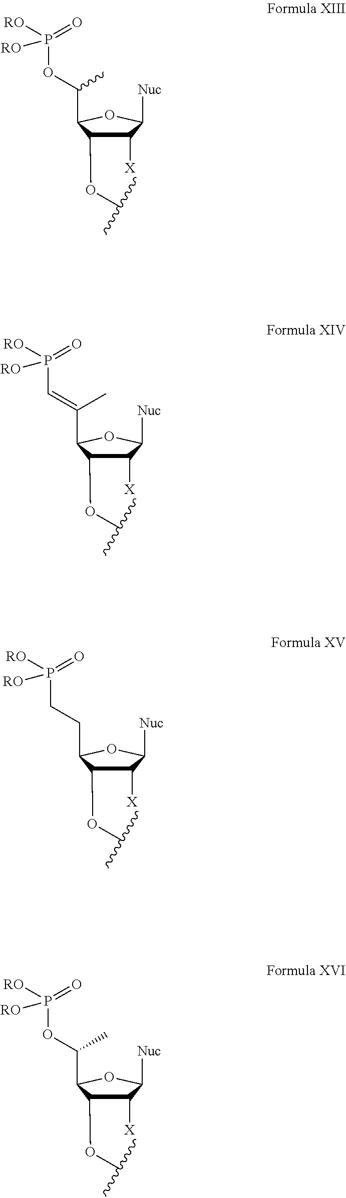
wherein Nuc represents a nucleobase selected from the group consisting of adenine, uracil, guanine, thymine, and cytosine, and R represents optionally substituted alkyl, optionally substituted alkenyl, optionally substituted alkynyl, phenyl, benzyl, hydroxy, or hydrogen.

102. The siRNA molecule of any one of claims 66-101, wherein the antisense strand has complementarity sufficient to hybridize a portion of an mRNA transcript corresponding to a gene selected from the group consisting ofABCA7, ABI3, ADAM10, APOC1, APOE, AXL, BIN1, C1QA, C3, C90RF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, HTT, IFIT1, IFIT3, IFITM3, IFNAR1, IFNAR2, IGF1, IL10RA, IL1A, IL1B, IL1RAP, INPP5D, ITGAM, ITGAX, KCNT1, LILRB4, LPL, MAPT, MEF2C, MMP12, MS4A4A, MS4A6A, MSH3, NLRP3, NME8, NOS2, PICALM, PILRA, PLCG2, PRNP, PTK2B, SCIMP, SCN9A, SLC24A4, SNCA, SORL1, SPI1, SPP1, SPPL2A, TBK1, TNF, TREM2, TREML2, TYROBP, and ZCWPW1.

103. The siRNA molecule of claim 102, wherein the gene is selected from the group consisting of HTT, MAPT, SNCA, C90RF72, APOE, SCN9A, KCNT1, PRNP, and MSH3.

104. The siRNA molecule of any one of claims 66-103, wherein the therapeutic oligonucleotide comprises one or more atoms having a negative charge and the divalent cation comprises two positive charges, and wherein the ratio of negative charge to positive charge is from 0.75 to 7.5, optionally wherein the ratio of negative charge to positive charge is from 1.0 to 2.0.

105. The siRNA molecule of claim 104, wherein:
i. the ratio of negative charge to positive charge is from 0.75 to 6.5, optionally wherein the ratio of negative charge to positive charge is from 0.75 to 5.5, from 0.75 to 4.5, from 0.75 to 3.5, from 0.75 to 2.5, from 0.75 to 1.5, or from 0.75 to 1; or
ii. the ratio of negative charge to positive charge is from 1 to 7.5, from 1.5 to 7.5, from 2.5 to 7.5, from 3.5 to 7.5, from 4.5 to 7.5, from 5.5 to 7.5, or from 6.5 to 7.5.

106. The siRNA molecule of any one of claims 66-105, wherein the molar ratio of siRNA molecule to the one or more divalent cations is from 1:10 to 1:100.

107. The siRNA molecule of claim 106, wherein the molar ratio of siRNA molecule to the one or more divalent cations is from 1:10 to 1:50, optionally wherein the molar ratio of siRNA molecule to the one or more divalent cations is from 1:18 to 1:38, optionally wherein the molar ratio of siRNA molecule to the one or more divalent cations is from 1:20 to 1:25, optionally wherein the molar ratio of siRNA molecule to the one or more divalent cations is about 1:20, optionally wherein the molar ratio of siRNA molecule to the one or more divalent cations is about 1:25.

108. The siRNA molecule of any one of claims 66-107, wherein the concentration of the one or more divalent cations is from 10 mM to 150 mM.

109. The siRNA molecule of claim 108, wherein the concentration of the one or more divalent cations is from 20 mM to 150 mM, optionally wherein the concentration of the one or more divalent cations is from 20 mM to 100 mM, from 25 mM to 150 mM, from 25 mM to 100 mM, from 30 mM to 90 mM, from 35 mM to 85 mM, from 35 mM to 75 mM, from 40 mM to 70 mM, from 40 mM to 65 mM, from 40 mM to 60 mM, or from 40 mM to 50 mM.

110. A therapeutic oligonucleotide formulated as a salt comprising one or more divalent cations, wherein:
i) the one or more divalent cations comprise Mg²⁺, Ba²⁺, Be²⁺, Cu²⁺, Mn²⁺, Ni²⁺, or Zn²⁺, or a combination thereof, optionally wherein the one or more divalent cations comprise Mg²⁺; and/or
ii) the therapeutic oligonucleotide comprises one or more atoms having a negative charge and the divalent cation comprises two positive charges, and wherein the ratio of negative charge to positive charge is from 0.75 to 7.5, optionally wherein the ratio of negative charge to positive charge is from 1.0 to 2.0; and/or
iii) the molar ratio of therapeutic oligonucleotide to the one or more divalent cations is from 1:10 to 1:100; and/or
iv) the concentration of the one or more divalent cations is from 10 mM to 150 mM.

111. The therapeutic oligonucleotide of claim 110, wherein the therapeutic oligonucleotide comprises a plurality of cationic binding sites that are partially or fully saturated by the one or more divalent cations.

112. The therapeutic oligonucleotide of claim 111, wherein the degree of saturation of the cationic binding sites by the one or more divalent cations is from 10% to 100%, optionally wherein the degree of saturation of the cationic binding sites by the one or more divalent cations is from 20% to 100%, from 30% to 100%, from 40% to 100%, from 50% to 100%, from 60% to 100%, from 70% to 100%, from 80% to 100%, or from 90% to 100%.

113. The therapeutic oligonucleotide of any one of claims 110-112, wherein the cationic binding site is located within an internucleoside linkage, optionally wherein the internucleoside linkage is selected from a phosphodiester linkage and a phosphorothioate linkage.

114. The therapeutic oligonucleotide of any one of claims 110-113, wherein the one or more divalent cations is characterized by an ionic radius of from 30 picometers to 150 picometers, optionally wherein the one or more divalent cations is characterized by an ionic radius of from 30 picometers to 140 picometers, from 40 picometers to 130 picometers, from 50 picometers to 120 picometers, from 60 picometers to 110 picometers, from 60 picometers to 100 picometers, or from 60 picometers to 90 picometers.

115. The therapeutic oligonucleotide of any one of claims 110-114, wherein the one or more divalent cations comprise Mg²⁺.

116. The therapeutic oligonucleotide of any one of claims 110-115, wherein the one or more divalent cations comprise a hard Lewis acid.

117. The therapeutic oligonucleotide of any one of claims 110-116, wherein the one or more divalent cations displaces water from a cationic binding site of the siRNA molecule.

118. The therapeutic oligonucleotide of any one of claims 110-117, wherein the therapeutic oligonucleotide comprises alternating 2′-O-methyl and 2′-fluoro ribonucleosides.

119. The therapeutic oligonucleotide of any one of claims 110-118, wherein the therapeutic oligonucleotide has the following formula, in the 5′-to-3′ direction: 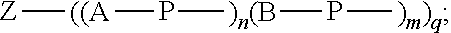
wherein Z is a 5′ phosphorus stabilizing moiety;
each A is, independently, a 2′-O-methyl (2′-O-Me) ribonucleoside;
each B is, independently, a 2′-fluoro (2′-F) ribonucleoside;
each P is, independently, an internucleoside linkage selected from a phosphodiester linkage and a phosphorothioate linkage;
n is an integer from 1 to 5;
m is an integer from 1 to 5; and
q is an integer between 1 and 30.

120. The therapeutic oligonucleotide of any one of claims 110-118, wherein the therapeutic oligonucleotide comprises a structure represented by Formula I, wherein Formula I is, in the 5′-to-3′ direction: 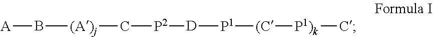
wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

121. The therapeutic oligonucleotide of claim 120, wherein the therapeutic oligonucleotide comprises a structure represented by Formula A1, wherein Formula A1 is, in the 5′-to-3′ direction: 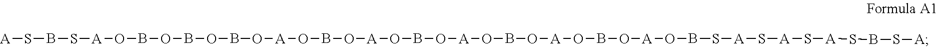
wherein A represents a 2′-O-Me ribonucleoside, B represents a 2′-F ribonucleoside, O represents a phosphodiester internucleoside linkage, and S represents a phosphorothioate internucleoside linkage.

122. The therapeutic oligonucleotide of any one of claims 110-118, wherein the therapeutic oligonucleotide comprises a structure represented by Formula II, wherein Formula II is, in the 5′-to-3′ direction: 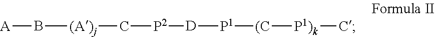
wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

123. The therapeutic oligonucleotide of claim 122, wherein the therapeutic oligonucleotide comprises a structure represented by Formula A2, wherein Formula A2 is, in the 5′-to-3′ direction: 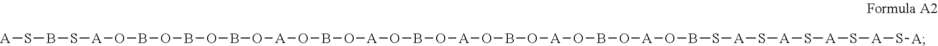
wherein A represents a 2′-O-Me ribonucleoside, B represents a 2′-F ribonucleoside, O represents a phosphodiester internucleoside linkage, and S represents a phosphorothioate internucleoside linkage.

124. The therapeutic oligonucleotide of any one of claims 110-118, wherein the therapeutic oligonucleotide comprises a structure represented by Formula IV, wherein Formula IV is, in the 5′-to-3′ direction: 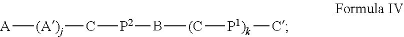
wherein A is represented by the formula C—P¹-D-P¹;
each A′ is represented by the formula C—P²-D-P²;
B is represented by the formula D-P¹—C—P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

125. The therapeutic oligonucleotide of claim 124, wherein the therapeutic oligonucleotide comprises a structure represented by Formula A3, wherein Formula A3 is, in the 5′-to-3′ direction: 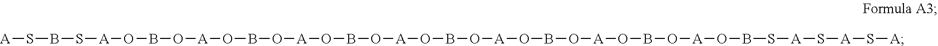
wherein A represents a 2′-O-Me ribonucleoside, B represents a 2′-F ribonucleoside, O represents a phosphodiester internucleoside linkage, and S represents a phosphorothioate internucleoside linkage.

126. The therapeutic oligonucleotide of any one of claims 110-118, wherein the therapeutic oligonucleotide comprises a structure represented by Formula VI, wherein Formula VI is, in the 5′-to-3′ direction: 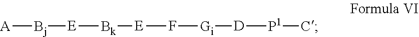
wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7;
k is an integer from 1 to 7; and
l is an integer from 1 to 7.

127. The therapeutic oligonucleotide of claim 126, wherein the therapeutic oligonucleotide comprises a structure represented by Formula A4, wherein Formula A4 is, in the 5′-to-3′ direction: 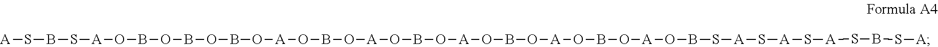
wherein A represents a 2′-O-Me ribonucleoside, B represents a 2′-F ribonucleoside, 0 represents a phosphodiester internucleoside linkage, and S represents a phosphorothioate internucleoside linkage.

128. The therapeutic oligonucleotide of any one of claims 110-127, wherein the therapeutic oligonucleotide further comprises a 5′-phosphorus stabilizing moiety at the 5′ end of the therapeutic oligonucleotide.

129. The therapeutic oligonucleotide of claim 128, wherein the 5′-phosphorus stabilizing moiety is represented by any one of Formulas IX-XVI: 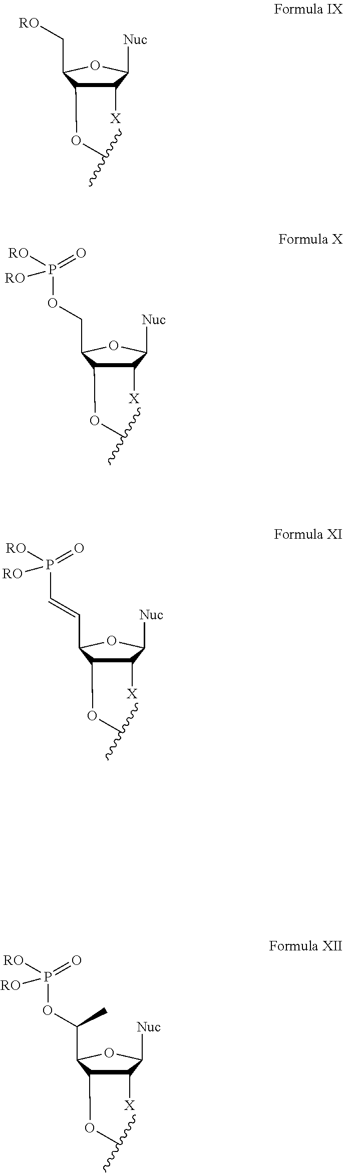 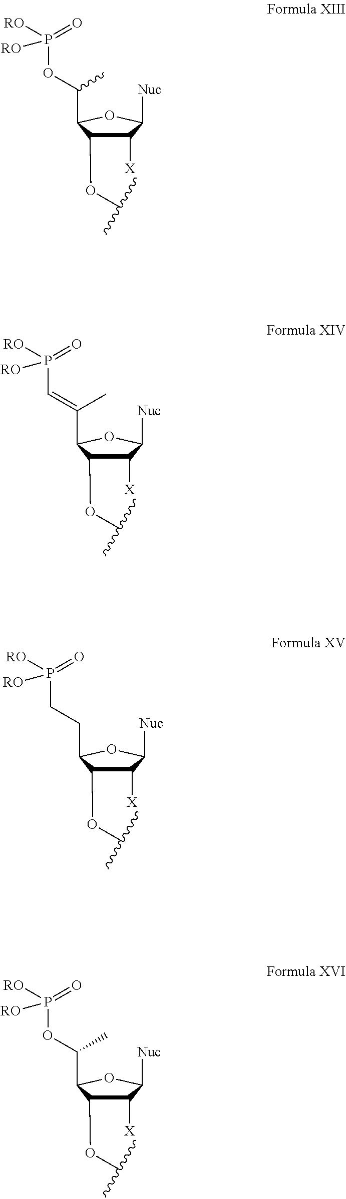
wherein Nuc represents a nucleobase selected from the group consisting of adenine, uracil, guanine, thymine, and cytosine, and R represents optionally substituted alkyl, optionally substituted alkenyl, optionally substituted alkynyl, phenyl, benzyl, hydroxy, or hydrogen.

130. The therapeutic oligonucleotide of any one of claims 110-129, wherein the therapeutic oligonucleotide has complementarity sufficient to hybridize a portion of an mRNA transcript corresponding to a gene selected from the group consisting of ABCA7, ABI3, ADAM10, APOC1, APOE, AXL, BIN1, C1QA, C3, C90RF72, CASS4, CCL5, CD2AP, CD33, CD68, CLPTM1, CLU, CR1, CSF1, CST7, CTSB, CTSD, CTSL, CXCL10, CXCL13, DSG2, ECHDC3, EPHA1, FABP5, FERMT2, FTH1, GNAS, GRN, HBEGF, HLA-DRB1, HLA-DRB5, HTT, IFIT1, IFIT3, IFITM3, IFNAR1, IFNAR2, IGF1, IL10RA, IL1A, IL1B, IL1 RAP, INPP5D, ITGAM, ITGAX, KCNT1, LILRB4, LPL, MAPT, MEF2C, MMP12, MS4A4A, MS4A6A, MSH3, NLRP3, NME8, NOS2, PICALM, PILRA, PLCG2, PRNP, PTK2B, SCIMP, SCN9A, SLC24A4, SNCA, SORL1, SPI1, SPP1, SPPL2A, TBK1, TNF, TREM2, TREML2, TYROBP, and ZCWPW1.

131. The therapeutic oligonucleotide of claim 130, wherein the gene is selected from the group consisting of HTT, MAPT, SNCA, C90RF72, APOE, SCN9A, KCNT1, PRNP, and MSH3.

132. The therapeutic oligonucleotide of any one of claims 110-131, wherein the therapeutic oligonucleotide comprises one or more atoms having a negative charge and the divalent cation comprises two positive charges, and wherein the ratio of negative charge to positive charge is from 0.75 to 7.5, optionally wherein the ratio of negative charge to positive charge is from 1.0 to 2.0.

133. The therapeutic oligonucleotide of claim 132, wherein:
i. the ratio of negative charge to positive charge is from 0.75 to 6.5, optionally wherein the ratio of negative charge to positive charge is from 0.75 to 5.5, from 0.75 to 4.5, from 0.75 to 3.5, from 0.75 to 2.5, from 0.75 to 1.5, or from 0.75 to 1, or
ii. the ratio of negative charge to positive charge is from 1 to 7.5, from 1.5 to 7.5, from 2.5 to 7.5, from 3.5 to 7.5, from 4.5 to 7.5, from 5.5 to 7.5, or from 6.5 to 7.5.

134. The therapeutic oligonucleotide of any one of claims 110-133, wherein the molar ratio of therapeutic oligonucleotide to the one or more divalent cations is from 1:10 to 1:100.

135. The therapeutic oligonucleotide of claim 134, wherein the molar ratio of therapeutic oligonucleotide to the one or more divalent cations is from 1:10 to 1:50, optionally wherein the molar ratio of therapeutic oligonucleotide to the one or more divalent cations is from 1:18 to 1:38, optionally wherein the molar ratio of therapeutic oligonucleotide to the one or more divalent cations is from 1:20 to 1:25, optionally wherein the molar ratio of therapeutic oligonucleotide to the one or more divalent cations is about 1:20, optionally wherein the molar ratio of therapeutic oligonucleotide to the one or more divalent cations is about 1:25.

136. The therapeutic oligonucleotide of any one of claims 110-135, wherein the concentration of the one or more divalent cations is from 10 mM to 150 mM.

137. The therapeutic oligonucleotide of claim 136, wherein the concentration of the one or more divalent cations is from 20 mM to 150 mM, optionally wherein the concentration of the one or more divalent cations is from 20 mM to 100 mM, from 25 mM to 150 mM, from 25 mM to 100 mM, from 30 mM to 90 mM, from 35 mM to 85 mM, from 35 mM to 75 mM, from 40 mM to 70 mM, from 40 mM to 65 mM, from 40 mM to 60 mM, or from 40 mM to 50 mM.

138. The therapeutic oligonucleotide of any one of claims 110-137, wherein the therapeutic oligonucleotide is an antisense oligonucleotide (ASO).

139. The therapeutic oligonucleotide of any one of claims 110-137, wherein the therapeutic oligonucleotide is an interfering RNA molecule, optionally wherein the interfering RNA molecule is an siRNA, an miRNA, or an shRNA.

140. The therapeutic oligonucleotide of claim 139, wherein the therapeutic oligonucleotide is an siRNA.

141. A kit comprising the siRNA molecule of any one of claims 66-109 and a package insert, optionally wherein the package insert instructs a user of the kit to administer the siRNA molecule to the central nervous system of a human subject.

142. A kit comprising the therapeutic oligonucleotide of any one of claims 110-140 and a package insert, optionally wherein the package insert instructs a user of the kit to administer the ASO to the central nervous system of a human subject.
